(19) United States
(12) Patent Application Publication
(10) Pub. No.: US 2023/0345821 A1
(43) Pub. Date: Oct. 26, 2023

(54) COMPOUND FOR ORGANIC ELECTRIC ELEMENT, ORGANIC ELECTRIC ELEMENT USING THE SAME, AND AN ELECTRONIC DEVICE THEREOF

(71) Applicant: DUK SAN NEOLUX CO., LTD., Chungcheongnam-do (KR)

(72) Inventors: Jun Seok SEO, Chungcheongnam-do (KR); Nam Geol LEE, Chungcheongnam-do (KR); Sun Hee LEE, Chungcheongnam-do (KR); Soung Yun MUN, Chungcheongnam-do (KR); Won Sam KIM, Chungcheongnam-do (KR); Hak Young LEE, Chungcheongnam-do (KR)

(21) Appl. No.: 18/018,315

(22) PCT Filed: Jul. 20, 2021
(86) PCT No.: PCT/KR2021/009300

§ 371 Date: Jan. 27, 2023

(30) Foreign Application Priority Data
Jul. 31, 2020 (KR) 10-2020-0096221

(51) Int. Cl. H10K 85/60; C07D 407/12; C09K 11/06; C07D 407/14; C07D 409/12

(52) U.S. Cl. CPC H10K 85/636; C07D 407/12; C07D 407/14; C07D 409/12; C09K 11/06; H10K 85/633; H10K 50/11

(57) ABSTRACT

A compound according to an embodiment is represented by Formula 1. An organic electric element includes a first electrode, a second electrode, and an organic material layer including the compound represented by Formula 1 between the first electrode and the second electrode. The driving voltage of the organic electric element can be lowered, and the luminous efficiency and life time can be improved.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2021/009300 filed on Jul. 20, 2021, which claims priority to the benefit of Korean Patent Application No. 10-2020-0096221 filed in the Korean Intellectual Property Office on Jul. 31, 2020, the entire contents of which are incorporated herein by reference.

### BACKGROUND

### 1. Technical Field

The present invention relates to organic electric element including organic compound and electronic device thereof.

### 2. Background Art

In general, an organic light emitting phenomenon refers to a phenomenon in which electric energy is converted into light energy of an organic material. An organic electric element utilizing the organic light emitting phenomenon usually has a structure including an anode, a cathode, and an organic material layer interposed therebetween. In many cases, the organic material layer has a multi-layered structure having respectively different materials in order to improve efficiency and stability of an organic electric element, and for example, may include a hole injection layer, a hole transport layer, a light emitting layer, an electron transport layer, an electron injection layer, or the like.

The most important issues in organic electroluminescent element are life and efficiency, and as the display becomes larger, these efficiency and life problems must be solved.

Efficiency, life span, driving voltage, and the like are correlated with each other. If efficiency is increased, then driving voltage is relatively lowered, and the crystallization of an organic material due to Joule heating generated during operation is reduced as driving voltage is lowered, as a result of which life span shows a tendency to increase.

However, efficiency cannot be maximized only by simply improving the organic material layer. This is because long life span and high efficiency can be simultaneously achieved when energy levels and T1 values among the respective layers included in the organic material layer, inherent material properties (mobility, interfacial properties, etc.) and the like are optimal combination.

In addition, an emission-auxiliary layer must be present between the hole transport layer and the light emitting layer in order to solve the problem of luminescence in the hole transport layer of recent organic electroluminescent devices, and material of different emission-auxiliary layers have been developed for each of the light emitting layers (R, G, B).

### SUMMARY

The object of the present invention is to provide a compound capable of lowering the driving voltage of the device and improving the luminous efficiency and life time, an organic electric element including the same, and an electronic device thereof.

In an aspect of the present invention, the present invention provides compound represented by the following formula.

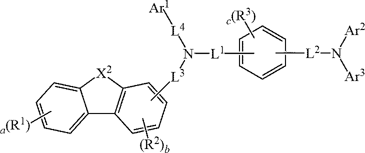

In another aspect of the present invention, the present invention provides an organic electric element employing compound represented by formula above and an electronic device thereof.

By using the compound according to embodiment of the present invention, a driving voltage of an organic electric element can be lowered and the luminous efficiency and lifetime of the element can be improved.

### BRIEF DESCRIPTION OF THE DRAWINGS

FIGS. 1 to 3 illustrate an example of organic electroluminescent element according to the present invention.

### DETAILED DESCRIPTION

Unless otherwise stated, the term “aryl group” or “arylene group” as used herein has, but not limited to, 6 to 60 carbon atoms. The aryl group or arylene group in the present invention may include a monocyclic ring, ring assemblies, a fused polycyclic system, spiro compound and the like. In addition, unless otherwise stated, a fluorenyl group may be included in an aryl group and a fluorenylene group may be included in an arylene group.

As used herein, the term “fluorenyl group” refers to a substituted or unsubstituted fluorenyl group, and “fluorenylene group” refers to a substituted or unsubstituted fluorenylene group. The fluorenyl group or fluorenylene group used in the present invention includes a spiro compound formed by combining R and R′ with each other in the following structure, and also includes compound formed by linking adjacent R″s to each other.

“Substituted fluorenyl group”, “substituted fluorenylene group” means that at least one of R, R′, R″ in the following structure is a substituent other than hydrogen, and R″ may be 1 to 8 in the following formula. In this specification, a fluorene group, a fluorenylene group, and a fluorenetriyl group may be referred to as a fluorene group regardless of the valence.

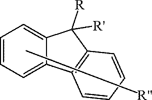

The term “spiro compound” as used herein has a spiro union which means union having one atom as the only common member of two rings. The common atom is designated as ‘spiro atom’. The compounds are defined as ‘monospiro-’, ‘dispiro-’ or ‘trispiro-’ depending on the number of spiro atoms in one compound.

The term “heterocyclic group” used in the specification includes a non-aromatic ring as well as an aromatic ring like “heteroaryl group” or “heteroarylene group”. Unless otherwise stated, the term “heterocyclic group” means, but not limited to, a ring containing one or more heteroatoms and having 2 to 60 carbon atoms. Unless otherwise stated, the term “heteroatom” as used herein represents, for example, N, O, S, P or Si and may include a heteroatom group such as SO₂, P=O etc. instead of carbon forming a ring such as the following compound. In the specification, “heterocyclic group” includes a monocyclic, ring assemblies, fused polycyclic system, a spiro-compound and the like.

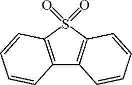 [SMILES: O=S1(=O)c2ccccc2-c2ccccc21]

The term “aliphatic ring group” as used herein refers to a cyclic hydrocarbon except for aromatic hydrocarbons, and includes a monocyclic ring, ring assemblies, a fused polycyclic system, a spiro-compound and the like, and unless otherwise specified, it means a ring of 3 to 60 carbon atoms, but not limited thereto. For example, a fused ring of benzene and cyclohexane corresponds to aliphatic ring group, wherein benzene is an aromatic ring and cyclohexane is a non-aromatic ring.

In this specification, a ‘group name’ corresponding to an aryl group, an arylene group, a heterocyclic group, and the like exemplified for each symbol and its substituent may be written in the name of functional group reflecting the valence, and may also be described as the name of a parent compound. For example, in the case of phenanthrene which is a kind of aryl group, it may be described by distinguishing valence such as ‘phenanthryl (group)’ when it is ‘monovalent group’, and as ‘phenanthrylene (group)’ when it is ‘divalent group’, and it may also be described as a parent compound name, ‘phenanthrene’, regardless of its valence. Similarly, in the case of pyrimidine, it may be described as ‘pyrimidine’ regardless of its valence, and it may also be described as the name of corresponding functional group such as pyrimidinyl (group) when it is ‘monovalent group’, and as ‘pyrimidylene (group)’ when it is ‘divalent group’.

In addition, in the present specification, the numbers and alphabets indicating a position may be omitted when describing a compound name or a substituent name, For example, pyrido[4,3-d]pyrimidine, benzopuro[2,3-d] pyrimidine and 9,9-dimethyl-9H-fluorene can be described as pyridopyrimidine, benzofurropyrimidine and dimethylfluorene, respectively. Therefore, both benzo[g]quinoxaline and benzo[f] quinoxaline can be described as benzoquinoxaline.

In addition, unless otherwise expressed, where any formula of the present invention is represented by the following formula, the substituent according to the index may be defined as follows.

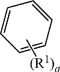

In the above formula, where a is an integer of zero, the substituent R¹ is absent, that is, hydrogen atoms are bonded to all the carbon constituting the benzene ring. Here, chemical formulas or compounds may be written without explicitly describing the hydrogen. In addition, one substituent R¹ is bonded to any carbon of the carbons forming the benzene ring when “a” is an integer of 1. Similarly, where “a” is an integer of 2 or 3, substituents R¹s may be bonded to the carbon of the benzene ring, for example, as followings. Also, where “a” is an integer of 4 to 6, substituents R¹s are bonded to the carbon of the benzene ring in a similar manner. Further, where “a” is an integer of 2 or more, R¹s may be the same or different from each other.

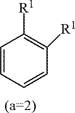

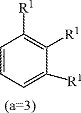

In addition, unless otherwise specified in the present specification, when referring to a condensed/fused ring, the number in the ‘number-condensed/fused ring’ indicates the number of condensed/fused rings. For example, a form in which three rings are condensed/fused with each other, such as anthracene, phenanthrene, and benzoquinazoline, may be represented by a 3-condensed/fused ring.

In addition, unless otherwise described herein, in the case of expressing a ring in the form of a ‘number-membered’ such as a 5-membered ring or a 6-membered ring, the number in the ‘number-membered’ represents the number of atoms forming the ring. For example, thiophene or furan may correspond to a 5-membered ring, and benzene or pyridine may correspond to a 6-membered ring.

In addition, unless otherwise specified in the present specification, the ring formed by adjacent groups may be selected from the group consisting of a C₆-C₆₀ aromatic ring group, a fluorenyl group, a C₂-C₆₀ heterocyclic group containing at least one heteroatom selected from the group consisting of O, N, S, Si and P, and a C₃-C₆₀ aliphatic ring.

Unless otherwise stated, the term “between adjacent groups”, for example, in case of the following Formulas, includes not only “between R₁ and R₂”, “between R₂ and R₃”, ” between R₃ and R₄”, “between R₅ and R₆”, but also “between R₇ and R₈” sharing one carbon, and may include “between substituents” attached to atom(carbon or nitrogen) consisting different ring, such as “between R₁ and R₇”, “between R₁ and R₈”, or “between R₄ and R₅” and the like. That is, where there are substituents bonded to adjacent elements constituting the same ring, the substituents may be correspond “adjacent groups”, and even if there are no adjacent substituents on the same ring, substituents attached to the adjacent ring may correspond to “adjacent groups”.

In the following Formula, when the substituents bonded to the same carbon, such as R₇ and R₈, are linked to each other to form a ring, a compound containing a spiro-moiety may be formed.

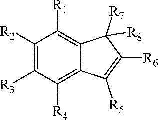

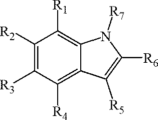

In addition, in the present specification, the expression ‘adjacent groups may be linked to each other to form a ring’ is used in the same sense as ‘adjacent groups are linked selectively to each other to form a ring’, and a case where at least one pair of adj acent groups may be bonded to each other to form a ring.

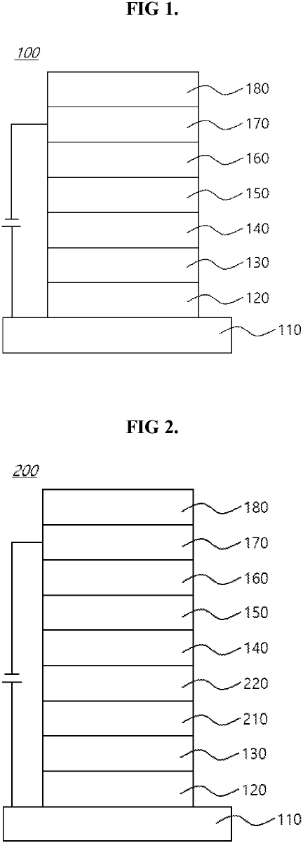

Hereinafter, referring to FIGS. 1 to 3, a lamination structure of an organic electric element including the compound of the present invention will be described.

In designation of reference numerals to components in respective drawings, it should be noted that the same elements will be designated by the same reference numerals although they are shown in different drawings. In addition, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used for defining an essence, order or sequence of a corresponding component but used merely to distinguish the corresponding component from other component(s). It will be understood that the expression “one component is “connected,” “coupled” or “joined” to another component “includes the case where a third component may be “connected,” “coupled,” and “joined” between the first and second components as well as the case where the first component may be directly connected, coupled or joined to the second component.

In addition, it will be understood that when an element such as a layer, film, region or substrate is referred to as being “on” or “over” another element, it can be directly on the other element or intervening elements may also be present. In contrast, when an element is referred to as being “directly on” another element, there are no intervening elements present.

The FIGS. 1 to 3 show an example of an organic electric element according to an embodiment of the present invention, respectively.

Referring to the FIG. 1, an organic electric element 100 according to an embodiment of the present invention includes a first electrode 110 formed on a substrate (not shown), a second electrode 170, and an organic material layer between the first electrode 110 and the second electrode 170.

The first electrode 110 may be an anode (positive electrode), and the second electrode 170 may be a cathode (negative electrode). In the case of an inverted organic electric element, the first electrode may be a cathode, and the second electrode may be an anode.

The organic material layer may be included a hole injection layer 120, a hole transport layer 130, a light emitting layer 140, an electron transport layer 150, and an electron injection layer 160. Specifically, a hole injection layer 120, a hole transport layer 130, a light emitting layer 140, an electron transport layer 150, and an electron injection layer 160 may be formed on the first electrode 110 in sequence.

Preferably, a layer for improving the luminous efficiency 180 may be formed one side of sides of the first electrode 110 and the second electrode 170, wherein one side is not facing the organic material layer, as a result the luminous efficiency of an organic electric element can be improved.

For example, the light efficiency improving layer 180 may be formed on the second electrode 170, as a result, in the case of a top emission organic light emitting diode, the optical energy loss due to Surface Plasmon Polaritons (SPPs) at the second electrode 170 may be reduced and in the case of a bottom emission organic light emitting diode, the light efficiency improving layer 180 may serve as a buffer for the second electrode 170.

A buffer layer 210 or an emission-auxiliary layer 220 may be further formed between the hole transport layer 130 and the light emitting layer 140, which will be described with reference to FIG. 2.

Referring to FIG. 2, the organic electric element 200 according to another embodiment of the present invention may include a hole injection layer 120, a hole transport layer 130, a buffer layer 210, an emission-auxiliary layer 220, a light emitting layer 140, the electron transport layer 150, the electron injection layer 160, and a second electrode 170 formed on a first electrode 110 in sequence, and a layer for improving light efficiency 180 may be formed on the second electrode 170.

Although not shown in FIG. 2, an electron transport auxiliary layer may be further formed between the light emitting layer 140 and the electron transport layer 150.

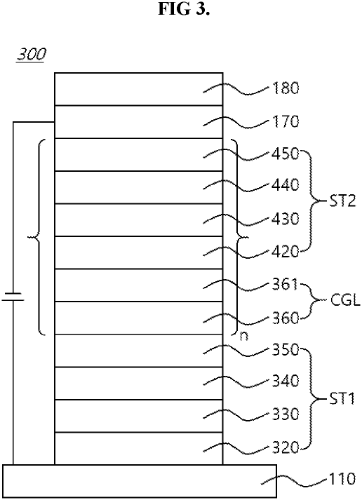

In addition, according to another embodiment of the present invention, the organic material layer may be a form consisting of a plurality of stacks, wherein the stacks include a hole transport layer, a light emitting layer, and an electron transport layer, respectively. This will be described with reference to FIG. 3.

Referring to FIG. 3, two or more sets of stacks of the organic material layers ST1 and ST2 may be formed between the first electrode 110 and the second electrode 170 in the organic electric element 300 according to another embodiment of the present invention, wherein the organic material layers are consisted of multiple layers, respectively, and the charge generation layer CGL may be formed between the stacks of the organic material layer.

Specifically, the organic electric element according to the embodiment of the present invention may include a first electrode 110, a first stack ST1, a charge generation layer CGL, a second stack ST2, and a second electrode 170 and a layer for improving light efficiency 180.

The first stack ST1 is an organic layer formed on the first electrode 110, and the first stack ST1 may include the first hole injection layer 320, the first hole transport layer 330, the first light emitting layer 340 and the first electron transport layer 350 and the second stack ST2 may include a second hole injection layer 420, a second hole transport layer 430, a second light emitting layer 440 and a second electron transport layer 450. As such, the first stack and the second stack may be the organic layers having the same or different stacked structures.

The charge generation layer CGL may be formed between the first stack ST1 and the second stack ST2. The charge generation layer CGL may include a first charge generation layer 360 and a second charge generation layer 361. The charge generating layer CGL is formed between the first light emitting layer 340 and the second light emitting layer 440 to increase the current efficiency generated in each light emitting layer and to smoothly distribute charges.

The first light emitting layer 340 may include a light emitting material including a blue host doped with a blue fluorescent dopant and the second light emitting layer 440 may include a light emitting material including a green host doped with a greenish yellow dopant and a red dopant together, but the material of the first light emitting layer 340 and the second light emitting layer 440 according to an embodiment of the present invention is not limited thereto.

In FIG. 3, n may be an integer of 1 to 5 and the charge generation layer CGL and the third stack may be further stacked on the second stack ST2 when n is 2.

When a plurality of light emitting layers are formed in a multi-layer stack structure as shown in FIG. 3, it is possible to manufacture an organic electroluminescent element that emits not only white light but also various colors, wherein the white light is emitted by the mixing effect of light emitted from each light emitting layer.

Compound represented by Formula 1 may be used as material of a hole injection layer 120, 320, 420, a hole transport layer 130, 330, 430, a buffer layer 210, an emission-auxiliary layer 220, an electron transport layer 150, 350, 450, an electron injection layer 160, a light emitting layer 140, 340, 440 or a layer for improving light efficiency 180, preferably, an emission-auxiliary layer 220.

Even if the cores of compounds are identical or similar to each other, the band gap, the electrical characteristics, the interface characteristics and the like may be different depending on which substituent is bonded at which position. Therefore, it is necessary to study the selection of the core and the combination with sub-substituent bonded to the core. In particular, long life span and high efficiency can be simultaneously achieved when the optimal combination of energy levels and T₁ values, inherent material properties (mobility, interfacial properties, etc.), and the like among the respective layers of an organic material layer is achieved.

Therefore, energy level and T₁ value between the respective layers of the organic material layer, inherent material properties (mobility, interfacial properties, etc.) and the like can be optimized by using compound represented by Formula 1 as material of an emission-auxiliary layer 220, and thus it is possible to simultaneously improve the lifetime and efficiency of the organic electric element.

The organic electric element according to an embodiment of the present invention may be manufactured using various deposition methods. The organic electric element according to an embodiment of the present invention may be manufactured using a PVD (physical vapor deposition) method or CVD (chemical vapor deposition) method. For example, the organic electric element may be manufactured by depositing a metal, a conductive metal oxide, or a mixture thereof on the substrate to form the anode 110, forming the organic material layer including the hole injection layer 120, the hole transport layer 130, the light emitting layer 140, the electron transport layer 150, and the electron injection layer 160 thereon, and then depositing a material, which can be used as the cathode 170, thereon. Also, an emission-auxiliary layer 220 may be formed between a hole transport layer 130 and a light emitting layer 140, and an electron transport auxiliary layer(not shown) may be further formed between a light emitting layer 140 and an electron transport layer 150 and, as described above, a stack structure may be formed.

Also, the organic material layer may be manufactured in such a manner that the fewer layers are formed using various polymer materials by a soluble process or solvent process, for example, spin coating, nozzle printing, inkjet printing, slot coating, dip coating, roll-to-roll, doctor blading, screen printing, or thermal transfer, instead of deposition. Since the organic material layer according to the present invention may be formed in various ways, the scope of protection of the present invention is not limited by a method of forming the organic material layer.

The organic electric element according to an embodiment of the present invention may be of a top emission type, a bottom emission type, or a dual emission type depending on the material used.

In addition, the organic electric element according to an embodiment of the present invention may be selected from the group consisting of an organic light emitting diode, an organic solar cell, an organic photo conductor, an organic transistor, an element for monochromatic illumination and an element for quantum dot display.

Another embodiment of the present invention provides an electronic device including a display device which includes the above described organic electric element, and a control unit for controlling the display device. Here, the electronic device may be a wired/wireless communication terminal which is currently used or will be used in the future, and covers all kinds of electronic devices including a mobile communication terminal such as a cellular phone, a personal digital assistant (PDA), an electronic dictionary, a point-to-multipoint (PMP), a remote controller, a navigation unit, a game player, various kinds of TVs, and various kinds of computers.

Hereinafter, compound according to one aspect of the present invention will be described.

Compound according to an aspect of the present invention is represented by Formula below.

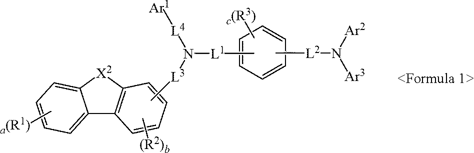

In Formula 1, each of symbols may be defined as follows.

Ar¹ is the following Formula A-1 or Formula A-2.

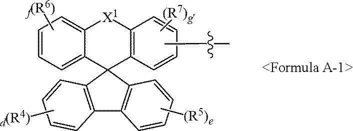

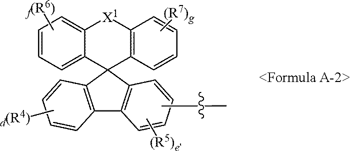

Ar² and Ar³ are each independently selected from the group consisting of a C₆-C₆₀ aryl group, a fluorenyl group, a C₂-C₆₀ heterocyclic group containing at least one heteroatom of O, N, S, Si and P, and a C₃-C₆₀ aliphatic ring group.

X¹ is O or S, and X² is N(R′), O or S.

R¹ to R⁷ are each independently selected from the group consisting of hydrogen, deuterium, halogen, a cyano group, a nitro group, a C₆-C₆₀ aryl group, a fluorenyl group, a C₂-C₆₀ heterocyclic group including at least one heteroatom selected from the group consisting of O, N, S, Si and P, a C₃-C₆₀ aliphatic ring group, a C₁-C₃₀ alkyl group, a C₂-C₃₀ alkenyl group, a C₂-C₃₀ alkynyl group, a C₁-C₃₀ alkoxyl group, and a C₆-C₃₀ aryloxy group, and adjacent groups may be bonded to each other to form a ring.

a, c, d, e, f and g are each an integer of 0 to 4, b, e′ and g′ are each an integer of 0 to 3, and where each of these is an integer of 2 or more, each of R¹s, each of R²s, each of R³s, each of R⁴s, each of R⁵s, each of R⁶s, each of R⁷s are the same as or different from each other.

The ring formed by at least one pair of adjacent R¹s, adjacent R²s, adjacent R³s, adjacent R⁴s, adjacent R⁵s, adjacent R⁶s and adjacent R⁷s may be selected from the group consisting of a C₆-C₆₀ aromatic ring group, a fluorene group, a C₂-C₆₀ heterocyclic group containing at least one heteroatom of O, N, S, Si and P, and a C₃-C₆₀ aliphatic ring group.

When an aromatic ring group is formed by adjacent groups, the aromatic ring group may be a C₆-C₃₀, a C₆-C₂₀, a C₆-C₁₆, a C₆-C₁₄, a C₆-C₁₀ or a C₆ aromatic ring group, specifically, benzene, naphthalene, phenanthrene or the like.

When a heterocyclic group is formed by adjacent groups, the heterocyclic group may be, for example, a C₂-C₃₀, a C₃-C₃₀, a C₄-C₃₀, a C₄-C₂₅, a C₄-C₂₄, a C₄-C₂₀, a C₄-C₁₈, a C₄-C₁₆, a C₄-C₁₂, a C₄-C₁₁, a C₄-C₁₀, a C₄-C₉, a C₄-C₈, a C₄-C₇, a C₄-C₆, a C₄-C₅, a C₄, a C₅, a C₆, a C₇, a C₈, a C₉, a C₁₀, a C₁₂, a C₁₆ heterocyclic group, specifically, furan, thiophene, benzofuran, benzothiophene, dibenzofuran, dibenzothiophene or the like.

For example, when adjacent R³s are linked to each other to form a ring, compound represented by the following Formula may be formed together with phenylene to which R³s are bonded. That is, when adjacent R³s are bonded to each other to form a 2-condensed ring such as benzothiophene or benzofuran, a 3-condensed ring such as dibenzothiophene or dibenzofuran may eventually be formed.

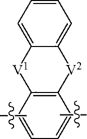

Wherein, V¹ and V² are the same as defined in Formula 1-7 to Formula 1-18.

L¹ to L⁴ are each independently selected from the group consisting of a single bond, a C₆-C₆₀ arylene group, a fluorenylene group, a C₃-C₆₀ aliphatic ring group, and a C₂-C₆₀ heterocyclic group containing at least one heteroatom of O, N, S, Si and P.

R′ is selected from the group consisting of a C₆-C₆₀ aryl group, a fluorenyl group, a C₂-C₆₀ heterocyclic group containing at least one heteroatom of O, N, S, Si and P and a C₃-C₆₀ aliphatic ring group.

When at least one of Ar², Ar³, R¹ to R⁷ and R′ is an aryl group, the aryl group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, or a C₁₈ aryl group, specifically, phenyl, biphenyl, naphthyl, terphenyl, phenanthrene, triphenylene, or the like.

When at least one of L¹ to L⁴ is an arylene group, the arylene group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, a C₁₈ aryl group, specifically, phenylene, biphenyl, naphthylene, terphenyl, phenanthrene or the like.

When at least one of Ar², Ar³, R¹ to R⁷, R′ and L¹ to L⁴ is a heterocyclic group, the heterocyclic group may be, for example, a C₂-C₃₀, a C₂-C₂₉, a C₂-C₂₈, a C₂-C₂₇, a C₂-C₂₆, a C₂-C₂₅, a C₂-C₂₄, a C₂-C₂₃, a C₂-C₂₂, a C₂-C₂₁, a C₂-C₂₀, a C₂-C₁₉, a C₂-C₁₈, a C₂-C₁₇, a C₂-C₁₆, a C₂-C₁₅, a C₂-C₁₄, a C₂-C₁₃, a C₂-C₁₂, a C₂-C₁₁, a C₂-C₁₀, a C₂-C₉, a C₂-C₈, a C₂-C₇, a C2-C6, a C₂-C₅, a C₂-C₄, a C₂-C₃, a C₂, a C₃, a C₄, a C₅, a C₆, a C₇, a C₈, a C₉, a C₁₀, a C₁₁, a C₁₂, a C₁₃, a C₁₄, a C₁₅, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, or a C₂₉ heterocyclic group, specifically, pyridine, pyrimidine, pyrazine, pyridazine, triazine, furan, pyrrole, silole, indene, indole, phenyl-indole, benzoindole, phenyl-benzoindole, pyrazinoindol, quinoline, isoquinoline, benzoquinoline, pyridoquinoline, quinazoline, benzoquinazoline, dibenzoquinazoline, phenanthroquinazoline, quinoxaline, benzoquinoxaline, dibenzoquinoxaline, benzofuran, naphthobenzofuran, dibenzofuran, phenanthrobenzofuran, dinaphthofuran, thiophene, benzothiophene, dibenzothiophene, naphthobenzothiophene, phenanthrobenzothiophene, dinaphthothiophene, carbazole, phenyl-carbazole, benzocarbazole, phenyl-benzocarbazole, naphthyl-benzocarbazole, dibenzocarbazole, indolocarbazole, benzofuropyridine, benzothienopyridine, benzofuropyridine, benzothienopyrimidine, benzofuropyrimidine, benzothienopyrazine, benzofuropyrazine, benzoimidazole, benzothiazole, benzooxazole, benzosiloe, phenanthroline, dihydro-phenylphenazine, 10-phenyl-10H-phenoxazine, phenoxazine, phenothiazine, dibenzodioxin, benzodibenzodioxin, thianthrene, 9,9-dimethyl-9H-xantene, 9,9-dimethyl-9H-thioxantene, dihydrodimethylphenylacridine, spiro[fluorene-9,9′-xanthene] and the like.

When at least one of Ar², Ar³, R¹ to R⁷ and R′ is a fluorenyl group or at least one of L¹ to L⁴ is a fluorenylene group, the fluorenyl group or the fluorenylene group may be, for example, 9,9-dimethyl-9H-fluorene, 9,9-diphenyl-9H-fluorene, 9,9′-spirobifluorene, spiro[benzo[b]fluorene-11,9′-fluorene], benzo[b]fluorene, 11,11-diphenyl-11H-benzo[b]fluorene, 9-(naphthalen-2-yl)9-phenyl-9H-fluorene, and the like.

When at least one of Ar², Ar³, R¹ to R⁷, R′, L¹ to L⁴ is an aliphatic ring group, the aliphatic ring group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, a C₁₈ aliphatic ring group.

The aryl group, the arylene group, the fluorenyl group, the fluorenylene group, the heterocyclic group, the aliphatic ring group, the alkyl group, the alkenyl group, the alkynyl group, the alkoxyl group, the aryloxyl group, and the ring formed by adjacent groups may be each optionally substituted with one or more substituents selected from the group consisting of deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group.

When at least one of the aryl group, the fluorenyl group, the heterocyclic group, the aliphatic ring group, the alkyl group, the alkenyl group, the alkynyl group, the alkoxyl group, the aryloxyl group, the arylene group, the fluorenylene group, and the ring formed by adjacent groups is substituted with an aryl group, the aryl group may be, for example, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇ or a C₁₈ aryl group.

When at least one of the aryl group, the fluorenyl group, the heterocyclic group, the aliphatic ring group, the alkyl group, the alkenyl group, the alkynyl group, the alkoxyl group, the aryloxyl group, the arylene group, the fluorenylene group, and the ring formed by adjacent groups is substituted with a heterocyclic group, the heterocyclic group may be, for example, a C₂-C₂₀, a C₂-C₁₉, a C₂-C₁₈, a C₂-C₁₇, a C₂-C₁₆, a C₂-C₁₅, a C₂-C₁₄, a C₂-C₁₃, a C₂-C₁₂, a C₂-C₁₁, a C₂-C₁₀, a C₂-C₉, a C₂-C₈, a C₂-C₇, a C₂-C₆, a C₂-C₅, a C₂-C₄, a C₂-C₃, a C₂, a C₃, a C₄, a C₅, a C₆, a C₇, a C₈, a C₉, a C₁₀, a C₁₁, a C₁₂, a C₁₃, a C₁₄, a C₁₅, a C₁₆, a C₁₇, a C₁₈, a C₁₉ or a C₂₀ heterocyclic group.

When at least one of the aryl group, the fluorenyl group, the heterocyclic group, the aliphatic ring group, the alkyl group, the alkenyl group, the alkynyl group, the alkoxyl group, the aryloxyl group, the arylene group, the fluorenylene group, and the ring formed by adjacent groups is substituted with an aliphatic ring group, the aliphatic ring group may be, for example, a C₃-C₂₀, a C₃-C₁₉, a C₃-C₁₈, a C₃-C₁₇, a C₃-C₁₆, a C₃-C₁₅, a C₃-C₁₄, a C₃-C₁₃, a C₃-C₁₂, a C₃-C₁₁, a C₃-C₁₀, a C₃-C₆, a C₆, a C₁₀, a C₁₂, a C₁₃, a C₁₄, a C₁₅, a C₁₆, a C₁₇, a C₁₈ aliphatic ring group.

When at least one of the aryl group, the fluorenyl group, the heterocyclic group, the aliphatic ring group, the alkyl group, the alkenyl group, the alkynyl group, the alkoxyl group, the aryloxyl group, the arylene group, the fluorenylene group, and the ring formed by adjacent groups is substituted with a fluorenyl group, the fluorenyl group may be 9-dimethyl-9H-fluorene, 9,9-diphenyl-9H-fluorene, 9,9′-spirobifluorene, spiro[benzo[b]fluorene-11,9′-fluorene], benzo[b]fluorene, 11,11-diphenyl-11H-benzo[b]fluorene, 9-(naphthalen-2-yl)9-phenyl-9H-fluorene, and the like.

Formula 1 may be represented by one of the following Formula 1-1 to Formula 1-18.

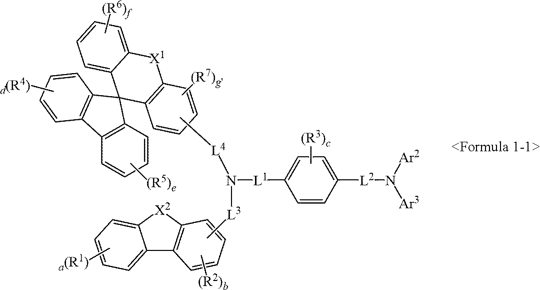

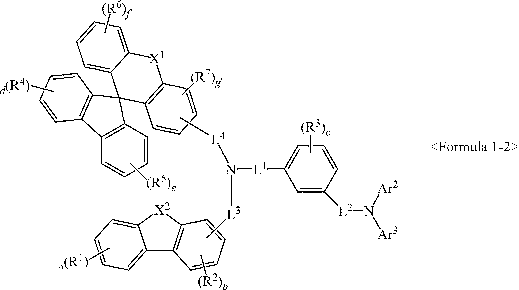

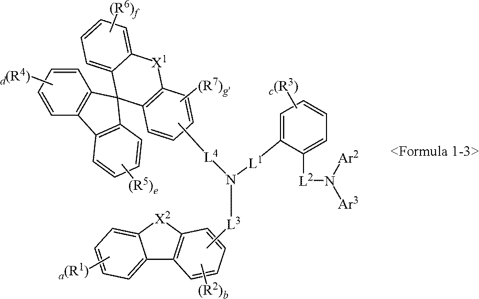

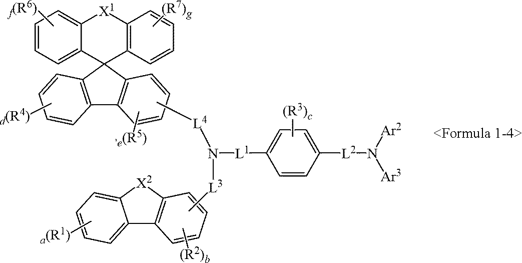

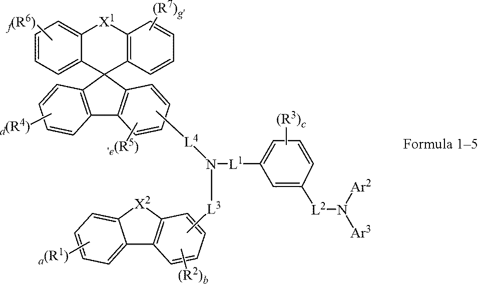

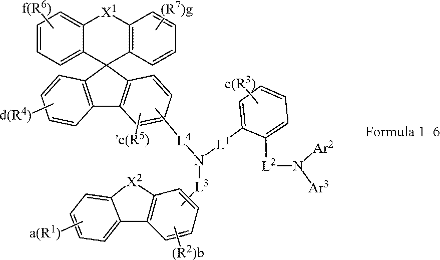

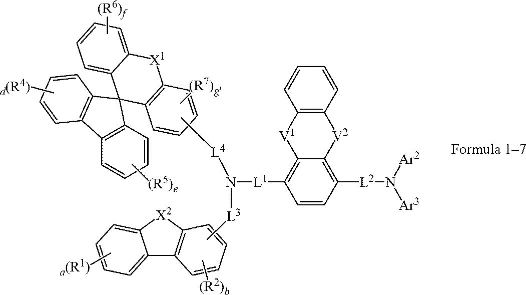

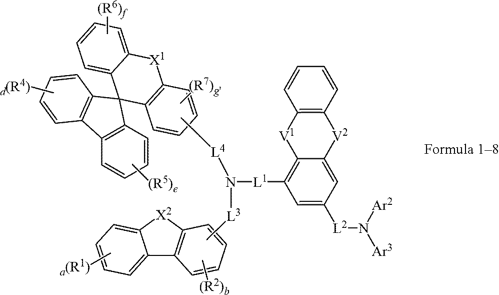

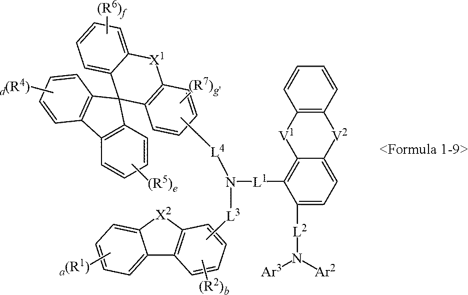

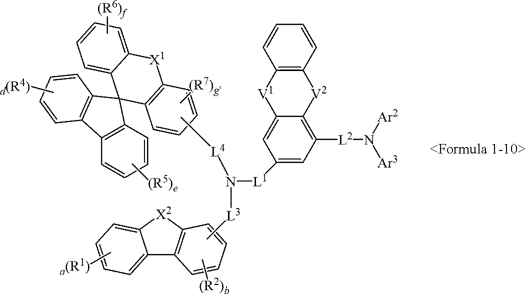

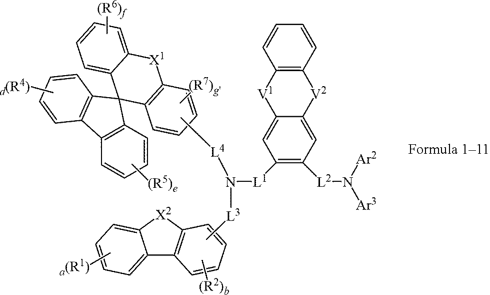

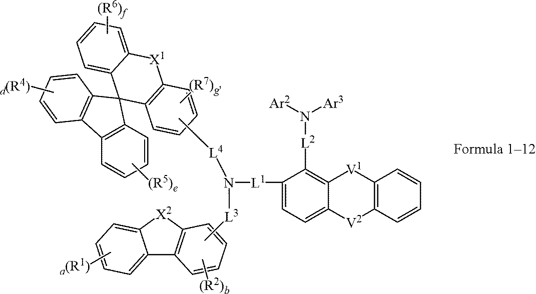

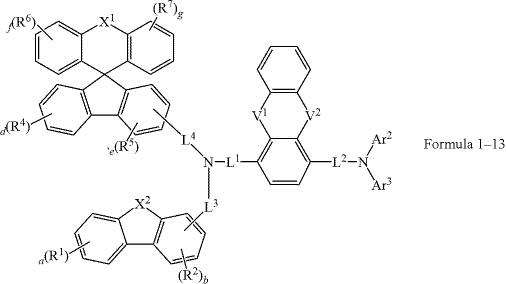

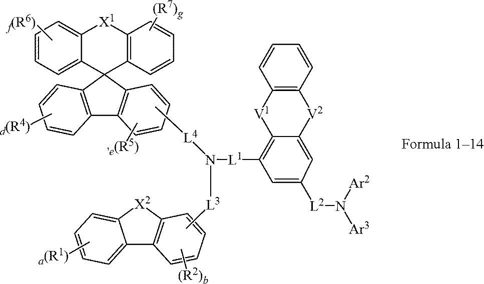

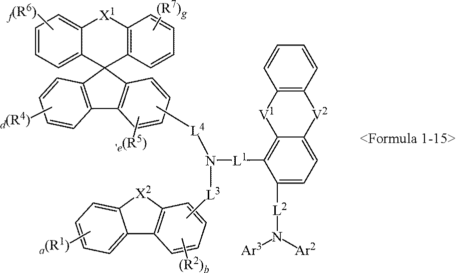

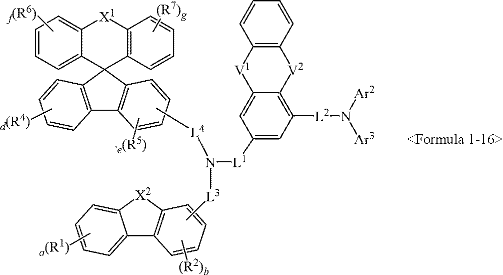

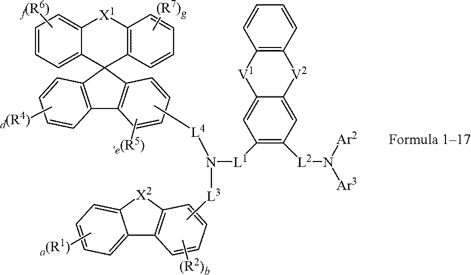

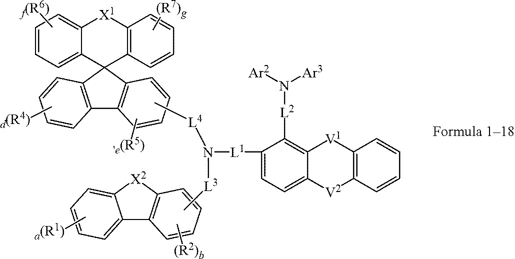

In Formula 1-1 to Formula 1-18, X¹, X², L¹ to L⁴, Ar², Ar³, R¹ to R⁷ and a to g are the same as defined in Formula 1, and e′ and g′ are each an integer of 0 to 3,

V¹ and V² are each independently a single bond, O, S, C(R₁)(R₂) or N(R₃), and the case where both V¹ and V² are a single bond is excluded.

R₁ and R₂ are each independently selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group, and R₁ and R₂ may be linked to each other to form a ring.

R₃ is selected from the group consisting of a C₆-C₂₀ aryl group, a fluorenyl group, a C₂-C₂₀ heterocyclic group containing at least one heteroatom of O, N, S, Si and P, a C₃-C₂₀ aliphatic ring group, a C₁-C₂₀ alkoxyl group, a C₆-C₂₀ aryloxy group, a C₁-C₂₀ alkyl group, a C₂-C₂₀ alkenyl group, and a C₂-C₂₀ alkynyl group.

In the above Formula, at least one of Ar² and Ar³ may be selected from the group consisting of Formula 2-1 to Formula 2-6.

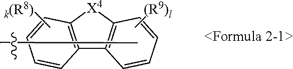

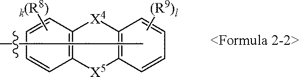

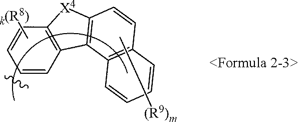

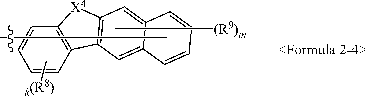

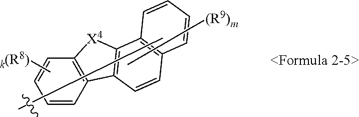

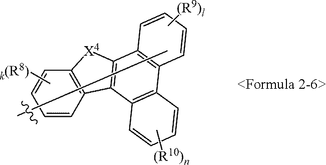

In Formula 2-1 to Formula 2-6, each of symbols may be defined as follows.

X⁴ and X⁵ are each independently O, S, C(R₁)(R₂) or N(R₃).

R₁, R₂, R⁸, R⁹ and R¹⁰ are each independently selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group, adjacent groups may be linked to each other to form a ring, and R₁ and R₂ may be linked to each other to form a ring.

k, l and n are each an integer of 0 to 4, m is an integer of 0 to 6, and where each of these is an integer of 2 or more, each of R⁸s, each of R⁹s, each of R¹⁰s are the same as or different from each other.

R₃ is selected from the group consisting of a C₆-C₂₀ aryl group, a fluorenyl group, a C₂-C₂₀ heterocyclic group containing at least one heteroatom of O, N, S, Si and P, a C₃-C₂₀ aliphatic ring group, a C₁-C₂₀ alkoxyl group, a C₆-C₂₀ aryloxy group, a C₁-C₂₀ alkyl group, a C₂-C₂₀ alkenyl group, and a C₂-C₂₀ alkynyl group.

Specifically, the compound represented by Formula 1 may be one of the following compounds, but there is no limitation thereto.

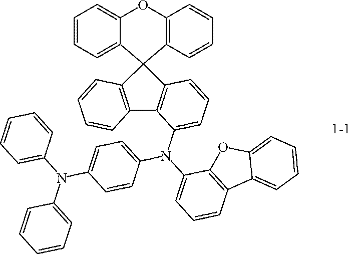 [SMILES: c1ccc(N(c2ccccc2)c2ccc(N(c3cccc4c3-c3ccccc3C43c4ccccc4Oc4ccccc43)c3cccc4c3oc3ccccc34)cc2)cc1]

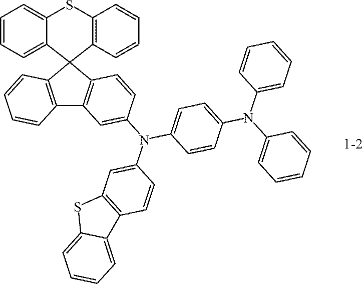 [SMILES: c1ccc(N(c2ccccc2)c2ccc(N(c3ccc4c(c3)-c3ccccc3C43c4ccccc4Sc4ccccc43)c3ccc4c(c3)sc3ccccc34)cc2)cc1]

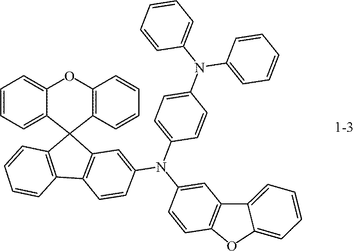 [SMILES: c1ccc(N(c2ccccc2)c2ccc(N(c3ccc4c(c3)C3(c5ccccc5Oc5ccccc53)c3ccccc3-4)c3ccc4oc5ccccc5c4c3)cc2)cc1]

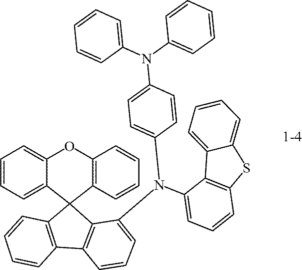 [SMILES: c1ccc(N(c2ccccc2)c2ccc(N(c3cccc4c3C3(c5ccccc5Oc5ccccc53)c3ccccc3-4)c3cccc4sc5ccccc5c34)cc2)cc1]

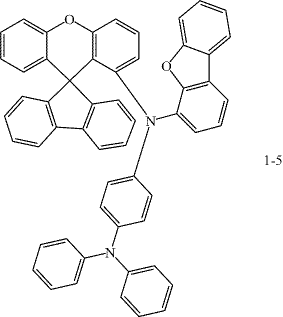 [SMILES: c1ccc(N(c2ccccc2)c2ccc(N(c3cccc4c3C3(c5ccccc5O4)c4ccccc4-c4ccccc43)c3cccc4c3oc3ccccc34)cc2)cc1]

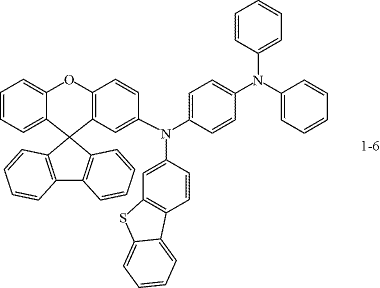 [SMILES: c1ccc(N(c2ccccc2)c2ccc(N(c3ccc4c(c3)C3(c5ccccc5O4)c4ccccc4-c4ccccc43)c3ccc4c(c3)sc3ccccc34)cc2)cc1]

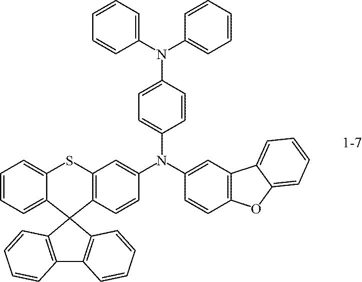 [SMILES: c1ccc(N(c2ccccc2)c2ccc(N(c3ccc4c(c3)Sc3ccccc3C43c4ccccc4-c4ccccc43)c3ccc4oc5ccccc5c4c3)cc2)cc1]

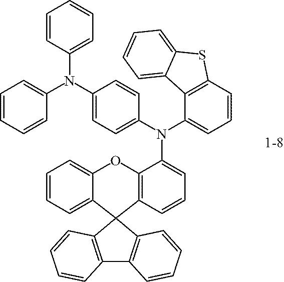 [SMILES: c1ccc(N(c2ccccc2)c2ccc(N(c3cccc4c3Oc3ccccc3C43c4ccccc4-c4ccccc43)c3cccc4sc5ccccc5c34)cc2)cc1]

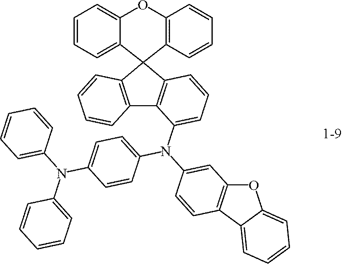 [SMILES: c1ccc(N(c2ccccc2)c2ccc(N(c3ccc4c(c3)oc3ccccc34)c3cccc4c3-c3ccccc3C43c4ccccc4Oc4ccccc43)cc2)cc1]

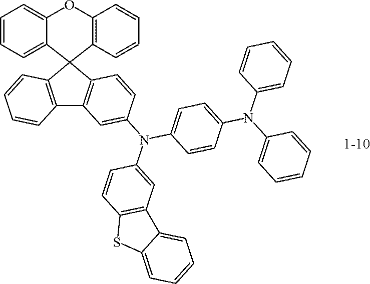 [SMILES: c1ccc(N(c2ccccc2)c2ccc(N(c3ccc4c(c3)-c3ccccc3C43c4ccccc4Oc4ccccc43)c3ccc4sc5ccccc5c4c3)cc2)cc1]

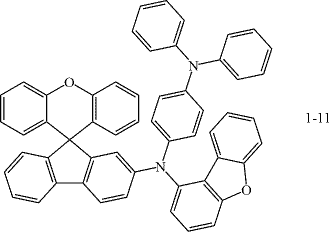 [SMILES: c1ccc(N(c2ccccc2)c2ccc(N(c3ccc4c(c3)C3(c5ccccc5Oc5ccccc53)c3ccccc3-4)c3cccc4oc5ccccc5c34)cc2)cc1]

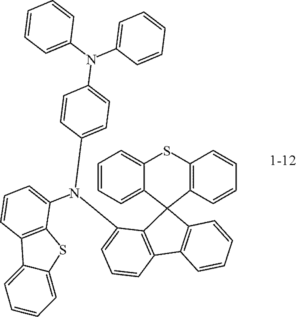 [SMILES: c1ccc(N(c2ccccc2)c2ccc(N(c3cccc4c3C3(c5ccccc5Sc5ccccc53)c3ccccc3-4)c3cccc4c3sc3ccccc34)cc2)cc1]

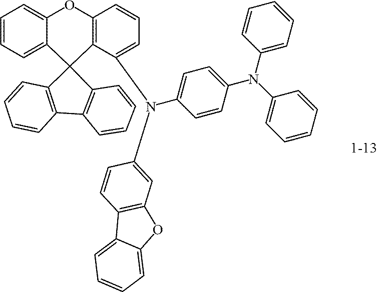 [SMILES: c1ccc(N(c2ccccc2)c2ccc(N(c3ccc4c(c3)oc3ccccc34)c3cccc4c3C3(c5ccccc5O4)c4ccccc4-c4ccccc43)cc2)cc1]

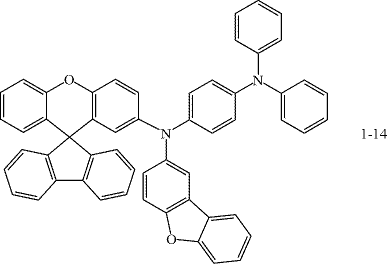 [SMILES: c1ccc(N(c2ccccc2)c2ccc(N(c3ccc4c(c3)C3(c5ccccc5O4)c4ccccc4-c4ccccc43)c3ccc4oc5ccccc5c4c3)cc2)cc1]

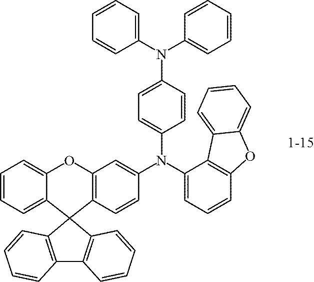 [SMILES: c1ccc(N(c2ccccc2)c2ccc(N(c3ccc4c(c3)Oc3ccccc3C43c4ccccc4-c4ccccc43)c3cccc4oc5ccccc5c34)cc2)cc1]

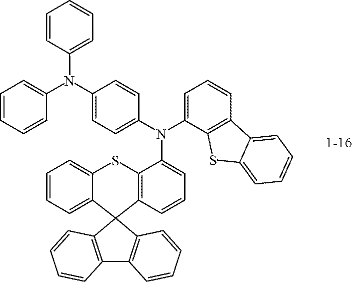 [SMILES: c1ccc(N(c2ccccc2)c2ccc(N(c3cccc4c3Sc3ccccc3C43c4ccccc4-c4ccccc43)c3cccc4c3sc3ccccc34)cc2)cc1]

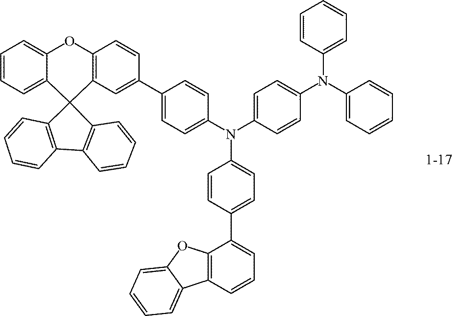 [SMILES: c1ccc(N(c2ccccc2)c2ccc(N(c3ccc(-c4ccc5c(c4)C4(c6ccccc6O5)c5ccccc5-c5ccccc54)cc3)c3ccc(-c4cccc5c4oc4ccccc45)cc3)cc2)cc1]

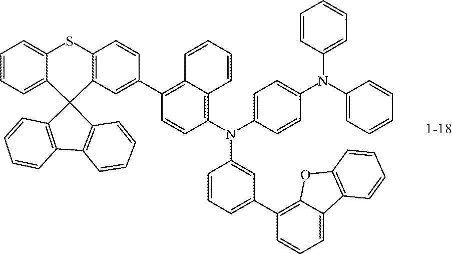 [SMILES: c1ccc(N(c2ccccc2)c2ccc(N(c3cccc(-c4cccc5c4oc4ccccc45)c3)c3ccc(-c4ccc5c(c4)C4(c6ccccc6S5)c5ccccc5-c5ccccc54)c4ccccc34)cc2)cc1]

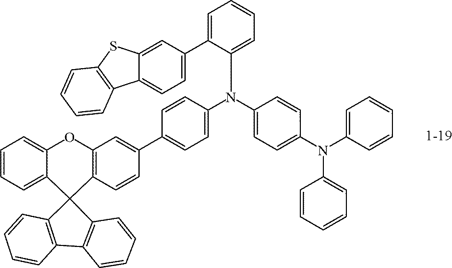 [SMILES: c1ccc(N(c2ccccc2)c2ccc(N(c3ccc(-c4ccc5c(c4)Oc4ccccc4C54c5ccccc5-c5ccccc54)cc3)c3ccccc3-c3ccc4c(c3)sc3ccccc34)cc2)cc1]

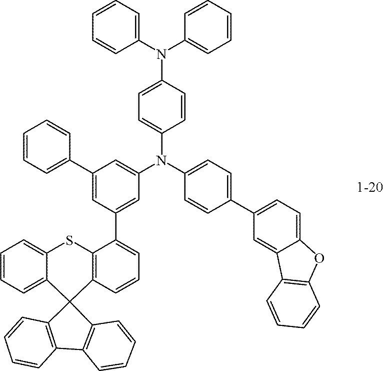 [SMILES: c1ccc(-c2cc(-c3cccc4c3Sc3ccccc3C43c4ccccc4-c4ccccc43)cc(N(c3ccc(-c4ccc5oc6ccccc6c5c4)cc3)c3ccc(N(c4ccccc4)c4ccccc4)cc3)c2)cc1]

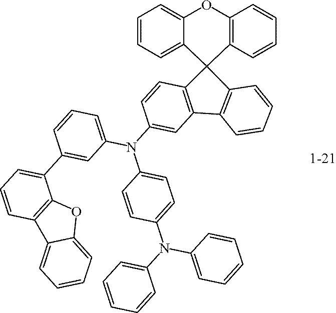 [SMILES: c1ccc(N(c2ccccc2)c2ccc(N(c3cccc(-c4cccc5c4oc4ccccc45)c3)c3ccc4c(c3)-c3ccccc3C43c4ccccc4Oc4ccccc43)cc2)cc1]

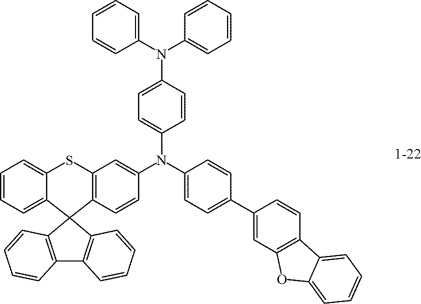 [SMILES: c1ccc(N(c2ccccc2)c2ccc(N(c3ccc(-c4ccc5c(c4)oc4ccccc45)cc3)c3ccc4c(c3)Sc3ccccc3C43c4ccccc4-c4ccccc43)cc2)cc1]

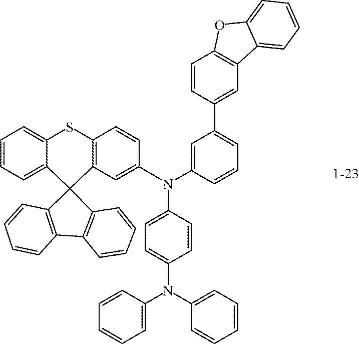 [SMILES: c1ccc(N(c2ccccc2)c2ccc(N(c3cccc(-c4ccc5oc6ccccc6c5c4)c3)c3ccc4c(c3)C3(c5ccccc5S4)c4ccccc4-c4ccccc43)cc2)cc1]

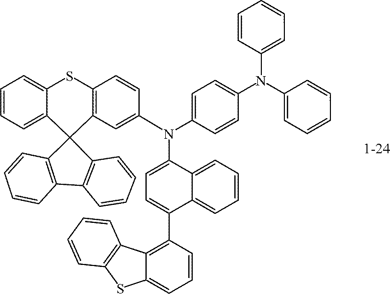 [SMILES: c1ccc(N(c2ccccc2)c2ccc(N(c3ccc4c(c3)C3(c5ccccc5S4)c4ccccc4-c4ccccc43)c3ccc(-c4cccc5sc6ccccc6c45)c4ccccc34)cc2)cc1]

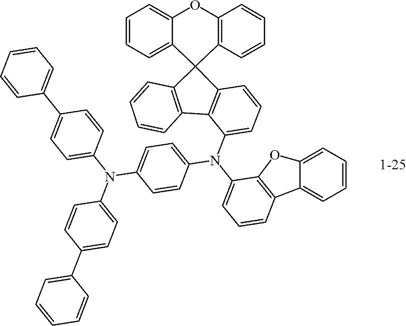 [SMILES: c1ccc(-c2ccc(N(c3ccc(-c4ccccc4)cc3)c3ccc(N(c4cccc5c4-c4ccccc4C54c5ccccc5Oc5ccccc54)c4cccc5c4oc4ccccc45)cc3)cc2)cc1]

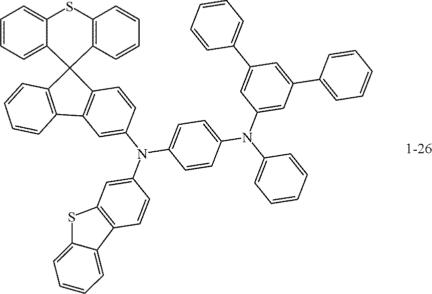 [SMILES: c1ccc(-c2cc(-c3ccccc3)cc(N(c3ccccc3)c3ccc(N(c4ccc5c(c4)-c4ccccc4C54c5ccccc5Sc5ccccc54)c4ccc5c(c4)sc4ccccc45)cc3)c2)cc1]

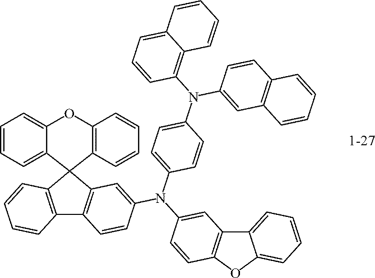 [SMILES: c1ccc2c(c1)Oc1ccccc1C21c2ccccc2-c2ccc(N(c3ccc(N(c4ccc5ccccc5c4)c4cccc5ccccc45)cc3)c3ccc4oc5ccccc5c4c3)cc21]

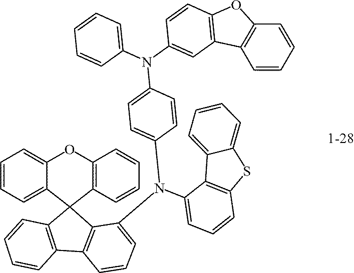 [SMILES: c1ccc(N(c2ccc(N(c3cccc4c3C3(c5ccccc5Oc5ccccc53)c3ccccc3-4)c3cccc4sc5ccccc5c34)cc2)c2ccc3oc4ccccc4c3c2)cc1]

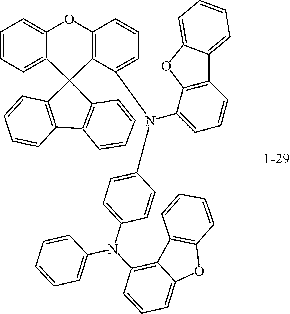 [SMILES: c1ccc(N(c2ccc(N(c3cccc4c3C3(c5ccccc5O4)c4ccccc4-c4ccccc43)c3cccc4c3oc3ccccc34)cc2)c2cccc3oc4ccccc4c23)cc1]

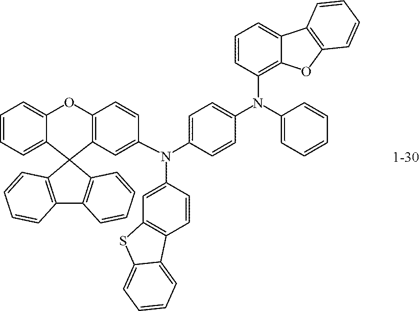 [SMILES: c1ccc(N(c2ccc(N(c3ccc4c(c3)C3(c5ccccc5O4)c4ccccc4-c4ccccc43)c3ccc4c(c3)sc3ccccc34)cc2)c2cccc3c2oc2ccccc23)cc1]

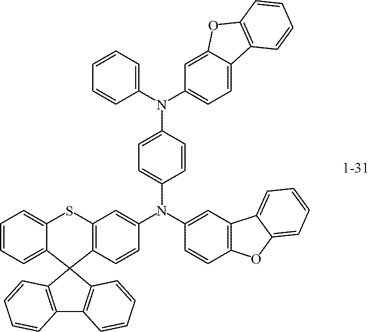 [SMILES: c1ccc(N(c2ccc(N(c3ccc4c(c3)Sc3ccccc3C43c4ccccc4-c4ccccc43)c3ccc4oc5ccccc5c4c3)cc2)c2ccc3c(c2)oc2ccccc23)cc1]

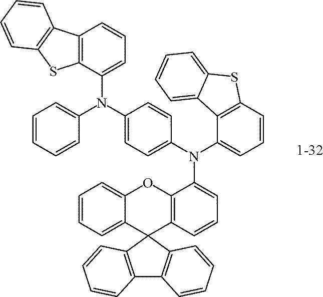 [SMILES: c1ccc(N(c2ccc(N(c3cccc4c3Oc3ccccc3C43c4ccccc4-c4ccccc43)c3cccc4sc5ccccc5c34)cc2)c2cccc3c2sc2ccccc23)cc1]

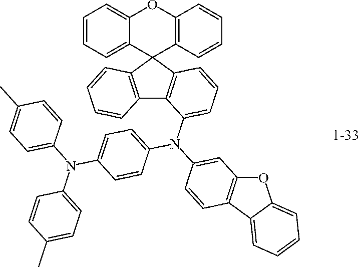 [SMILES: Cc1ccc(N(c2ccc(C)cc2)c2ccc(N(c3ccc4c(c3)oc3ccccc34)c3cccc4c3-c3ccccc3C43c4ccccc4Oc4ccccc43)cc2)cc1]

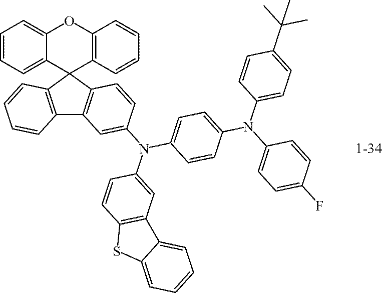 [SMILES: CC(C)(C)c1ccc(N(c2ccc(F)cc2)c2ccc(N(c3ccc4c(c3)-c3ccccc3C43c4ccccc4Oc4ccccc43)c3ccc4sc5ccccc5c4c3)cc2)cc1]

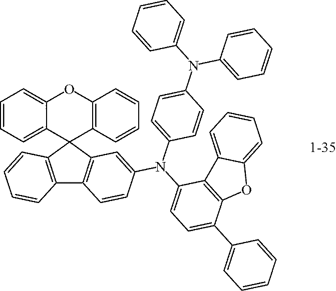 [SMILES: c1ccc(-c2ccc(N(c3ccc(N(c4ccccc4)c4ccccc4)cc3)c3ccc4c(c3)C3(c5ccccc5Oc5ccccc53)c3ccccc3-4)c3c2oc2ccccc23)cc1]

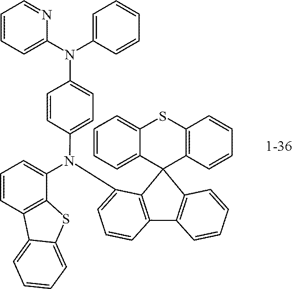 [SMILES: c1ccc(N(c2ccc(N(c3cccc4c3C3(c5ccccc5Sc5ccccc53)c3ccccc3-4)c3cccc4c3sc3ccccc34)cc2)c2ccccn2)cc1]

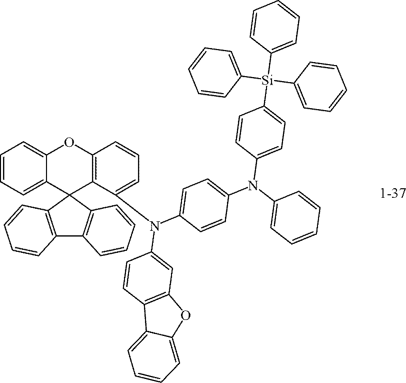 [SMILES: c1ccc(N(c2ccc(N(c3ccc4c(c3)oc3ccccc34)c3cccc4c3C3(c5ccccc5O4)c4ccccc4-c4ccccc43)cc2)c2ccc([Si](c3ccccc3)(c3ccccc3)c3ccccc3)cc2)cc1]

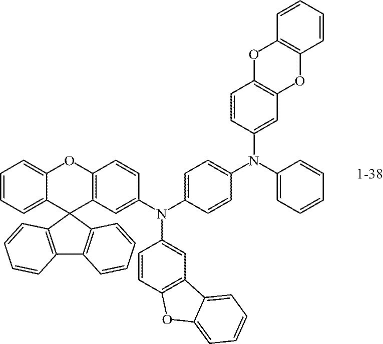 [SMILES: c1ccc(N(c2ccc(N(c3ccc4c(c3)C3(c5ccccc5O4)c4ccccc4-c4ccccc43)c3ccc4oc5ccccc5c4c3)cc2)c2ccc3c(c2)Oc2ccccc2O3)cc1]

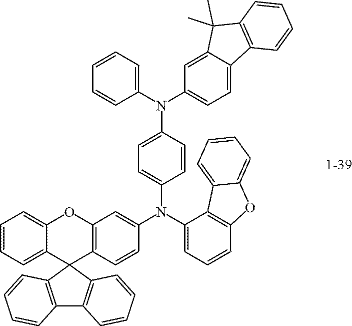 [SMILES: CC1(C)c2ccccc2-c2ccc(N(c3ccccc3)c3ccc(N(c4ccc5c(c4)Oc4ccccc4C54c5ccccc5-c5ccccc54)c4cccc5oc6ccccc6c45)cc3)cc21]

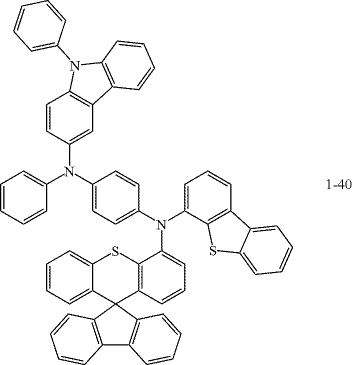 [SMILES: c1ccc(N(c2ccc(N(c3cccc4c3Sc3ccccc3C43c4ccccc4-c4ccccc43)c3cccc4c3sc3ccccc34)cc2)c2ccc3c(c2)c2ccccc2n3-c2ccccc2)cc1]

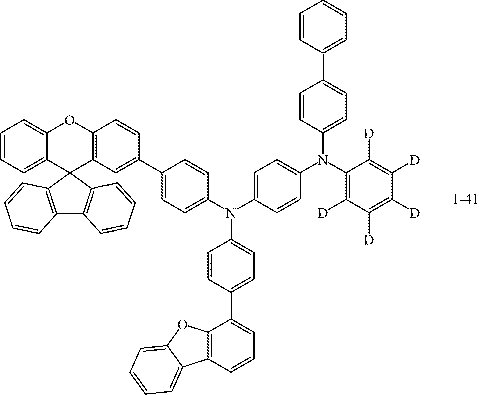 [SMILES: [2H]c1c([2H])c([2H])c(N(c2ccc(-c3ccccc3)cc2)c2ccc(N(c3ccc(-c4ccc5c(c4)C4(c6ccccc6O5)c5ccccc5-c5ccccc54)cc3)c3ccc(-c4cccc5c4oc4ccccc45)cc3)cc2)c([2H])c1[2H]]

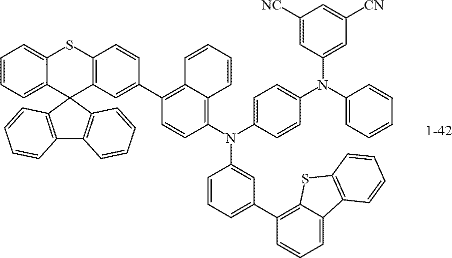

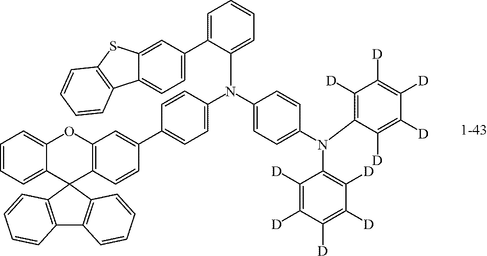 [SMILES: [2H]c1c([2H])c([2H])c(N(c2ccc(N(c3ccc(-c4ccc5c(c4)Oc4ccccc4C54c5ccccc5-c5ccccc54)cc3)c3ccccc3-c3ccc4c(c3)sc3ccccc34)cc2)c2c([2H])c([2H])c([2H])c([2H])c2[2H])c([2H])c1[2H]]

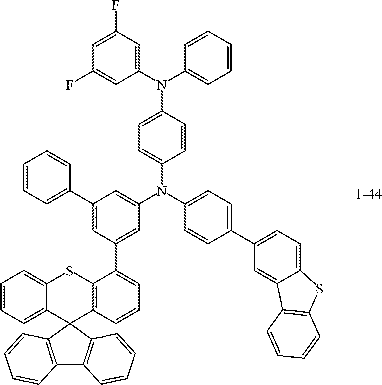 [SMILES: Fc1cc(F)cc(N(c2ccccc2)c2ccc(N(c3ccc(-c4ccc5sc6ccccc6c5c4)cc3)c3cc(-c4ccccc4)cc(-c4cccc5c4Sc4ccccc4C54c5ccccc5-c5ccccc54)c3)cc2)c1]

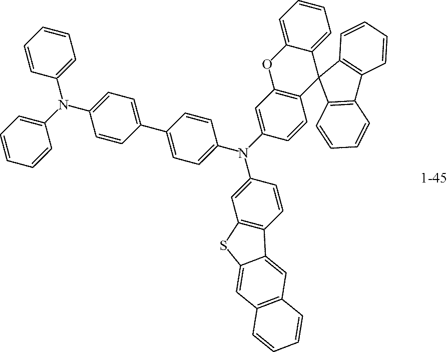 [SMILES: c1ccc(N(c2ccccc2)c2ccc(-c3ccc(N(c4ccc5c(c4)Oc4ccccc4C54c5ccccc5-c5ccccc54)c4ccc5c(c4)sc4cc6ccccc6cc45)cc3)cc2)cc1]

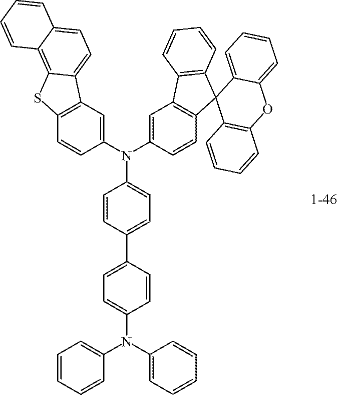 [SMILES: c1ccc(N(c2ccccc2)c2ccc(-c3ccc(N(c4ccc5c(c4)-c4ccccc4C54c5ccccc5Oc5ccccc54)c4ccc5sc6c7ccccc7ccc6c5c4)cc3)cc2)cc1]

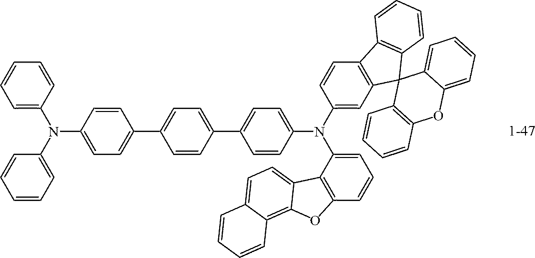 [SMILES: c1ccc(N(c2ccccc2)c2ccc(-c3ccc(-c4ccc(N(c5ccc6c(c5)C5(c7ccccc7Oc7ccccc75)c5ccccc5-6)c5cccc6oc7c8ccccc8ccc7c56)cc4)cc3)cc2)cc1]

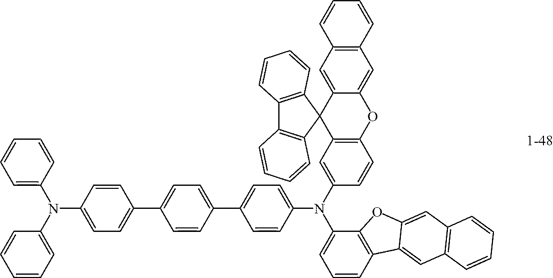 [SMILES: c1ccc(N(c2ccccc2)c2ccc(-c3ccc(-c4ccc(N(c5ccc6c(c5)C5(c7cc8ccccc8cc7O6)c6ccccc6-c6ccccc65)c5cccc6c5oc5cc7ccccc7cc56)cc4)cc3)cc2)cc1]

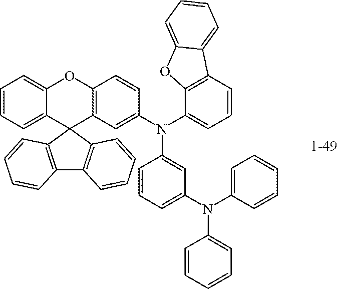 [SMILES: c1ccc(N(c2ccccc2)c2cccc(N(c3ccc4c(c3)C3(c5ccccc5O4)c4ccccc4-c4ccccc43)c3cccc4c3oc3ccccc34)c2)cc1]

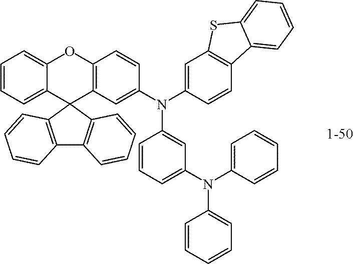 [SMILES: c1ccc(N(c2ccccc2)c2cccc(N(c3ccc4c(c3)C3(c5ccccc5O4)c4ccccc4-c4ccccc43)c3ccc4c(c3)sc3ccccc34)c2)cc1]

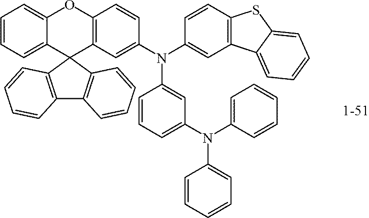 [SMILES: c1ccc(N(c2ccccc2)c2cccc(N(c3ccc4c(c3)C3(c5ccccc5O4)c4ccccc4-c4ccccc43)c3ccc4sc5ccccc5c4c3)c2)cc1]

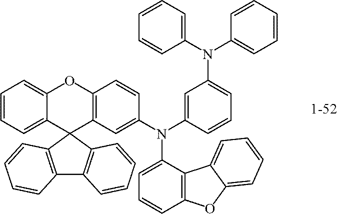 [SMILES: c1ccc(N(c2ccccc2)c2cccc(N(c3ccc4c(c3)C3(c5ccccc5O4)c4ccccc4-c4ccccc43)c3cccc4oc5ccccc5c34)c2)cc1]

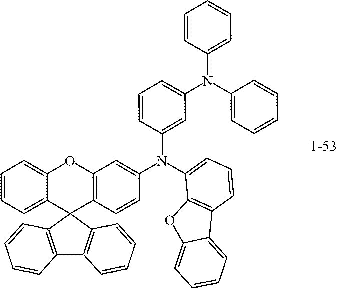 [SMILES: c1ccc(N(c2ccccc2)c2cccc(N(c3ccc4c(c3)Oc3ccccc3C43c4ccccc4-c4ccccc43)c3cccc4c3oc3ccccc34)c2)cc1]

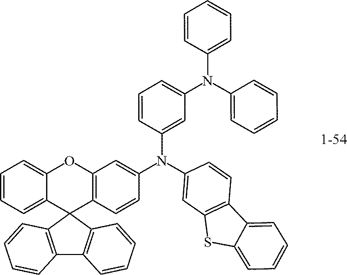 [SMILES: c1ccc(N(c2ccccc2)c2cccc(N(c3ccc4c(c3)Oc3ccccc3C43c4ccccc4-c4ccccc43)c3ccc4c(c3)sc3ccccc34)c2)cc1]

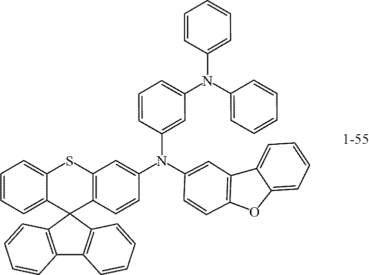 [SMILES: c1ccc(N(c2ccccc2)c2cccc(N(c3ccc4c(c3)Sc3ccccc3C43c4ccccc4-c4ccccc43)c3ccc4oc5ccccc5c4c3)c2)cc1]

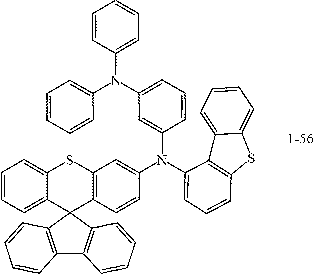 [SMILES: c1ccc(N(c2ccccc2)c2cccc(N(c3ccc4c(c3)Sc3ccccc3C43c4ccccc4-c4ccccc43)c3cccc4sc5ccccc5c34)c2)cc1]

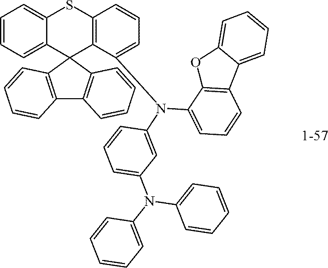 [SMILES: c1ccc(N(c2ccccc2)c2cccc(N(c3cccc4c3C3(c5ccccc5S4)c4ccccc4-c4ccccc43)c3cccc4c3oc3ccccc34)c2)cc1]

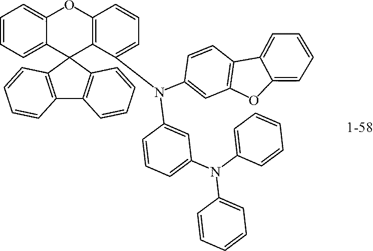 [SMILES: c1ccc(N(c2ccccc2)c2cccc(N(c3ccc4c(c3)oc3ccccc34)c3cccc4c3C3(c5ccccc5O4)c4ccccc4-c4ccccc43)c2)cc1]

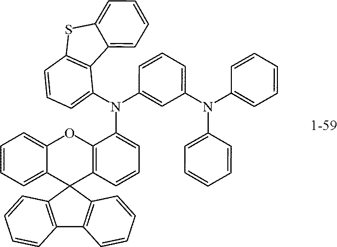 [SMILES: c1ccc(N(c2ccccc2)c2cccc(N(c3cccc4c3Oc3ccccc3C43c4ccccc4-c4ccccc43)c3cccc4sc5ccccc5c34)c2)cc1]

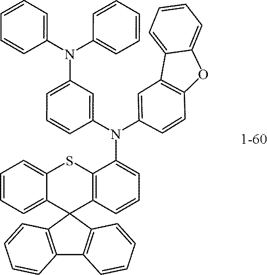 [SMILES: c1ccc(N(c2ccccc2)c2cccc(N(c3ccc4oc5ccccc5c4c3)c3cccc4c3Sc3ccccc3C43c4ccccc4-c4ccccc43)c2)cc1]

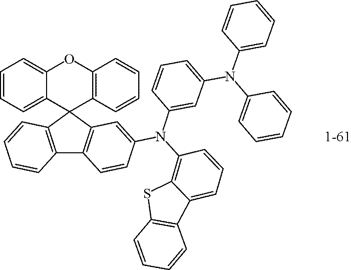 [SMILES: c1ccc(N(c2ccccc2)c2cccc(N(c3ccc4c(c3)C3(c5ccccc5Oc5ccccc53)c3ccccc3-4)c3cccc4c3sc3ccccc34)c2)cc1]

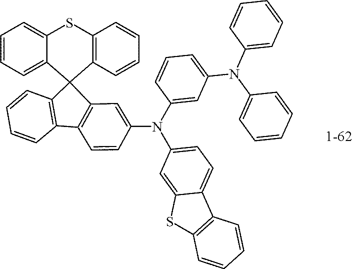 [SMILES: c1ccc(N(c2ccccc2)c2cccc(N(c3ccc4c(c3)C3(c5ccccc5Sc5ccccc53)c3ccccc3-4)c3ccc4c(c3)sc3ccccc34)c2)cc1]

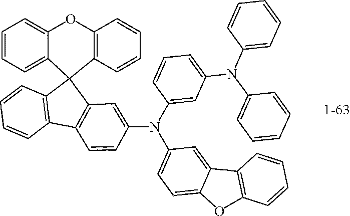 [SMILES: c1ccc(N(c2ccccc2)c2cccc(N(c3ccc4c(c3)C3(c5ccccc5Oc5ccccc53)c3ccccc3-4)c3ccc4oc5ccccc5c4c3)c2)cc1]

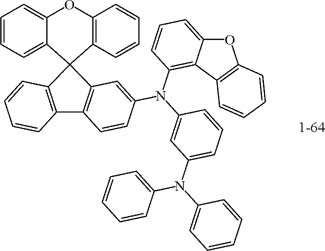 [SMILES: c1ccc(N(c2ccccc2)c2cccc(N(c3ccc4c(c3)C3(c5ccccc5Oc5ccccc53)c3ccccc3-4)c3cccc4oc5ccccc5c34)c2)cc1]

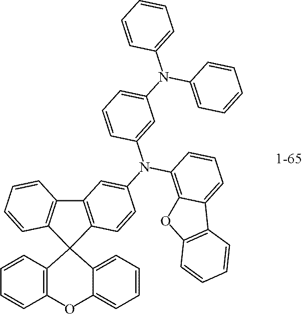 [SMILES: c1ccc(N(c2ccccc2)c2cccc(N(c3ccc4c(c3)-c3ccccc3C43c4ccccc4Oc4ccccc43)c3cccc4c3oc3ccccc34)c2)cc1]

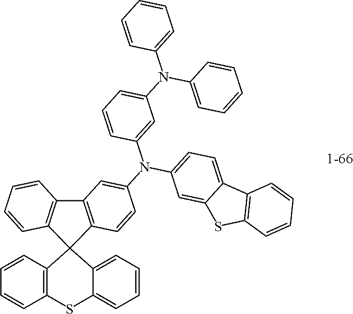 [SMILES: c1ccc(N(c2ccccc2)c2cccc(N(c3ccc4c(c3)-c3ccccc3C43c4ccccc4Sc4ccccc43)c3ccc4c(c3)sc3ccccc34)c2)cc1]

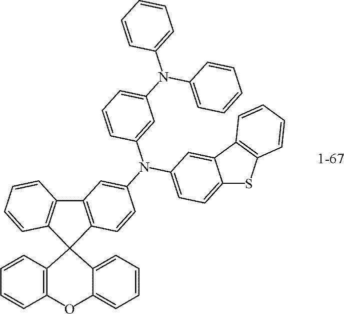 [SMILES: c1ccc(N(c2ccccc2)c2cccc(N(c3ccc4c(c3)-c3ccccc3C43c4ccccc4Oc4ccccc43)c3ccc4sc5ccccc5c4c3)c2)cc1]

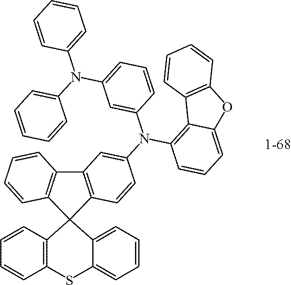 [SMILES: c1ccc(N(c2ccccc2)c2cccc(N(c3ccc4c(c3)-c3ccccc3C43c4ccccc4Sc4ccccc43)c3cccc4oc5ccccc5c34)c2)cc1]

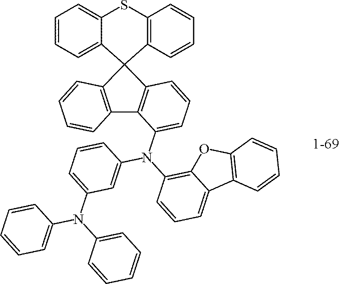 [SMILES: c1ccc(N(c2ccccc2)c2cccc(N(c3cccc4c3-c3ccccc3C43c4ccccc4Sc4ccccc43)c3cccc4c3oc3ccccc34)c2)cc1]

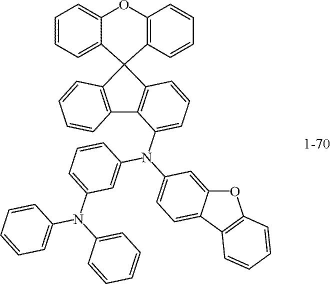 [SMILES: c1ccc(N(c2ccccc2)c2cccc(N(c3ccc4c(c3)oc3ccccc34)c3cccc4c3-c3ccccc3C43c4ccccc4Oc4ccccc43)c2)cc1]

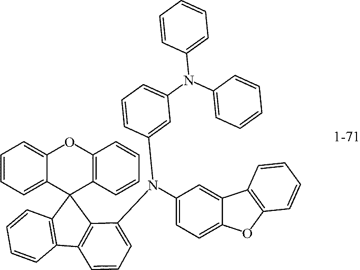 [SMILES: c1ccc(N(c2ccccc2)c2cccc(N(c3ccc4oc5ccccc5c4c3)c3cccc4c3C3(c5ccccc5Oc5ccccc53)c3ccccc3-4)c2)cc1]

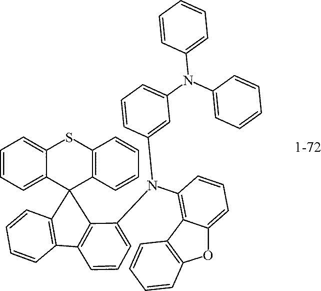 [SMILES: c1ccc(N(c2ccccc2)c2cccc(N(c3cccc4c3C3(c5ccccc5Sc5ccccc53)c3ccccc3-4)c3cccc4oc5ccccc5c34)c2)cc1]

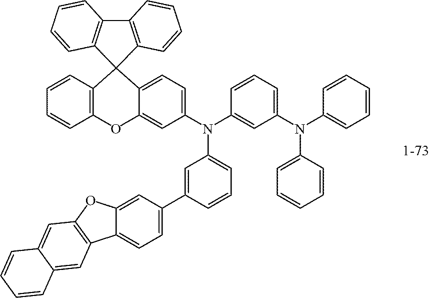 [SMILES: c1ccc(N(c2ccccc2)c2cccc(N(c3cccc(-c4ccc5c(c4)oc4cc6ccccc6cc45)c3)c3ccc4c(c3)Oc3ccccc3C43c4ccccc4-c4ccccc43)c2)cc1]

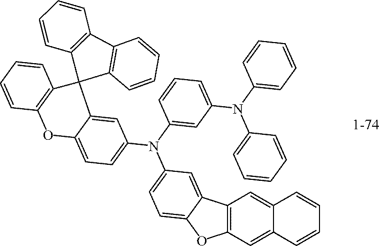 [SMILES: c1ccc(N(c2ccccc2)c2cccc(N(c3ccc4c(c3)C3(c5ccccc5O4)c4ccccc4-c4ccccc43)c3ccc4oc5cc6ccccc6cc5c4c3)c2)cc1]

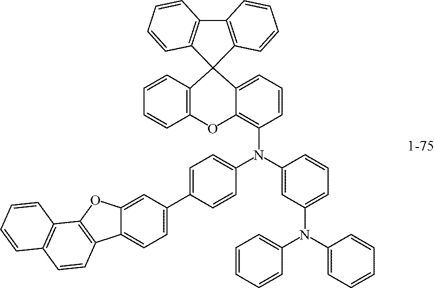 [SMILES: c1ccc(N(c2ccccc2)c2cccc(N(c3ccc(-c4ccc5c(c4)oc4c6ccccc6ccc54)cc3)c3cccc4c3Oc3ccccc3C43c4ccccc4-c4ccccc43)c2)cc1]

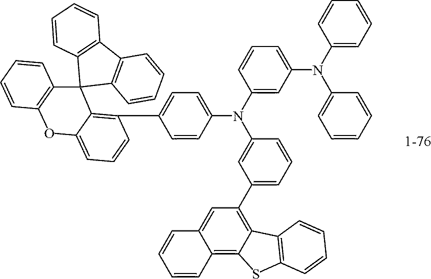 [SMILES: c1ccc(N(c2ccccc2)c2cccc(N(c3ccc(-c4cccc5c4C4(c6ccccc6O5)c5ccccc5-c5ccccc54)cc3)c3cccc(-c4cc5ccccc5c5sc6ccccc6c45)c3)c2)cc1]

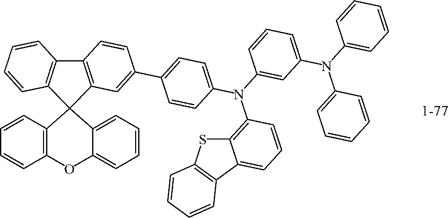 [SMILES: c1ccc(N(c2ccccc2)c2cccc(N(c3ccc(-c4ccc5c(c4)C4(c6ccccc6Oc6ccccc64)c4ccccc4-5)cc3)c3cccc4c3sc3ccccc34)c2)cc1]

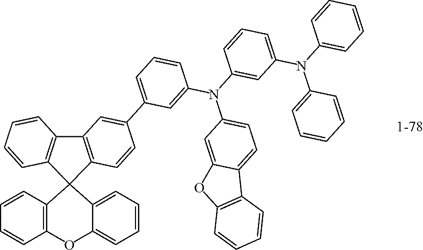 [SMILES: c1ccc(N(c2ccccc2)c2cccc(N(c3cccc(-c4ccc5c(c4)-c4ccccc4C54c5ccccc5Oc5ccccc54)c3)c3ccc4c(c3)oc3ccccc34)c2)cc1]

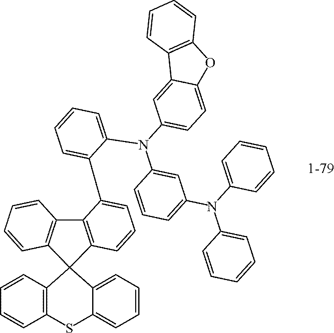 [SMILES: c1ccc(N(c2ccccc2)c2cccc(N(c3ccc4oc5ccccc5c4c3)c3ccccc3-c3cccc4c3-c3ccccc3C43c4ccccc4Sc4ccccc43)c2)cc1]

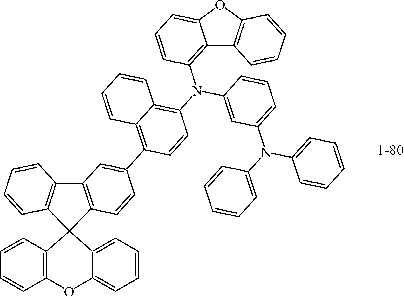 [SMILES: c1ccc(N(c2ccccc2)c2cccc(N(c3ccc(-c4ccc5c(c4)-c4ccccc4C54c5ccccc5Oc5ccccc54)c4ccccc34)c3cccc4oc5ccccc5c34)c2)cc1]

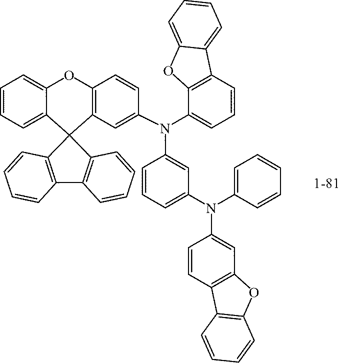 [SMILES: c1ccc(N(c2cccc(N(c3ccc4c(c3)C3(c5ccccc5O4)c4ccccc4-c4ccccc43)c3cccc4c3oc3ccccc34)c2)c2ccc3c(c2)oc2ccccc23)cc1]

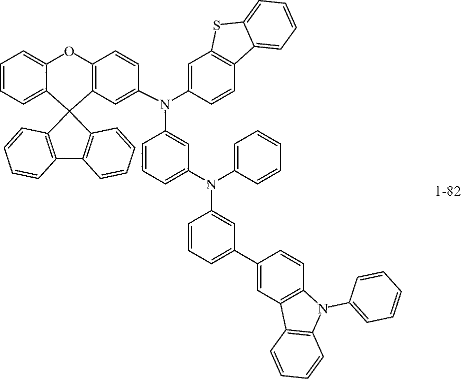 [SMILES: c1ccc(N(c2cccc(-c3ccc4c(c3)c3ccccc3n4-c3ccccc3)c2)c2cccc(N(c3ccc4c(c3)C3(c5ccccc5O4)c4ccccc4-c4ccccc43)c3ccc4c(c3)sc3ccccc34)c2)cc1]

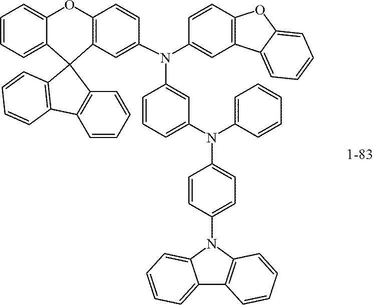 [SMILES: c1ccc(N(c2ccc(-n3c4ccccc4c4ccccc43)cc2)c2cccc(N(c3ccc4c(c3)C3(c5ccccc5O4)c4ccccc4-c4ccccc43)c3ccc4oc5ccccc5c4c3)c2)cc1]

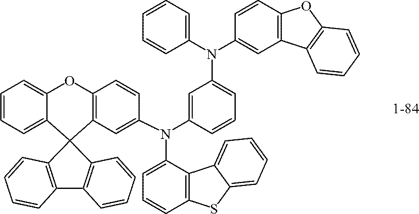 [SMILES: c1ccc(N(c2cccc(N(c3ccc4c(c3)C3(c5ccccc5O4)c4ccccc4-c4ccccc43)c3cccc4sc5ccccc5c34)c2)c2ccc3oc4ccccc4c3c2)cc1]

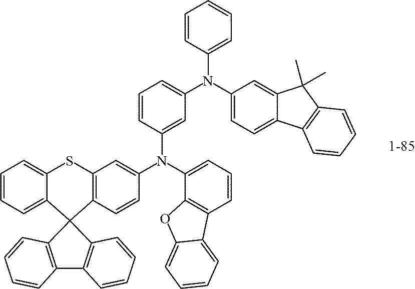 [SMILES: CC1(C)c2ccccc2-c2ccc(N(c3ccccc3)c3cccc(N(c4ccc5c(c4)Sc4ccccc4C54c5ccccc5-c5ccccc54)c4cccc5c4oc4ccccc45)c3)cc21]

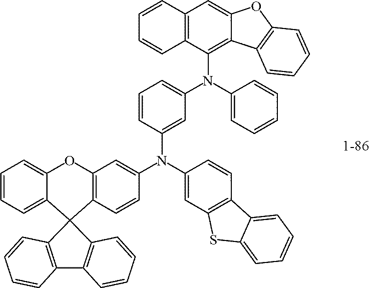 [SMILES: c1ccc(N(c2cccc(N(c3ccc4c(c3)Oc3ccccc3C43c4ccccc4-c4ccccc43)c3ccc4c(c3)sc3ccccc34)c2)c2c3ccccc3cc3oc4ccccc4c23)cc1]

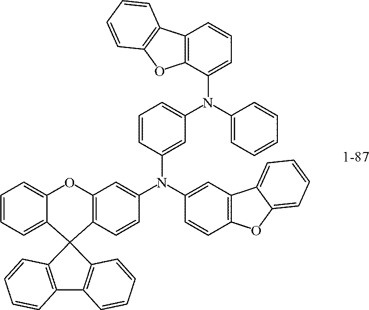 [SMILES: c1ccc(N(c2cccc(N(c3ccc4c(c3)Oc3ccccc3C43c4ccccc4-c4ccccc43)c3ccc4oc5ccccc5c4c3)c2)c2cccc3c2oc2ccccc23)cc1]

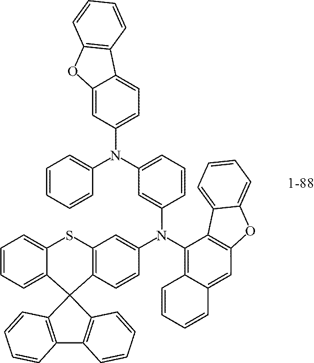 [SMILES: c1ccc(N(c2cccc(N(c3ccc4c(c3)Sc3ccccc3C43c4ccccc4-c4ccccc43)c3c4ccccc4cc4oc5ccccc5c34)c2)c2ccc3c(c2)oc2ccccc23)cc1]

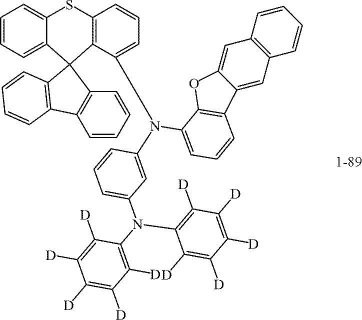 [SMILES: [2H]c1c([2H])c([2H])c(N(c2cccc(N(c3cccc4c3C3(c5ccccc5S4)c4ccccc4-c4ccccc43)c3cccc4c3oc3cc5ccccc5cc34)c2)c2c([2H])c([2H])c([2H])c([2H])c2[2H])c([2H])c1[2H]]

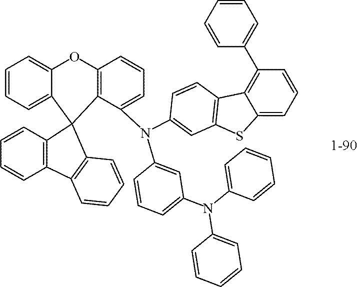 [SMILES: c1ccc(-c2cccc3sc4cc(N(c5cccc(N(c6ccccc6)c6ccccc6)c5)c5cccc6c5C5(c7ccccc7O6)c6ccccc6-c6ccccc65)ccc4c23)cc1]

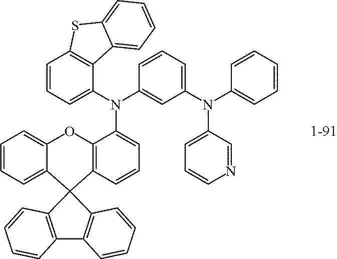 [SMILES: c1ccc(N(c2cccnc2)c2cccc(N(c3cccc4c3Oc3ccccc3C43c4ccccc4-c4ccccc43)c3cccc4sc5ccccc5c34)c2)cc1]

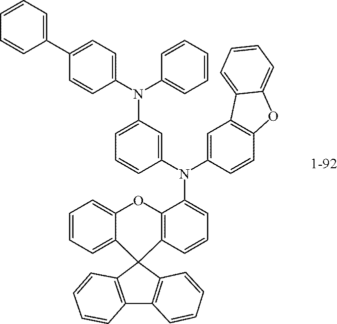 [SMILES: c1ccc(-c2ccc(N(c3ccccc3)c3cccc(N(c4ccc5oc6ccccc6c5c4)c4cccc5c4Oc4ccccc4C54c5ccccc5-c5ccccc54)c3)cc2)cc1]

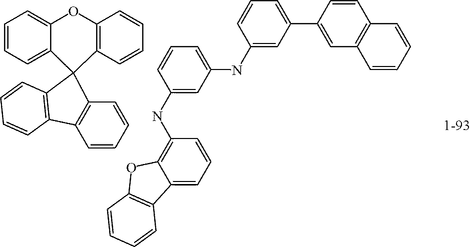

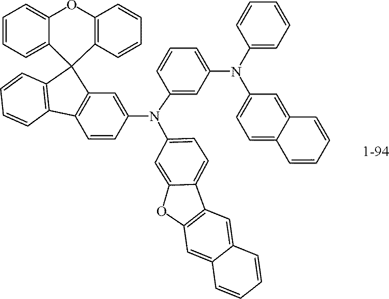 [SMILES: c1ccc(N(c2cccc(N(c3ccc4c(c3)C3(c5ccccc5Oc5ccccc53)c3ccccc3-4)c3ccc4c(c3)oc3cc5ccccc5cc34)c2)c2ccc3ccccc3c2)cc1]

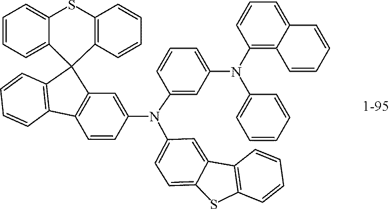 [SMILES: c1ccc(N(c2cccc(N(c3ccc4c(c3)C3(c5ccccc5Sc5ccccc53)c3ccccc3-4)c3ccc4sc5ccccc5c4c3)c2)c2cccc3ccccc23)cc1]

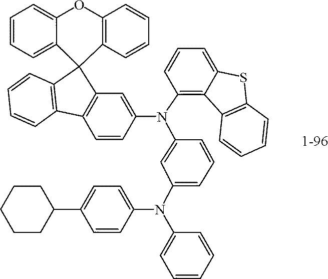 [SMILES: c1ccc(N(c2ccc(C3CCCCC3)cc2)c2cccc(N(c3ccc4c(c3)C3(c5ccccc5Oc5ccccc53)c3ccccc3-4)c3cccc4sc5ccccc5c34)c2)cc1]

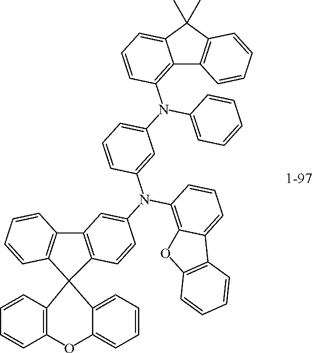 [SMILES: CC1(C)c2ccccc2-c2c(N(c3ccccc3)c3cccc(N(c4ccc5c(c4)-c4ccccc4C54c5ccccc5Oc5ccccc54)c4cccc5c4oc4ccccc45)c3)cccc21]

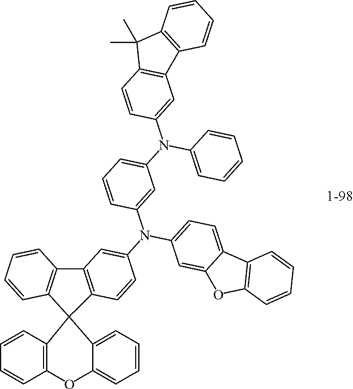 [SMILES: CC1(C)c2ccccc2-c2cc(N(c3ccccc3)c3cccc(N(c4ccc5c(c4)-c4ccccc4C54c5ccccc5Oc5ccccc54)c4ccc5c(c4)oc4ccccc45)c3)ccc21]

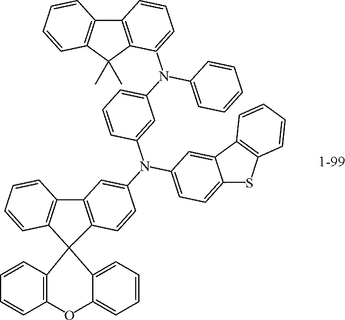

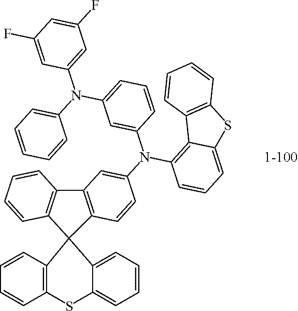 [SMILES: Fc1cc(F)cc(N(c2ccccc2)c2cccc(N(c3ccc4c(c3)-c3ccccc3C43c4ccccc4Sc4ccccc43)c3cccc4sc5ccccc5c34)c2)c1]

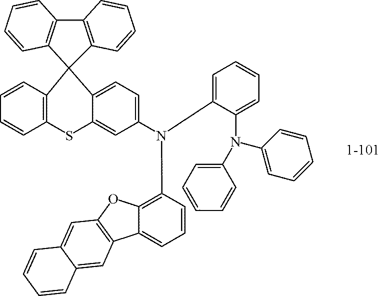 [SMILES: c1ccc(N(c2ccccc2)c2ccccc2N(c2ccc3c(c2)Sc2ccccc2C32c3ccccc3-c3ccccc32)c2cccc3c2oc2cc4ccccc4cc23)cc1]

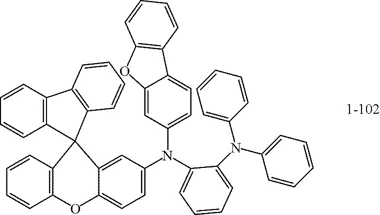 [SMILES: c1ccc(N(c2ccccc2)c2ccccc2N(c2ccc3c(c2)C2(c4ccccc4O3)c3ccccc3-c3ccccc32)c2ccc3c(c2)oc2ccccc23)cc1]

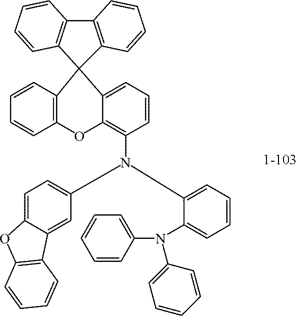 [SMILES: c1ccc(N(c2ccccc2)c2ccccc2N(c2ccc3oc4ccccc4c3c2)c2cccc3c2Oc2ccccc2C32c3ccccc3-c3ccccc32)cc1]

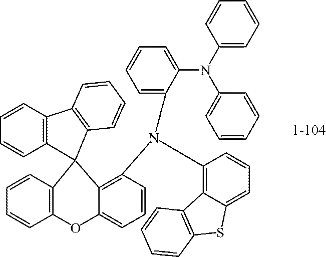 [SMILES: c1ccc(N(c2ccccc2)c2ccccc2N(c2cccc3c2C2(c4ccccc4O3)c3ccccc3-c3ccccc32)c2cccc3sc4ccccc4c23)cc1]

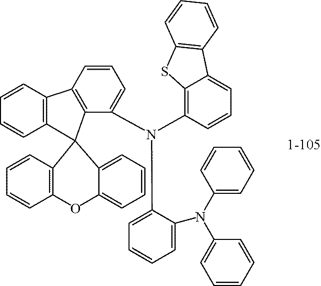 [SMILES: c1ccc(N(c2ccccc2)c2ccccc2N(c2cccc3c2C2(c4ccccc4Oc4ccccc42)c2ccccc2-3)c2cccc3c2sc2ccccc23)cc1]

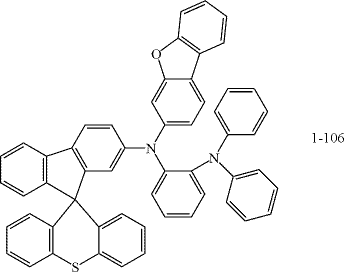 [SMILES: c1ccc(N(c2ccccc2)c2ccccc2N(c2ccc3c(c2)C2(c4ccccc4Sc4ccccc42)c2ccccc2-3)c2ccc3c(c2)oc2ccccc23)cc1]

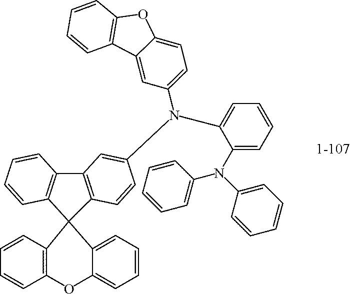 [SMILES: c1ccc(N(c2ccccc2)c2ccccc2N(c2ccc3c(c2)-c2ccccc2C32c3ccccc3Oc3ccccc32)c2ccc3oc4ccccc4c3c2)cc1]

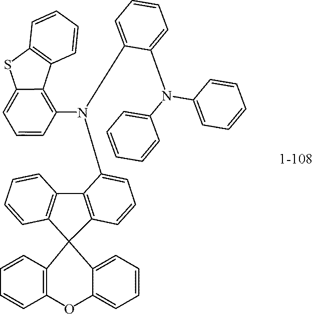 [SMILES: c1ccc(N(c2ccccc2)c2ccccc2N(c2cccc3c2-c2ccccc2C32c3ccccc3Oc3ccccc32)c2cccc3sc4ccccc4c23)cc1]

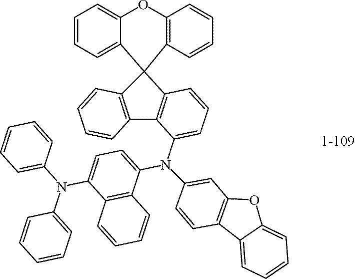 [SMILES: c1ccc(N(c2ccccc2)c2ccc(N(c3ccc4c(c3)oc3ccccc34)c3cccc4c3-c3ccccc3C43c4ccccc4Oc4ccccc43)c3ccccc23)cc1]

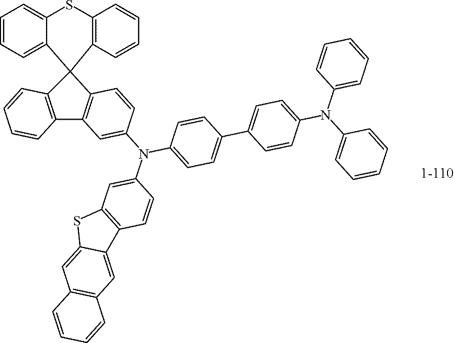 [SMILES: c1ccc(N(c2ccccc2)c2ccc(-c3ccc(N(c4ccc5c(c4)-c4ccccc4C54c5ccccc5Sc5ccccc54)c4ccc5c(c4)sc4cc6ccccc6cc45)cc3)cc2)cc1]

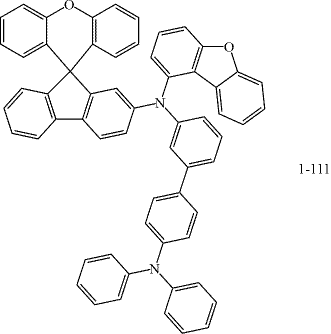 [SMILES: c1ccc(N(c2ccccc2)c2ccc(-c3cccc(N(c4ccc5c(c4)C4(c6ccccc6Oc6ccccc64)c4ccccc4-5)c4cccc5oc6ccccc6c45)c3)cc2)cc1]

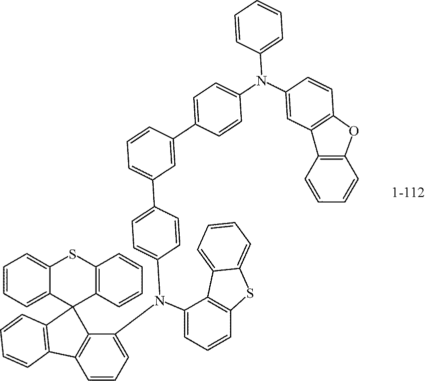 [SMILES: c1ccc(N(c2ccc(-c3cccc(-c4ccc(N(c5cccc6c5C5(c7ccccc7Sc7ccccc75)c5ccccc5-6)c5cccc6sc7ccccc7c56)cc4)c3)cc2)c2ccc3oc4ccccc4c3c2)cc1]

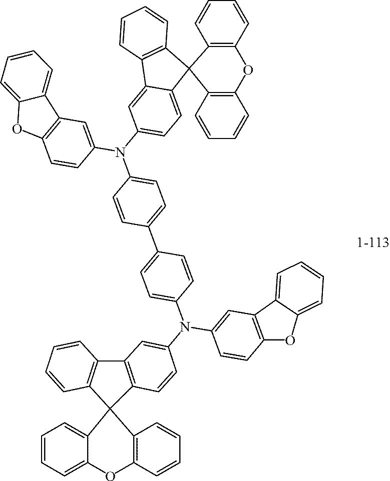 [SMILES: c1ccc2c(c1)Oc1ccccc1C21c2ccccc2-c2cc(N(c3ccc(-c4ccc(N(c5ccc6c(c5)-c5ccccc5C65c6ccccc6Oc6ccccc65)c5ccc6oc7ccccc7c6c5)cc4)cc3)c3ccc4oc5ccccc5c4c3)ccc21]

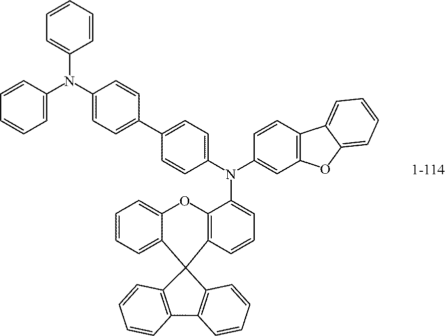 [SMILES: c1ccc(N(c2ccccc2)c2ccc(-c3ccc(N(c4ccc5c(c4)oc4ccccc45)c4cccc5c4Oc4ccccc4C54c5ccccc5-c5ccccc54)cc3)cc2)cc1]

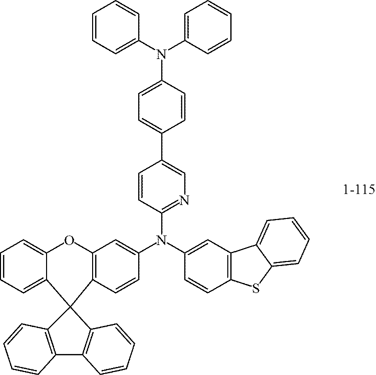 [SMILES: c1ccc(N(c2ccccc2)c2ccc(-c3ccc(N(c4ccc5c(c4)Oc4ccccc4C54c5ccccc5-c5ccccc54)c4ccc5sc6ccccc6c5c4)nc3)cc2)cc1]

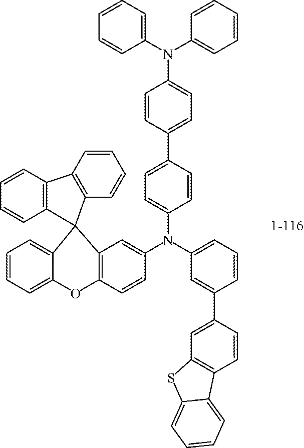 [SMILES: c1ccc(N(c2ccccc2)c2ccc(-c3ccc(N(c4cccc(-c5ccc6c(c5)sc5ccccc56)c4)c4ccc5c(c4)C4(c6ccccc6O5)c5ccccc5-c5ccccc54)cc3)cc2)cc1]

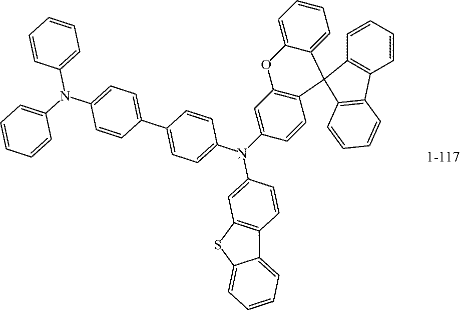 [SMILES: c1ccc(N(c2ccccc2)c2ccc(-c3ccc(N(c4ccc5c(c4)Oc4ccccc4C54c5ccccc5-c5ccccc54)c4ccc5c(c4)sc4ccccc45)cc3)cc2)cc1]

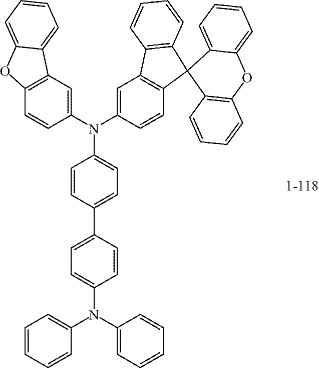 [SMILES: c1ccc(N(c2ccccc2)c2ccc(-c3ccc(N(c4ccc5c(c4)-c4ccccc4C54c5ccccc5Oc5ccccc54)c4ccc5oc6ccccc6c5c4)cc3)cc2)cc1]

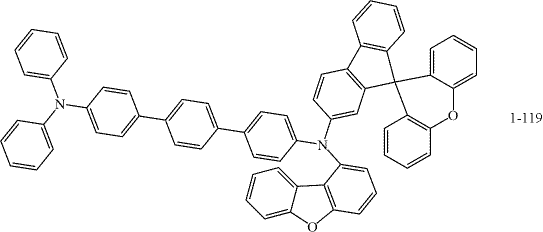 [SMILES: c1ccc(N(c2ccccc2)c2ccc(-c3ccc(-c4ccc(N(c5ccc6c(c5)C5(c7ccccc7Oc7ccccc75)c5ccccc5-6)c5cccc6oc7ccccc7c56)cc4)cc3)cc2)cc1]

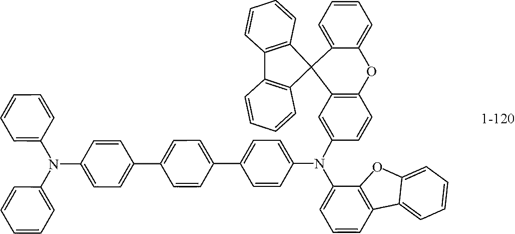 [SMILES: c1ccc(N(c2ccccc2)c2ccc(-c3ccc(-c4ccc(N(c5ccc6c(c5)C5(c7ccccc7O6)c6ccccc6-c6ccccc65)c5cccc6c5oc5ccccc56)cc4)cc3)cc2)cc1]

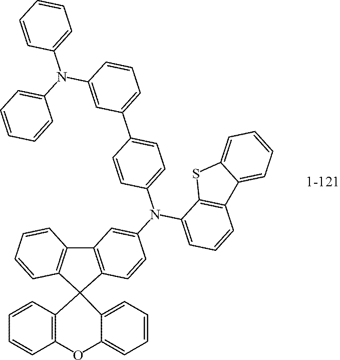 [SMILES: c1ccc(N(c2ccccc2)c2cccc(-c3ccc(N(c4ccc5c(c4)-c4ccccc4C54c5ccccc5Oc5ccccc54)c4cccc5c4sc4ccccc45)cc3)c2)cc1]

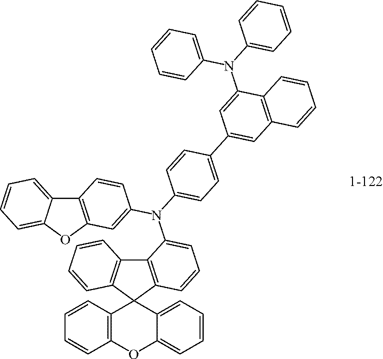 [SMILES: c1ccc(N(c2ccccc2)c2cc(-c3ccc(N(c4ccc5c(c4)oc4ccccc45)c4cccc5c4-c4ccccc4C54c5ccccc5Oc5ccccc54)cc3)cc3ccccc23)cc1]

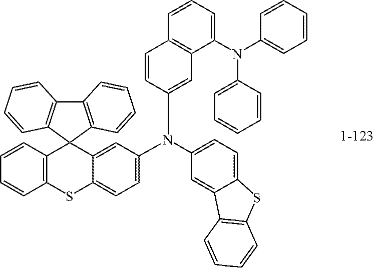 [SMILES: c1ccc(N(c2ccccc2)c2cccc3ccc(N(c4ccc5c(c4)C4(c6ccccc6S5)c5ccccc5-c5ccccc54)c4ccc5sc6ccccc6c5c4)cc23)cc1]

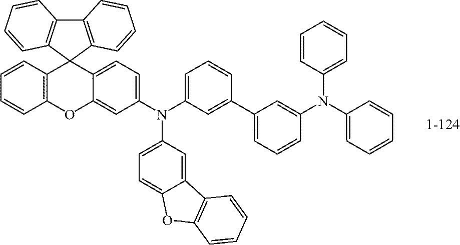 [SMILES: c1ccc(N(c2ccccc2)c2cccc(-c3cccc(N(c4ccc5c(c4)Oc4ccccc4C54c5ccccc5-c5ccccc54)c4ccc5oc6ccccc6c5c4)c3)c2)cc1]

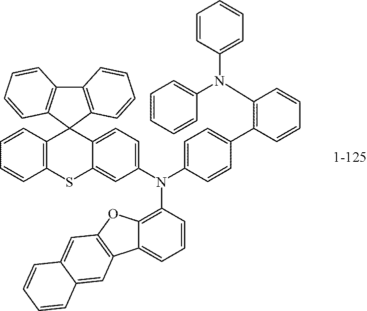 [SMILES: c1ccc(N(c2ccccc2)c2ccccc2-c2ccc(N(c3ccc4c(c3)Sc3ccccc3C43c4ccccc4-c4ccccc43)c3cccc4c3oc3cc5ccccc5cc34)cc2)cc1]

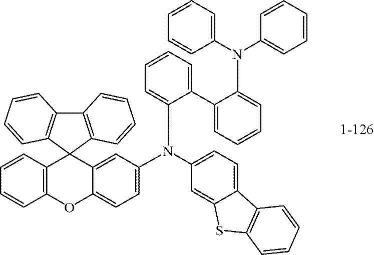 [SMILES: c1ccc(N(c2ccccc2)c2ccccc2-c2ccccc2N(c2ccc3c(c2)C2(c4ccccc4O3)c3ccccc3-c3ccccc32)c2ccc3c(c2)sc2ccccc23)cc1]

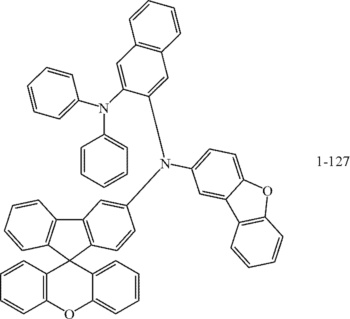 [SMILES: c1ccc(N(c2ccccc2)c2cc3ccccc3cc2N(c2ccc3c(c2)-c2ccccc2C32c3ccccc3Oc3ccccc32)c2ccc3oc4ccccc4c3c2)cc1]

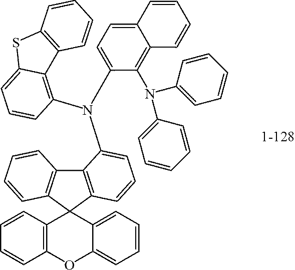 [SMILES: c1ccc(N(c2ccccc2)c2c(N(c3cccc4c3-c3ccccc3C43c4ccccc4Oc4ccccc43)c3cccc4sc5ccccc5c34)ccc3ccccc23)cc1]

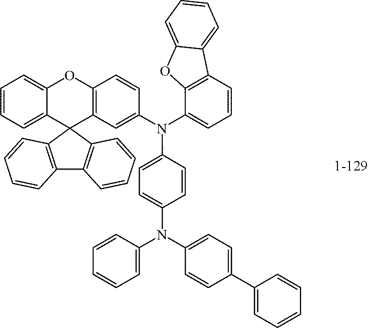 [SMILES: c1ccc(-c2ccc(N(c3ccccc3)c3ccc(N(c4ccc5c(c4)C4(c6ccccc6O5)c5ccccc5-c5ccccc54)c4cccc5c4oc4ccccc45)cc3)cc2)cc1]

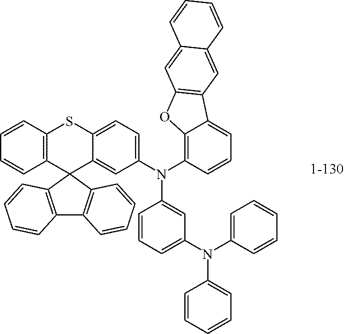 [SMILES: c1ccc(N(c2ccccc2)c2cccc(N(c3ccc4c(c3)C3(c5ccccc5S4)c4ccccc4-c4ccccc43)c3cccc4c3oc3cc5ccccc5cc34)c2)cc1]

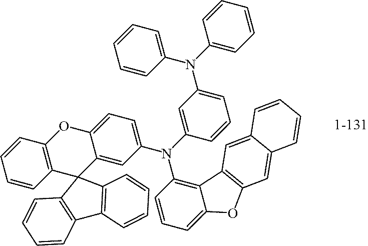 [SMILES: c1ccc(N(c2ccccc2)c2cccc(N(c3ccc4c(c3)C3(c5ccccc5O4)c4ccccc4-c4ccccc43)c3cccc4oc5cc6ccccc6cc5c34)c2)cc1]

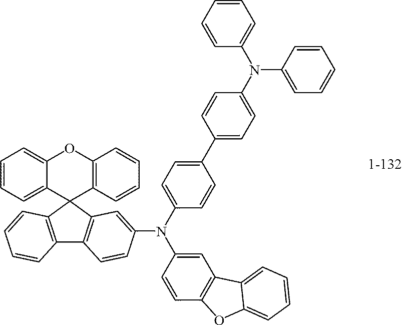 [SMILES: c1ccc(N(c2ccccc2)c2ccc(-c3ccc(N(c4ccc5c(c4)C4(c6ccccc6Oc6ccccc64)c4ccccc4-5)c4ccc5oc6ccccc6c5c4)cc3)cc2)cc1]

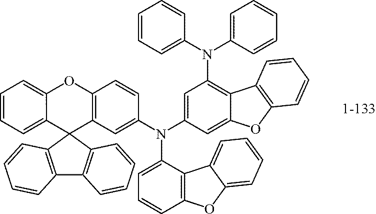 [SMILES: c1ccc(N(c2ccccc2)c2cc(N(c3ccc4c(c3)C3(c5ccccc5O4)c4ccccc4-c4ccccc43)c3cccc4oc5ccccc5c34)cc3oc4ccccc4c23)cc1]

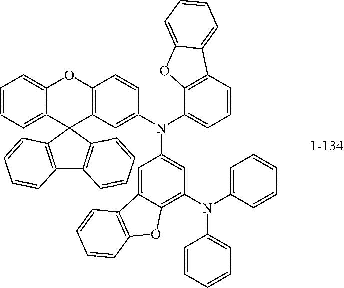 [SMILES: c1ccc(N(c2ccccc2)c2cc(N(c3ccc4c(c3)C3(c5ccccc5O4)c4ccccc4-c4ccccc43)c3cccc4c3oc3ccccc34)cc3c2oc2ccccc23)cc1]

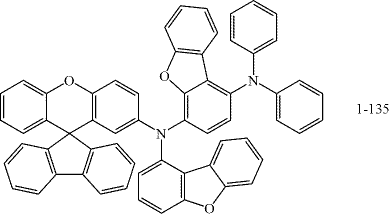 [SMILES: c1ccc(N(c2ccccc2)c2ccc(N(c3ccc4c(c3)C3(c5ccccc5O4)c4ccccc4-c4ccccc43)c3cccc4oc5ccccc5c34)c3oc4ccccc4c23)cc1]

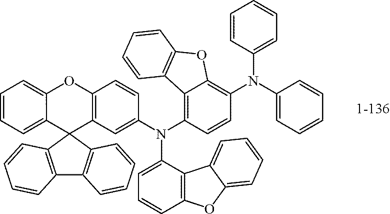 [SMILES: c1ccc(N(c2ccccc2)c2ccc(N(c3ccc4c(c3)C3(c5ccccc5O4)c4ccccc4-c4ccccc43)c3cccc4oc5ccccc5c34)c3c2oc2ccccc23)cc1]

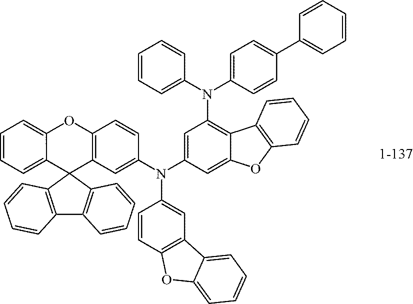 [SMILES: c1ccc(-c2ccc(N(c3ccccc3)c3cc(N(c4ccc5c(c4)C4(c6ccccc6O5)c5ccccc5-c5ccccc54)c4ccc5oc6ccccc6c5c4)cc4oc5ccccc5c34)cc2)cc1]

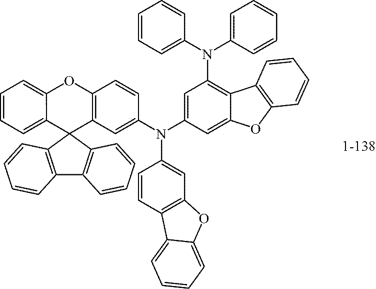 [SMILES: c1ccc(N(c2ccccc2)c2cc(N(c3ccc4c(c3)C3(c5ccccc5O4)c4ccccc4-c4ccccc43)c3ccc4c(c3)oc3ccccc34)cc3oc4ccccc4c23)cc1]

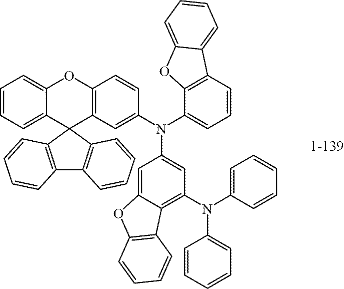 [SMILES: c1ccc(N(c2ccccc2)c2cc(N(c3ccc4c(c3)C3(c5ccccc5O4)c4ccccc4-c4ccccc43)c3cccc4c3oc3ccccc34)cc3oc4ccccc4c23)cc1]

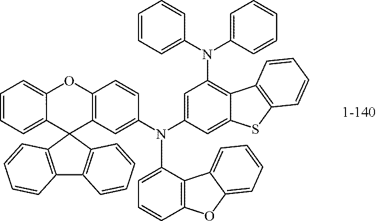 [SMILES: c1ccc(N(c2ccccc2)c2cc(N(c3ccc4c(c3)C3(c5ccccc5O4)c4ccccc4-c4ccccc43)c3cccc4oc5ccccc5c34)cc3sc4ccccc4c23)cc1]

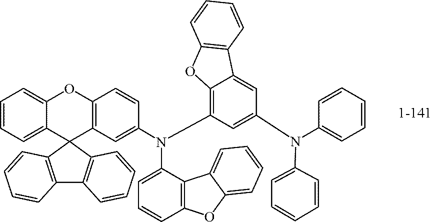 [SMILES: c1ccc(N(c2ccccc2)c2cc(N(c3ccc4c(c3)C3(c5ccccc5O4)c4ccccc4-c4ccccc43)c3cccc4oc5ccccc5c34)c3oc4ccccc4c3c2)cc1]

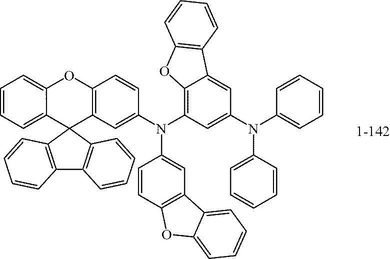 [SMILES: c1ccc(N(c2ccccc2)c2cc(N(c3ccc4c(c3)C3(c5ccccc5O4)c4ccccc4-c4ccccc43)c3ccc4oc5ccccc5c4c3)c3oc4ccccc4c3c2)cc1]

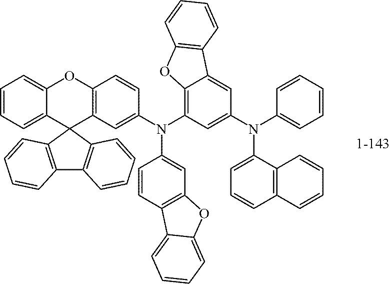 [SMILES: c1ccc(N(c2cc(N(c3ccc4c(c3)C3(c5ccccc5O4)c4ccccc4-c4ccccc43)c3ccc4c(c3)oc3ccccc34)c3oc4ccccc4c3c2)c2cccc3ccccc23)cc1]

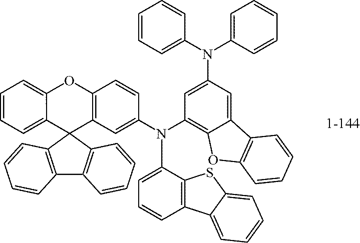 [SMILES: c1ccc(N(c2ccccc2)c2cc(N(c3ccc4c(c3)C3(c5ccccc5O4)c4ccccc4-c4ccccc43)c3cccc4c3sc3ccccc34)c3oc4ccccc4c3c2)cc1]

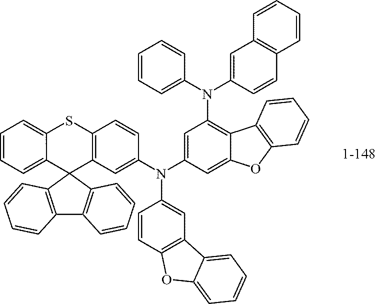 [SMILES: c1ccc(N(c2ccc3ccccc3c2)c2cc(N(c3ccc4c(c3)C3(c5ccccc5S4)c4ccccc4-c4ccccc43)c3ccc4oc5ccccc5c4c3)cc3oc4ccccc4c23)cc1]

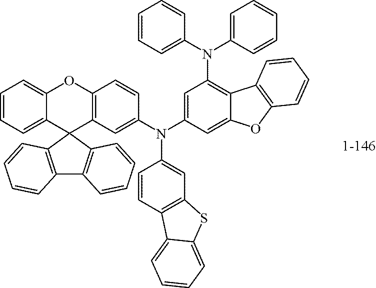 [SMILES: c1ccc(N(c2ccccc2)c2cc(N(c3ccc4c(c3)C3(c5ccccc5O4)c4ccccc4-c4ccccc43)c3ccc4c(c3)sc3ccccc34)cc3oc4ccccc4c23)cc1]

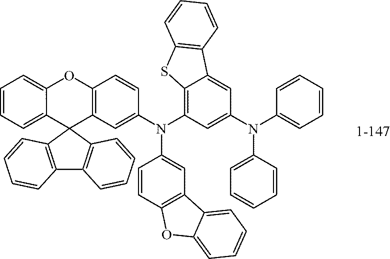 [SMILES: c1ccc(N(c2ccccc2)c2cc(N(c3ccc4c(c3)C3(c5ccccc5O4)c4ccccc4-c4ccccc43)c3ccc4oc5ccccc5c4c3)c3sc4ccccc4c3c2)cc1]

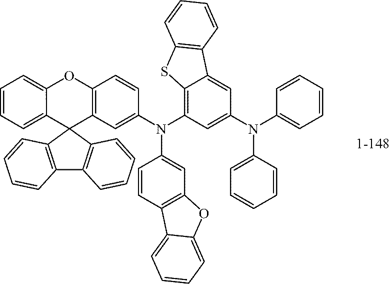 [SMILES: c1ccc(N(c2ccccc2)c2cc(N(c3ccc4c(c3)C3(c5ccccc5O4)c4ccccc4-c4ccccc43)c3ccc4c(c3)oc3ccccc34)c3sc4ccccc4c3c2)cc1]

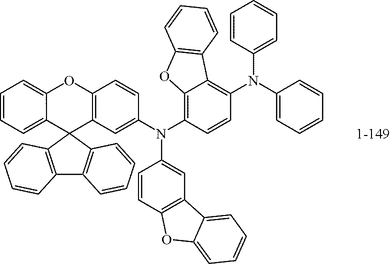 [SMILES: c1ccc(N(c2ccccc2)c2ccc(N(c3ccc4c(c3)C3(c5ccccc5O4)c4ccccc4-c4ccccc43)c3ccc4oc5ccccc5c4c3)c3oc4ccccc4c23)cc1]

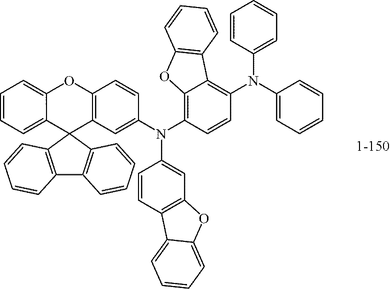 [SMILES: c1ccc(N(c2ccccc2)c2ccc(N(c3ccc4c(c3)C3(c5ccccc5O4)c4ccccc4-c4ccccc43)c3ccc4c(c3)oc3ccccc34)c3oc4ccccc4c23)cc1]

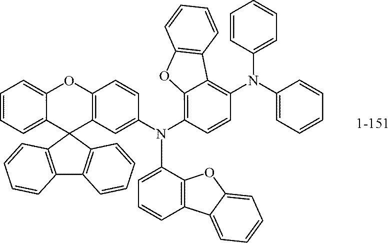 [SMILES: c1ccc(N(c2ccccc2)c2ccc(N(c3ccc4c(c3)C3(c5ccccc5O4)c4ccccc4-c4ccccc43)c3cccc4c3oc3ccccc34)c3oc4ccccc4c23)cc1]

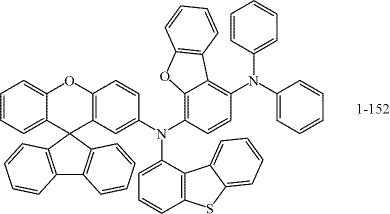 [SMILES: c1ccc(N(c2ccccc2)c2ccc(N(c3ccc4c(c3)C3(c5ccccc5O4)c4ccccc4-c4ccccc43)c3cccc4sc5ccccc5c34)c3oc4ccccc4c23)cc1]

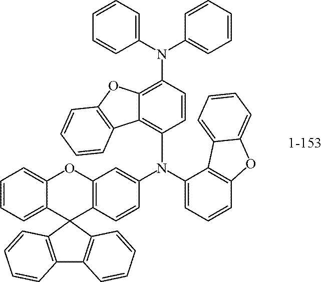 [SMILES: c1ccc(N(c2ccccc2)c2ccc(N(c3ccc4c(c3)Oc3ccccc3C43c4ccccc4-c4ccccc43)c3cccc4oc5ccccc5c34)c3c2oc2ccccc23)cc1]

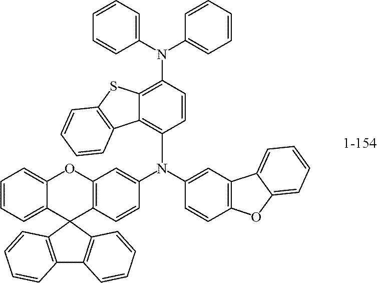 [SMILES: c1ccc(N(c2ccccc2)c2ccc(N(c3ccc4c(c3)Oc3ccccc3C43c4ccccc4-c4ccccc43)c3ccc4oc5ccccc5c4c3)c3c2sc2ccccc23)cc1]

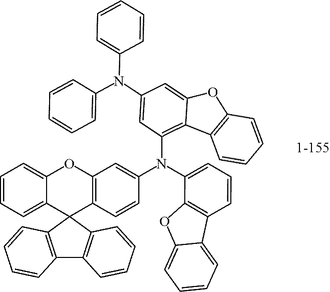 [SMILES: c1ccc(N(c2ccccc2)c2cc(N(c3ccc4c(c3)Oc3ccccc3C43c4ccccc4-c4ccccc43)c3cccc4c3oc3ccccc34)c3c(c2)oc2ccccc23)cc1]

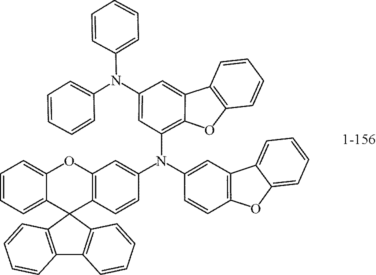 [SMILES: c1ccc(N(c2ccccc2)c2cc(N(c3ccc4c(c3)Oc3ccccc3C43c4ccccc4-c4ccccc43)c3ccc4oc5ccccc5c4c3)c3oc4ccccc4c3c2)cc1]

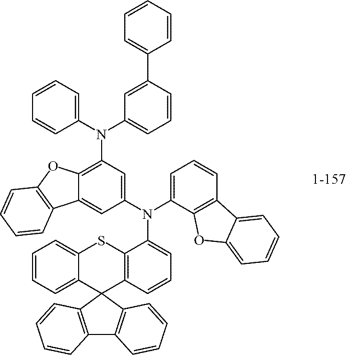 [SMILES: c1ccc(-c2cccc(N(c3ccccc3)c3cc(N(c4cccc5c4Sc4ccccc4C54c5ccccc5-c5ccccc54)c4cccc5c4oc4ccccc45)cc4c3oc3ccccc34)c2)cc1]

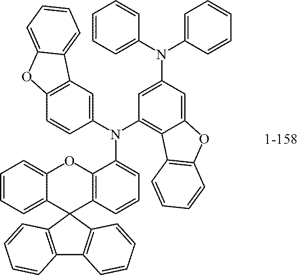 [SMILES: c1ccc(N(c2ccccc2)c2cc(N(c3ccc4oc5ccccc5c4c3)c3cccc4c3Oc3ccccc3C43c4ccccc4-c4ccccc43)c3c(c2)oc2ccccc23)cc1]

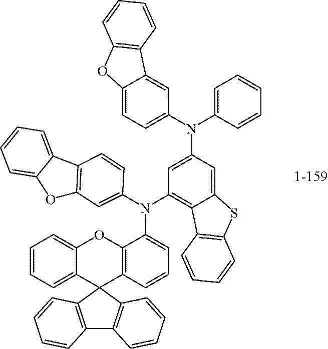 [SMILES: c1ccc(N(c2cc(N(c3ccc4c(c3)oc3ccccc34)c3cccc4c3Oc3ccccc3C43c4ccccc4-c4ccccc43)c3c(c2)sc2ccccc23)c2ccc3oc4ccccc4c3c2)cc1]

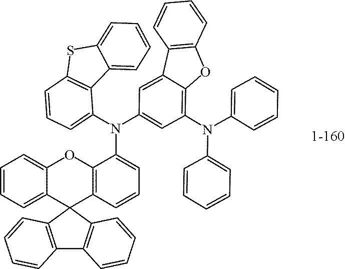 [SMILES: c1ccc(N(c2ccccc2)c2cc(N(c3cccc4c3Oc3ccccc3C43c4ccccc4-c4ccccc43)c3cccc4sc5ccccc5c34)cc3c2oc2ccccc23)cc1]

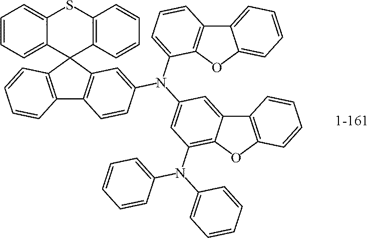 [SMILES: c1ccc(N(c2ccccc2)c2cc(N(c3ccc4c(c3)C3(c5ccccc5Sc5ccccc53)c3ccccc3-4)c3cccc4c3oc3ccccc34)cc3c2oc2ccccc23)cc1]

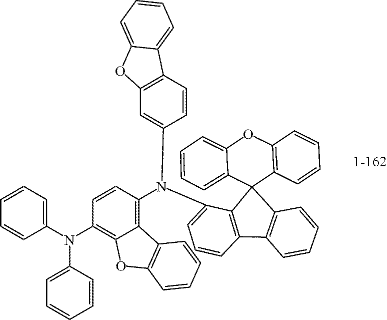 [SMILES: c1ccc(N(c2ccccc2)c2ccc(N(c3ccc4c(c3)oc3ccccc34)c3cccc4c3C3(c5ccccc5Oc5ccccc53)c3ccccc3-4)c3c2oc2ccccc23)cc1]

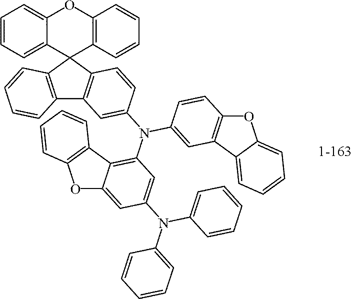 [SMILES: c1ccc(N(c2ccccc2)c2cc(N(c3ccc4c(c3)-c3ccccc3C43c4ccccc4Oc4ccccc43)c3ccc4oc5ccccc5c4c3)c3c(c2)oc2ccccc23)cc1]

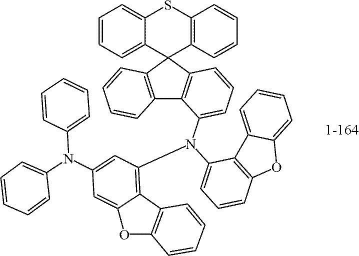 [SMILES: c1ccc(N(c2ccccc2)c2cc(N(c3cccc4c3-c3ccccc3C43c4ccccc4Sc4ccccc43)c3cccc4oc5ccccc5c34)c3c(c2)oc2ccccc23)cc1]

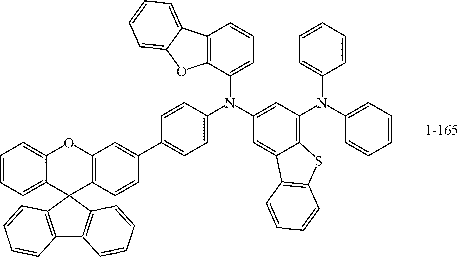 [SMILES: c1ccc(N(c2ccccc2)c2cc(N(c3ccc(-c4ccc5c(c4)Oc4ccccc4C54c5ccccc5-c5ccccc54)cc3)c3cccc4c3oc3ccccc34)cc3c2sc2ccccc23)cc1]

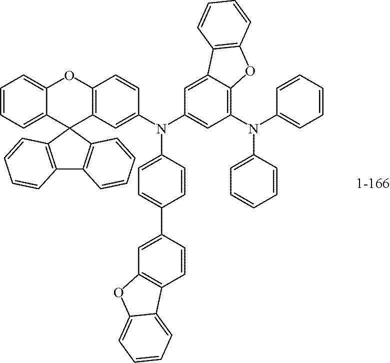 [SMILES: c1ccc(N(c2ccccc2)c2cc(N(c3ccc(-c4ccc5c(c4)oc4ccccc45)cc3)c3ccc4c(c3)C3(c5ccccc5O4)c4ccccc4-c4ccccc43)cc3c2oc2ccccc23)cc1]

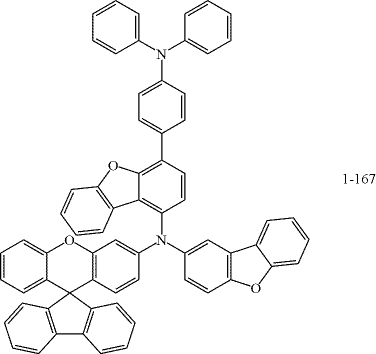 [SMILES: c1ccc(N(c2ccccc2)c2ccc(-c3ccc(N(c4ccc5c(c4)Oc4ccccc4C54c5ccccc5-c5ccccc54)c4ccc5oc6ccccc6c5c4)c4c3oc3ccccc34)cc2)cc1]

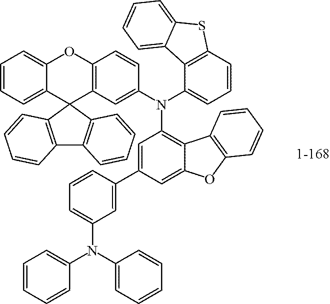 [SMILES: c1ccc(N(c2ccccc2)c2cccc(-c3cc(N(c4ccc5c(c4)C4(c6ccccc6O5)c5ccccc5-c5ccccc54)c4cccc5sc6ccccc6c45)c4c(c3)oc3ccccc34)c2)cc1]

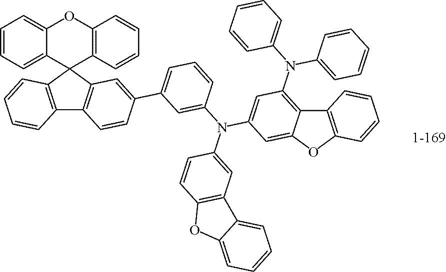 [SMILES: c1ccc(N(c2ccccc2)c2cc(N(c3cccc(-c4ccc5c(c4)C4(c6ccccc6Oc6ccccc64)c4ccccc4-5)c3)c3ccc4oc5ccccc5c4c3)cc3oc4ccccc4c23)cc1]

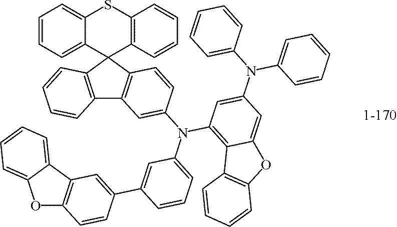 [SMILES: c1ccc(N(c2ccccc2)c2cc(N(c3cccc(-c4ccc5oc6ccccc6c5c4)c3)c3ccc4c(c3)-c3ccccc3C43c4ccccc4Sc4ccccc43)c3c(c2)oc2ccccc23)cc1]

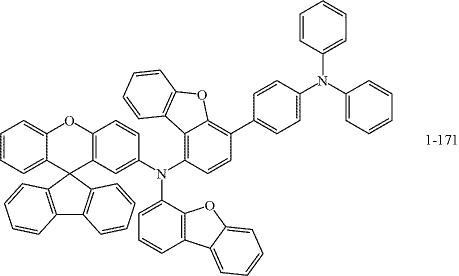 [SMILES: c1ccc(N(c2ccccc2)c2ccc(-c3ccc(N(c4ccc5c(c4)C4(c6ccccc6O5)c5ccccc5-c5ccccc54)c4cccc5c4oc4ccccc45)c4c3oc3ccccc34)cc2)cc1]

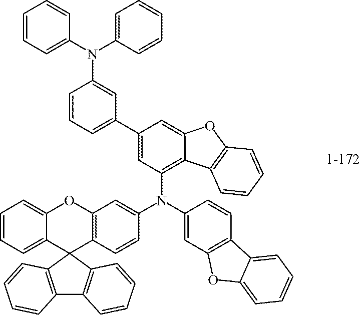 [SMILES: c1ccc(N(c2ccccc2)c2cccc(-c3cc(N(c4ccc5c(c4)Oc4ccccc4C54c5ccccc5-c5ccccc54)c4ccc5c(c4)oc4ccccc45)c4c(c3)oc3ccccc34)c2)cc1]

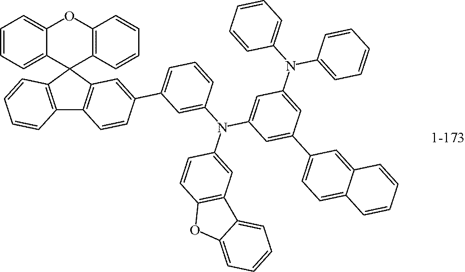 [SMILES: c1ccc(N(c2ccccc2)c2cc(-c3ccc4ccccc4c3)cc(N(c3cccc(-c4ccc5c(c4)C4(c6ccccc6Oc6ccccc64)c4ccccc4-5)c3)c3ccc4oc5ccccc5c4c3)c2)cc1]

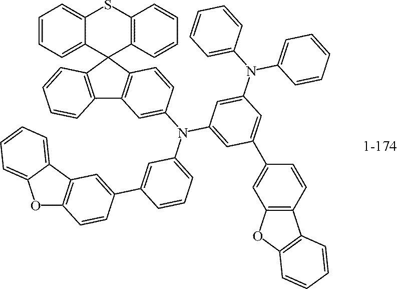 [SMILES: c1ccc(N(c2ccccc2)c2cc(-c3ccc4c(c3)oc3ccccc34)cc(N(c3cccc(-c4ccc5oc6ccccc6c5c4)c3)c3ccc4c(c3)-c3ccccc3C43c4ccccc4Sc4ccccc43)c2)cc1]

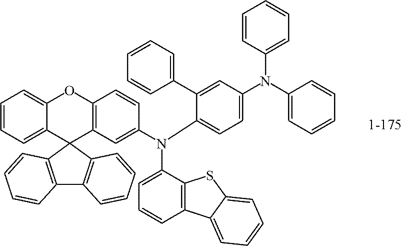 [SMILES: c1ccc(-c2cc(N(c3ccccc3)c3ccccc3)ccc2N(c2ccc3c(c2)C2(c4ccccc4O3)c3ccccc3-c3ccccc32)c2cccc3c2sc2ccccc23)cc1]

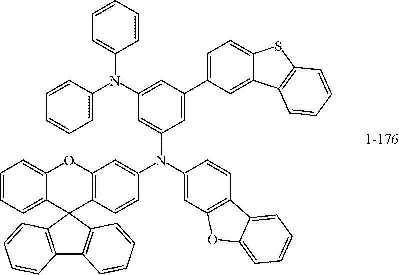 [SMILES: c1ccc(N(c2ccccc2)c2cc(-c3ccc4sc5ccccc5c4c3)cc(N(c3ccc4c(c3)Oc3ccccc3C43c4ccccc4-c4ccccc43)c3ccc4c(c3)oc3ccccc34)c2)cc1]

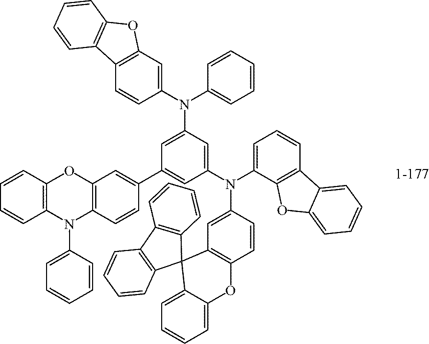 [SMILES: c1ccc(N(c2cc(-c3ccc4c(c3)Oc3ccccc3N4c3ccccc3)cc(N(c3ccc4c(c3)C3(c5ccccc5O4)c4ccccc4-c4ccccc43)c3cccc4c3oc3ccccc34)c2)c2ccc3c(c2)oc2ccccc23)cc1]

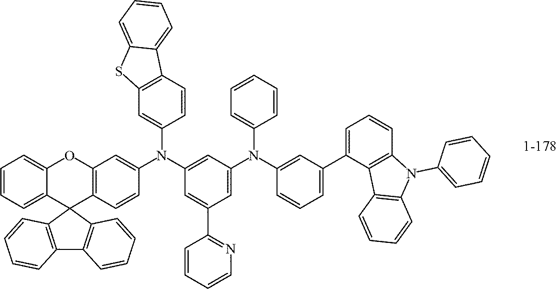 [SMILES: c1ccc(N(c2cccc(-c3cccc4c3c3ccccc3n4-c3ccccc3)c2)c2cc(-c3ccccn3)cc(N(c3ccc4c(c3)Oc3ccccc3C43c4ccccc4-c4ccccc43)c3ccc4c(c3)sc3ccccc34)c2)cc1]

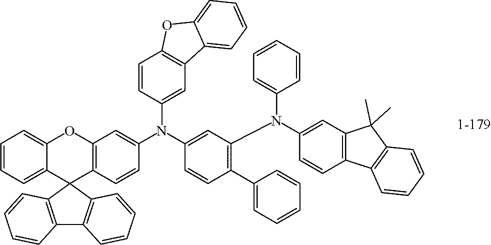 [SMILES: CC1(C)c2ccccc2-c2ccc(N(c3ccccc3)c3cc(N(c4ccc5c(c4)Oc4ccccc4C54c5ccccc5-c5ccccc54)c4ccc5oc6ccccc6c5c4)ccc3-c3ccccc3)cc21]

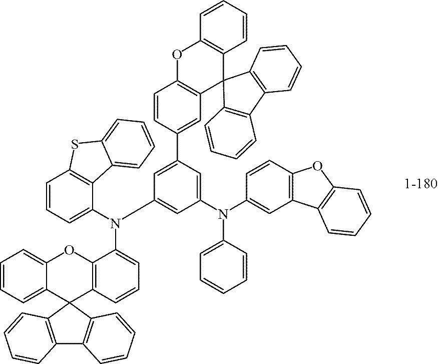 [SMILES: c1ccc(N(c2cc(-c3ccc4c(c3)C3(c5ccccc5O4)c4ccccc4-c4ccccc43)cc(N(c3cccc4c3Oc3ccccc3C43c4ccccc4-c4ccccc43)c3cccc4sc5ccccc5c34)c2)c2ccc3oc4ccccc4c3c2)cc1]

In another aspect of the present invention, the present invention provides an organic electric element including an anode, a cathode, and an organic material layer between the anode and the cathode, wherein the organic material layer includes compound represented by Formula 1.

In another aspect of the present invention, the present invention provides an organic electric element including an anode, a cathode, an organic material layer between the anode and the cathode, and a layer for improving luminous efficiency. Here, the layer for improving luminous efficiency is formed on one side of the anode or the cathode, the one side is not facing the organic material layer and the layer for improving luminous efficiency includes compound represented by Formula 1.

The organic material layer includes at least one of a hole injection layer, a hole transport layer, an emission-auxiliary layer, a light emitting layer, an electron transport-auxiliary layer, an electron transport layer and an electron injection layer, preferably, the compound may be included in the emission-auxiliary layer.

The organic material layer may include two or more stacks, wherein the stacks may include a hole transport layer, a light emitting layer, and an electron transport layer formed in sequence on the anode, and a charge generation layer may be formed between the two or more stacks.

In another aspect of the present invention, the present invention provides an electronic device including a display device and a control unit for controlling the display device, wherein the display device includes the organic electric element including compound represented by Formula 1.

Hereinafter, synthesis example of the compound represented by Formula 1 and preparation method of an organic electroluminescent element according to the present invention will be described in detail by way of examples. However, the present invention is not limited to the following examples.

### Synthesis Example

The compound represented by Formula 1 according to the present invention can be synthesized by the reaction route of the following Reaction Scheme 1, but there is no limitation thereto.

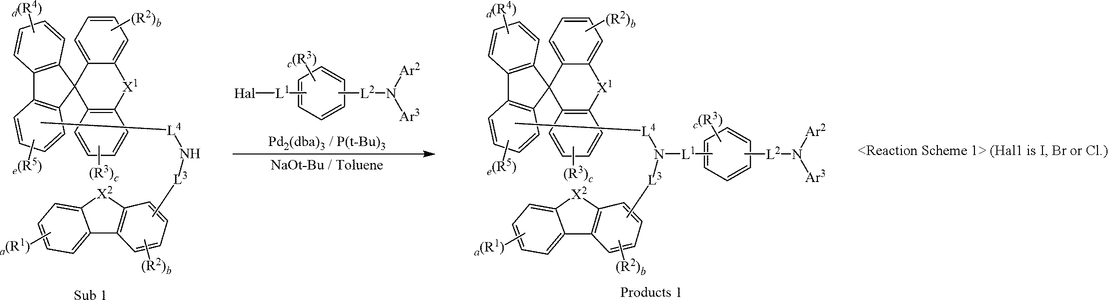

### Synthesis Example of Sub1

Sub 1 of the Reaction Scheme 1 can be synthesized according to the reaction route of the following Reaction Scheme 2, but there is no limitation thereto.

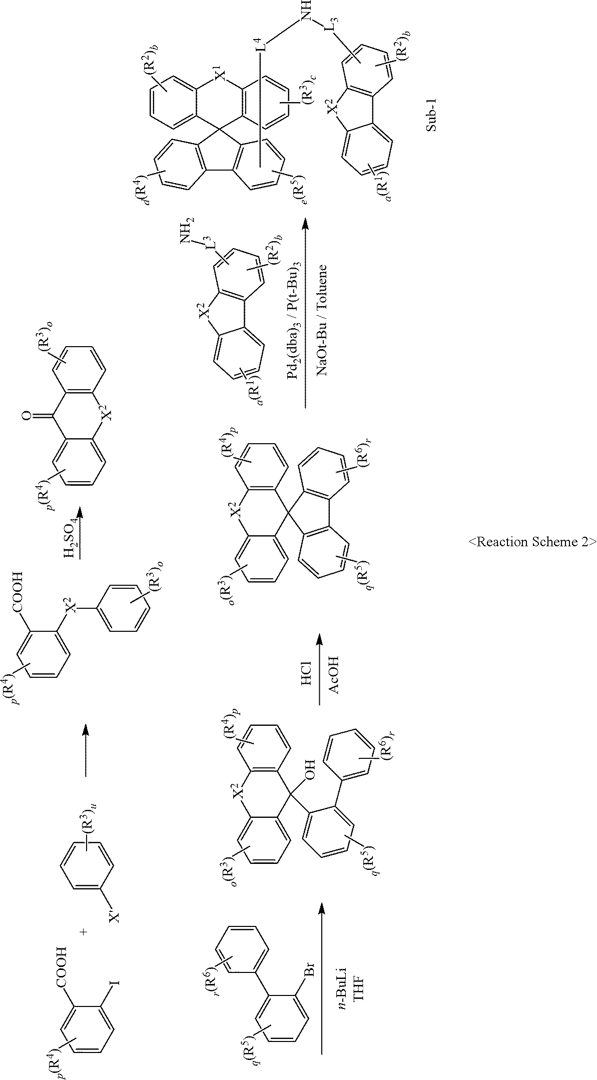

### 1. Synthesis Example of Subl-1

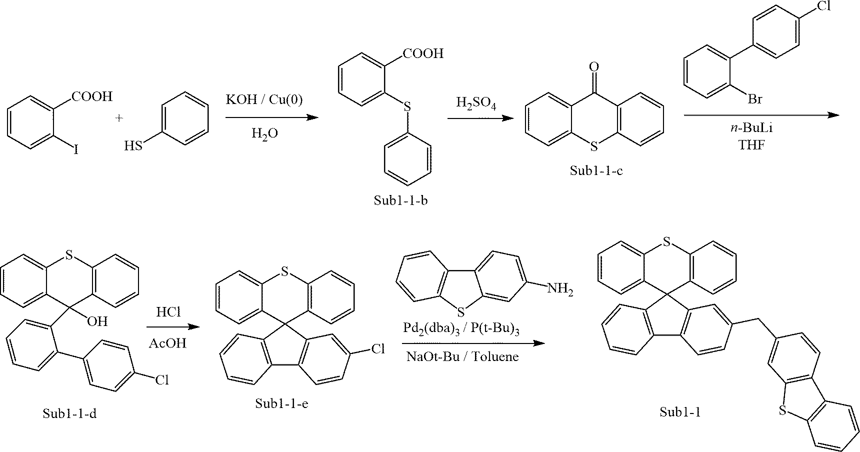

### Synthesis Example of Subl-1-B

2-iodobenzoic acid (50.0 g, 202 mmol), thiophenol (22.2 g, 202 mmol), KOH (56.6 g, 1008 mmol) and copper powder (1.3 g, 20.2 mmol) were put into a round bottom flask, water (1.3 L) was added thereto, and the mixture was refluxed for 12 hours. When the reaction was completed, the reaction product was cooled to room temperature and then 3 M HCl was added until precipitation was completed. Thereafter, the precipitate was washed with water and dried to obtain 41.3 g of product (yield: 89%).

### Synthesis Example of Subl-1-C

H₂SO₄ (1.3 mL) was added to Subl-1-b (41.3 g, 179 mmol) and the mixture was refluxed until Subl-1-b was all dissolved. When Subl-1-b was all melted, the mixture was cooled to room temperature and ice water was added to precipitate. Thereafter, the precipitate is washed with water, dried, and then dissolved with CH₂C1₂. Then, the solution was separated through a silica gel column and recrystallized to obtain 25.9 g (yield: 68%) of the product.

### Synthesis Example of Subl-1-D

After dissolving 2-bromo-4′-chloro-1,1′-biphenyl (32.6 g, 122 mmol) in THF (270 mL) under a nitrogen atmosphere, the mixture was cooled to -78° C. Then, after slowly titrating n-BuLi (49 mL) to the mixture, the titrated solution was stirred for 30 minutes, Then, after slowly titrating n-BuLi (49 mL) to the mixture, the mixture was stirred for 30 minutes and a solution of Subl-1-c (25.9 g, 122 mmol) in THF (140 mL) was slowly titrated thereto. Thereafter, the solution was stirred for 1 hour at -78° C., and the temperature is gradually raised to room temperature. When the reaction was completed, the reaction product was extracted with ethyl acetate and water, and then the organic layer was dried with MgSO₄ and concentrated. Then, the concentrate was separated through a silica gel column and recrystallized to obtain 40.1 g (yield: 82%) of the product.

### Synthesis Example of Subl-1-E

After putting Sub1-1-d (40.1 g, 100 mmol), acetic acid (250 mL) and concentrated hydrochloric acid (40 mL) in a round bottom flask, the mixture was stirred for 3 hours at 60-80° C. under a nitrogen atmosphere. When the reaction was completed, the reaction product was extracted with CH₂Cl₂ and water, and then the organic layer was dried with MgSO₄ and concentrated. Then, the concentrate was separated through a silica gel column and recrystallized to obtain 31.8 g (yield: 83%) of the product.

### Synthesis Example of Subl-1

After dissolving Subl-1-e (31.8 g, 83.0 mmol) in toluene (420 mL), dibenzo[b,d]thiophen-3-amine (16.5 g, 83.0 mmol), Pd₂(dba)₃ (2.28 g, 2.49 mmol), P(t-Bu)₃(1.01 g, 4.98 mmol) and NaOt-Bu (16.0 g, 166 mmol) were added thereto and the mixture was stirred at 90° C. When the reaction was completed, the reaction product was extracted with CH₂Cl₂ and water, and then the organic layer was dried with MgSO₄ and concentrated. Then, the concentrate was separated through a silica gel column and recrystallized to obtain 32.6 g (yield: 72%) of the product.

### 2. Synthesis Example of Sub1-29

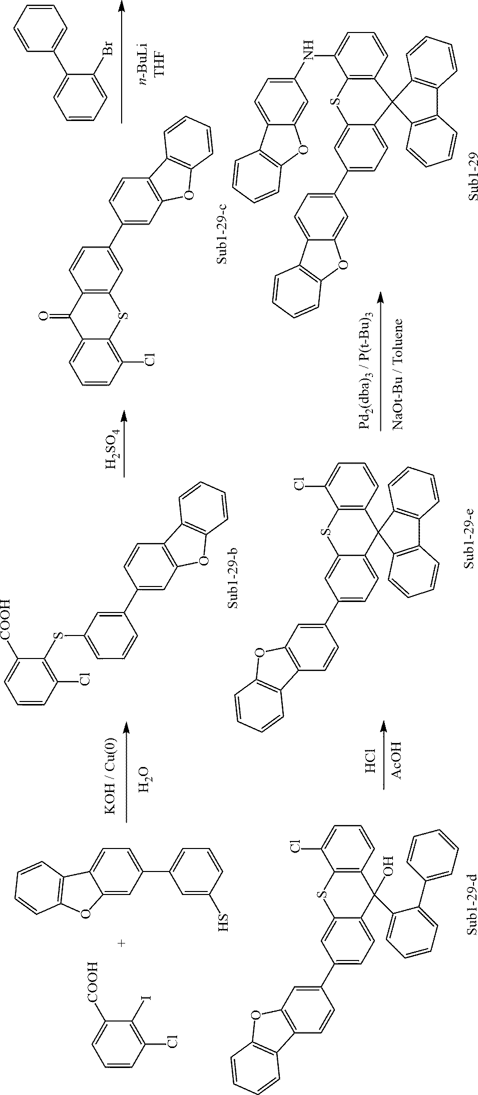

### Synthesis Example of Sub1-29-B

The reaction was carried out in the same manner as in the synthesis method of Sub1-1-b using 3-chloro-2-iodobenzoic acid (20.0 g, 70.8 mmol), 3-(dibenzo[b,d]furan-3-yl)benzenethiol (19.6 g, 70.8 mmol), KOH (19.9 g, 354 mmol) and copper powder (0.45 g, 7.08 mmol) to obtain 25.6 g (yield: 84%) of product.

### Synthesis Example of Sub1-29-C

The reaction was carried out in the same manner as in the synthesis method of Sub1-1-c using Sub1-29-b (25.6 g, 59.4 mmol) and H₂SO₄ (420 mL) to obtain 14.7 g (yield: 60%) of product.

### Synthesis Example of Sub1-29-D

The reaction was carried out in the same manner as in the synthesis method of Sub1-1-d using 2-bromo-1.1′-biphenyl (8.3 g, 35.6 mmol), n-BuLi (14 mL) and Sub1-29-c (14.7 g, 35.6 mmol) to obtain 16.4 g (yield: 81%) of product.

### Synthesis Example of Sub1-29-E

The reaction was carried out in the same manner as in the synthesis method of Sub1-1-e using Sub1-29-d (16.4 g, 28.8 mmol), acetic acid (72 mL) and concentrated hydrochloric acid (12 mL) to obtain 12.0 g (yield: 76%) of product.

### Synthesis Example of Sub1-29

The reaction was carried out in the same manner as in the synthesis method of Sub1-1 using Sub1-29-e (12.0 g, 21.9 mmol), dibenzo[b,d]furan-3-amine (4.0 g, 21.9 mmol), Pd₂(dba)₃ (0.60 g, 0.66 mmol), P(t-Bu)₃(0.27 g, 1.31 mmol) and NaOt-Bu (4.2 g, 43.7 mmol) to obtain 10.8 g (yield: 71%) of product.

### 3. Synthesis Example of Sub1-48

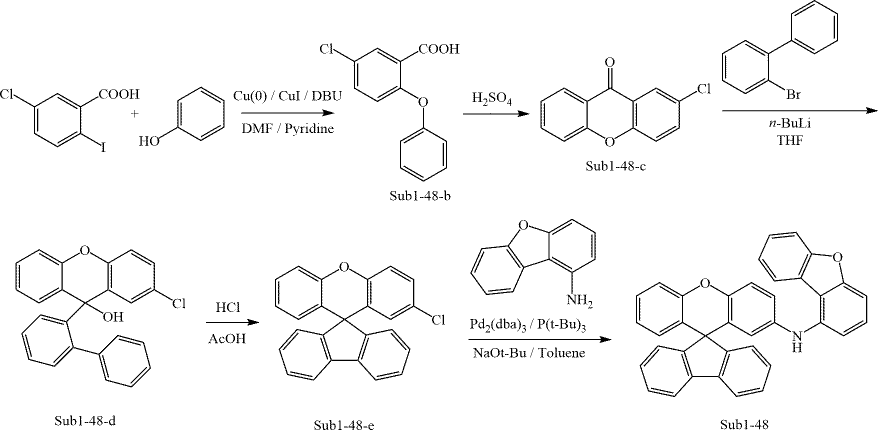

### Synthesis Example of Sub1-48-B

The reaction was carried out in the same manner as in the synthesis method of Sub1-1-b using 5-chloro-2-iodobenzoic acid (50.0 g, 177 mmol), Phenol (33.3 g, 354 mmol), 1,8-diazabicyclo[5.4.0]undec-7-ene (DBU) (79.4 g, 531 mmol), pyridine (2.9 mL), copper powder (1.5 g, 23.0 mmol) and CuI (1.5 g, 7.97 mmol) to obtain 38.3 g (yield: 87%) of product.

### Synthesis Example of Sub1-48-c

The reaction was carried out in the same manner as in the synthesis method of Sub1-1-c using Sub1-48-b (38.3 g, 154 mmol) and H₂SO₄ (1.1 L) to obtain 23.1 g (yield: 65%) of product.

### Synthesis Example of Sub1-48-D

The reaction was carried out in the same manner as in the synthesis method of Sub1-1-d using 2-bromo-1,1′-biphenyl (23.3 g, 100 mmol), n-BuLi (40 mL) and Sub1-48-c (23.1 g, 100 mmol) to obtain 32.8 g (yield: 85%) of product.

### Synthesis Example of Sub1-48-E

The reaction was carried out in the same manner as in the synthesis method of Sub1-1-e using Sub1-48-d (32.8 g, 85.1 mmol), acetic acid (210 mL) and concentrated hydrochloric acid (35 mL) to obtain 28.4 g (yield: 91%) of product.

### Synthesis Example of Sub1-48

The reaction was carried out in the same manner as in the synthesis method of Sub1-1 using Sub1-48-e (28.4 g, 77.4 mmol), dibenzo[b,d]furan-1-amine (14.2 g, 77.4 mmol), Pd₂(dba)₃ (2.13 g, 2.32 mmol), P(t-Bu)₃(0.94 g, 4.65 mmol) and NaOt-Bu (14.9 g, 155 mmol) to obtain 29.8 g (yield: 75%) of product.

### 4. Synthesis Example of Sub1-67

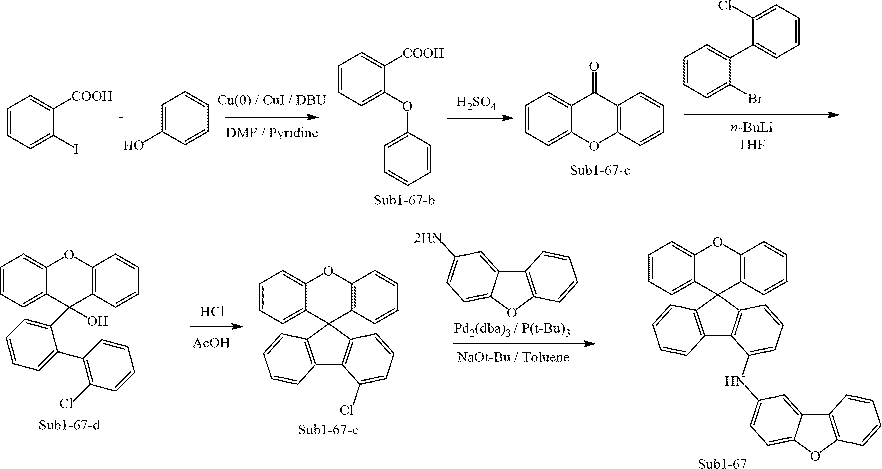

### Synthesis Example of Sub1-67-b

2-iodobenzoic acid (50.0 g, 202 mmol), Phenol (37.9 g, 403 mmol), 1,8-diazabicyclo[5.4.0]undec-7-ene (DBU) (90.4 g, 605 mmol), pyridine (3.2 mL), copper powder (1.7 g, 26.2 mmol) and CuI (1.7 g, 9.07 mmol) were put into a round bottom flask, DMF (1.6 L) was added thereto, and the mixture was refluxed for 3 hours. When the reaction was completed, the reaction product was cooled to room temperature and then 3 M HC1 was added until precipitation was completed. Thereafter, the precipitate was washed with water and dried to obtain 41.8 g (yield: 86%).

### Synthesis Example of Sub1-67-C

The reaction was carried out in the same manner as in the synthesis method of Sub1-1-c using Sub1-67-b (41.8 g, 195 mmol) and H₂SO₄ (14 L) to obtain 26.4 g (yield: 69%) of product.

### Synthesis Example of Sub1-67-D

The reaction was carried out in the same manner as in the synthesis method of Sub1-1-d using 2-bromo-2′-chloro-1,1′-biphenyl (36.0 g, 135 mmol), n-BuLi (54 mL) and Sub1-67-c (26.4 g, 135 mmol) to obtain 45.1 g (yield: 87%) of product.

### Synthesis Example of Sub1-67-E

The reaction was carried out in the same manner as in the synthesis method of Sub1-1-e using Sub1-67-d (45.1 g, 117 mmol), acetic acid (290 mL) and concentrated hydrochloric acid 50 mL) to obtain 40.4 g (yield: 94%) of product.

### Synthesis Example of Sub1-67

The reaction was carried out in the same manner as in the synthesis method of Sub1-1 using Sub1-67-e (40.4 g, 110 mmol), dibenzo[b,d]furan-2-amine (20.2 g, 110 mmol), Pd₂(dba)₃ (3.03 g, 3.30 mmol), P(t-Bu)₃(1.34 g, 6.61 mmol) and NaOt-Bu (21.2 g, 220 mmol) to obtain 38.4 g (yield: 68%) of product.

The compounds belonging to Sub 1 may be, but not limited to, the following compounds, and Table 1 shows FD-MS(Field Desorption-Mass Spectrometry) values of the following compounds.

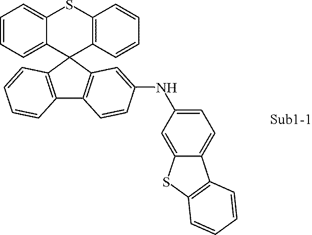 [SMILES: c1ccc2c(c1)Sc1ccccc1C21c2ccccc2-c2ccc(Nc3ccc4c(c3)sc3ccccc34)cc21]

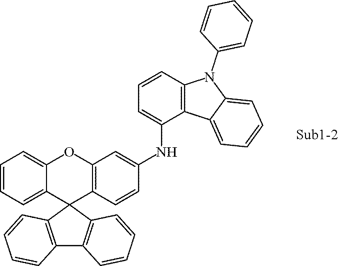 [SMILES: c1ccc(-n2c3ccccc3c3c(Nc4ccc5c(c4)Oc4ccccc4C54c5ccccc5-c5ccccc54)cccc32)cc1]

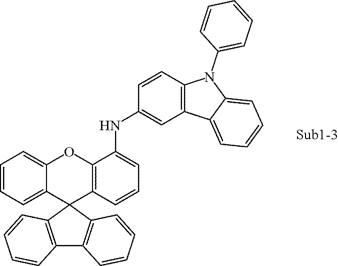 [SMILES: c1ccc(-n2c3ccccc3c3cc(Nc4cccc5c4Oc4ccccc4C54c5ccccc5-c5ccccc54)ccc32)cc1]

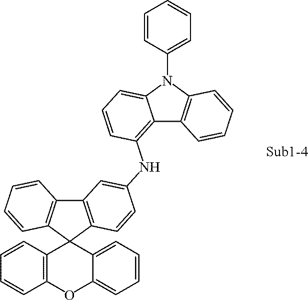 [SMILES: c1ccc(-n2c3ccccc3c3c(Nc4ccc5c(c4)-c4ccccc4C54c5ccccc5Oc5ccccc54)cccc32)cc1]

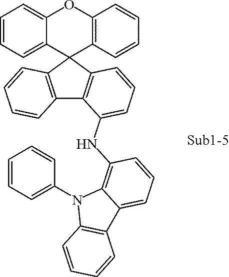 [SMILES: c1ccc(-n2c3ccccc3c3cccc(Nc4cccc5c4-c4ccccc4C54c5ccccc5Oc5ccccc54)c32)cc1]

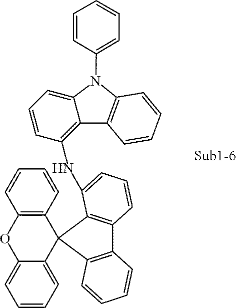 [SMILES: c1ccc(-n2c3ccccc3c3c(Nc4cccc5c4C4(c6ccccc6Oc6ccccc64)c4ccccc4-5)cccc32)cc1]

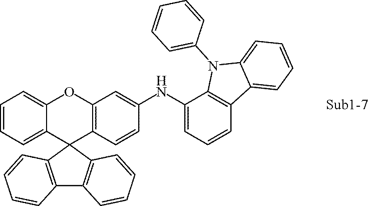 [SMILES: c1ccc(-n2c3ccccc3c3cccc(Nc4ccc5c(c4)Oc4ccccc4C54c5ccccc5-c5ccccc54)c32)cc1]

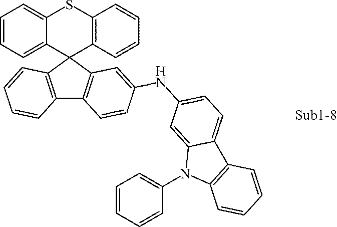 [SMILES: c1ccc(-n2c3ccccc3c3ccc(Nc4ccc5c(c4)C4(c6ccccc6Sc6ccccc64)c4ccccc4-5)cc32)cc1]

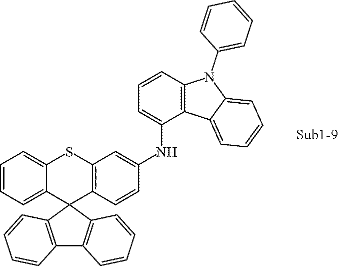 [SMILES: c1ccc(-n2c3ccccc3c3c(Nc4ccc5c(c4)Sc4ccccc4C54c5ccccc5-c5ccccc54)cccc32)cc1]

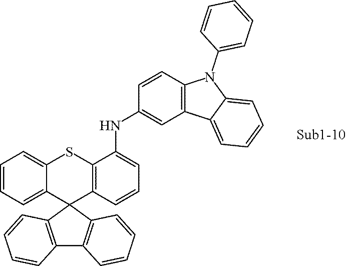 [SMILES: c1ccc(-n2c3ccccc3c3cc(Nc4cccc5c4Sc4ccccc4C54c5ccccc5-c5ccccc54)ccc32)cc1]

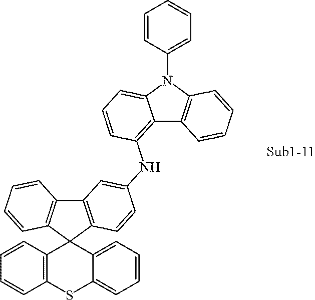 [SMILES: c1ccc(-n2c3ccccc3c3c(Nc4ccc5c(c4)-c4ccccc4C54c5ccccc5Sc5ccccc54)cccc32)cc1]

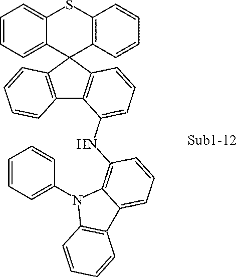 [SMILES: c1ccc(-n2c3ccccc3c3cccc(Nc4cccc5c4-c4ccccc4C54c5ccccc5Sc5ccccc54)c32)cc1]

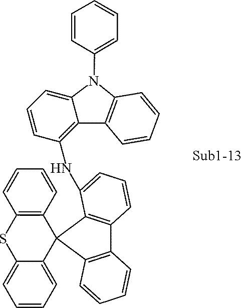 [SMILES: c1ccc(-n2c3ccccc3c3c(Nc4cccc5c4C4(c6ccccc6Sc6ccccc64)c4ccccc4-5)cccc32)cc1]

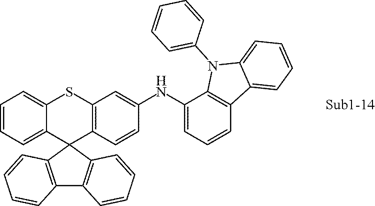 [SMILES: c1ccc(-n2c3ccccc3c3cccc(Nc4ccc5c(c4)Sc4ccccc4C54c5ccccc5-c5ccccc54)c32)cc1]

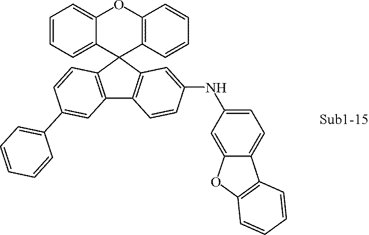 [SMILES: c1ccc(-c2ccc3c(c2)-c2ccc(Nc4ccc5c(c4)oc4ccccc45)cc2C32c3ccccc3Oc3ccccc32)cc1]

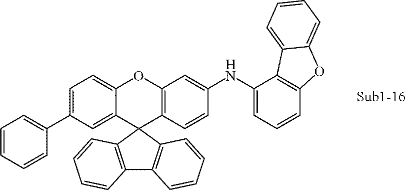 [SMILES: c1ccc(-c2ccc3c(c2)C2(c4ccc(Nc5cccc6oc7ccccc7c56)cc4O3)c3ccccc3-c3ccccc32)cc1]

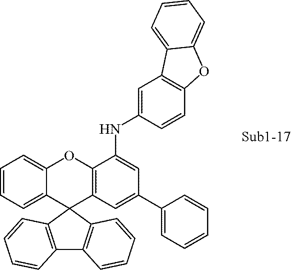 [SMILES: c1ccc(-c2cc(Nc3ccc4oc5ccccc5c4c3)c3c(c2)C2(c4ccccc4O3)c3ccccc3-c3ccccc32)cc1]

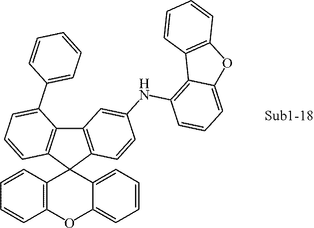 [SMILES: c1ccc(-c2cccc3c2-c2cc(Nc4cccc5oc6ccccc6c45)ccc2C32c3ccccc3Oc3ccccc32)cc1]

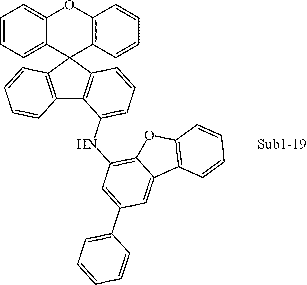 [SMILES: c1ccc(-c2cc(Nc3cccc4c3-c3ccccc3C43c4ccccc4Oc4ccccc43)c3oc4ccccc4c3c2)cc1]

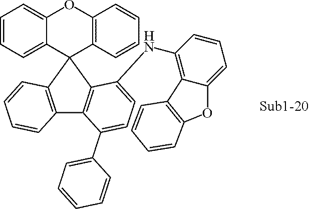 [SMILES: c1ccc(-c2ccc(Nc3cccc4oc5ccccc5c34)c3c2-c2ccccc2C32c3ccccc3Oc3ccccc32)cc1]

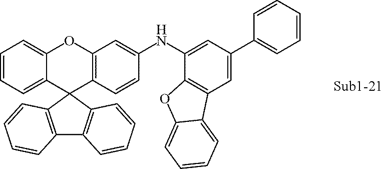 [SMILES: c1ccc(-c2cc(Nc3ccc4c(c3)Oc3ccccc3C43c4ccccc4-c4ccccc43)c3oc4ccccc4c3c2)cc1]

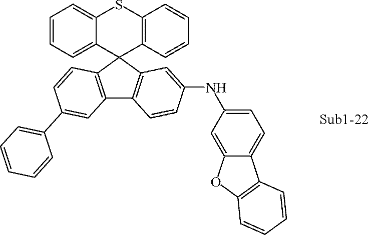 [SMILES: c1ccc(-c2ccc3c(c2)-c2ccc(Nc4ccc5c(c4)oc4ccccc45)cc2C32c3ccccc3Sc3ccccc32)cc1]

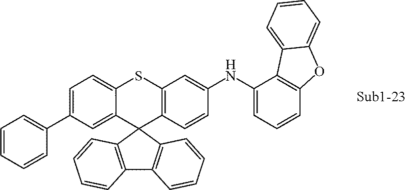 [SMILES: c1ccc(-c2ccc3c(c2)C2(c4ccc(Nc5cccc6oc7ccccc7c56)cc4S3)c3ccccc3-c3ccccc32)cc1]

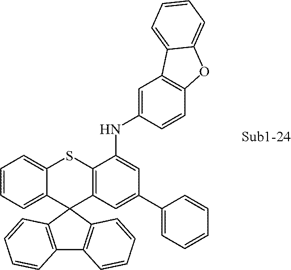 [SMILES: c1ccc(-c2cc(Nc3ccc4oc5ccccc5c4c3)c3c(c2)C2(c4ccccc4S3)c3ccccc3-c3ccccc32)cc1]

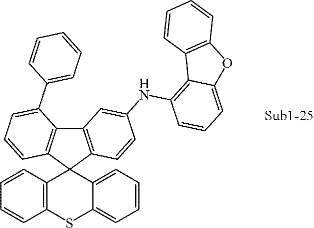 [SMILES: c1ccc(-c2cccc3c2-c2cc(Nc4cccc5oc6ccccc6c45)ccc2C32c3ccccc3Sc3ccccc32)cc1]

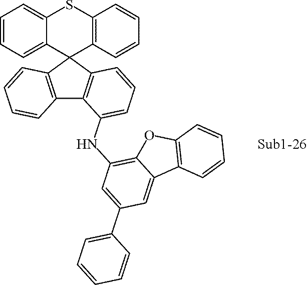 [SMILES: c1ccc(-c2cc(Nc3cccc4c3-c3ccccc3C43c4ccccc4Sc4ccccc43)c3oc4ccccc4c3c2)cc1]

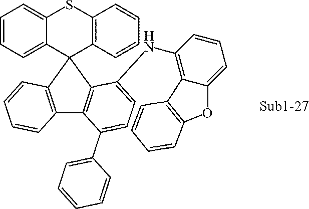 [SMILES: c1ccc(-c2ccc(Nc3cccc4oc5ccccc5c34)c3c2-c2ccccc2C32c3ccccc3Sc3ccccc32)cc1]

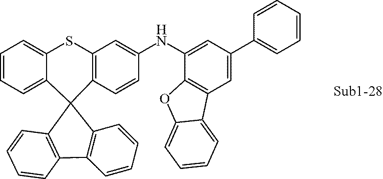 [SMILES: c1ccc(-c2cc(Nc3ccc4c(c3)Sc3ccccc3C43c4ccccc4-c4ccccc43)c3oc4ccccc4c3c2)cc1]

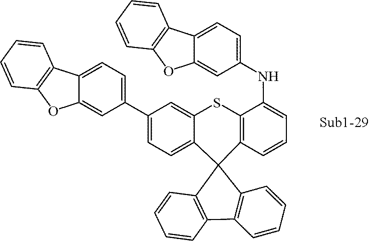 [SMILES: c1ccc2c(c1)-c1ccccc1C21c2ccc(-c3ccc4c(c3)oc3ccccc34)cc2Sc2c(Nc3ccc4c(c3)oc3ccccc34)cccc21]

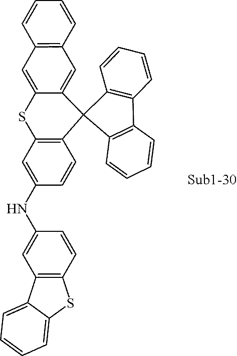 [SMILES: c1ccc2c(c1)-c1ccccc1C21c2ccc(Nc3ccc4sc5ccccc5c4c3)cc2Sc2cc3ccccc3cc21]

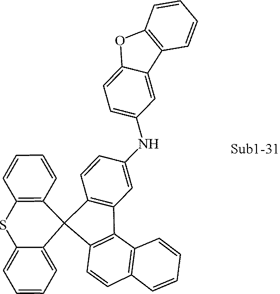 [SMILES: c1ccc2c(c1)Sc1ccccc1C21c2ccc(Nc3ccc4oc5ccccc5c4c3)cc2-c2c1ccc1ccccc21]

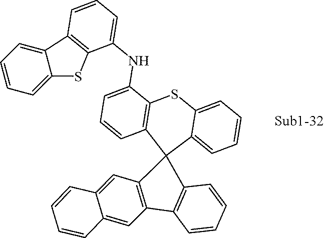 [SMILES: c1ccc2c(c1)Sc1c(Nc3cccc4c3sc3ccccc34)cccc1C21c2ccccc2-c2cc3ccccc3cc21]

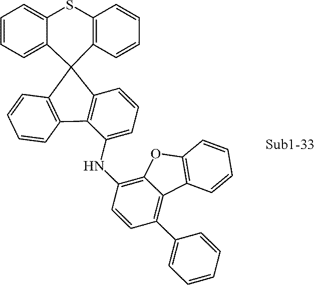 [SMILES: c1ccc(-c2ccc(Nc3cccc4c3-c3ccccc3C43c4ccccc4Sc4ccccc43)c3oc4ccccc4c23)cc1]

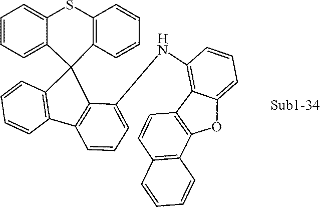 [SMILES: c1ccc2c(c1)Sc1ccccc1C21c2ccccc2-c2cccc(Nc3cccc4oc5c6ccccc6ccc5c34)c21]

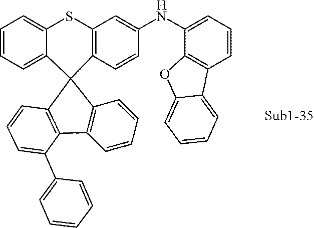 [SMILES: c1ccc(-c2cccc3c2-c2ccccc2C32c3ccccc3Sc3cc(Nc4cccc5c4oc4ccccc45)ccc32)cc1]

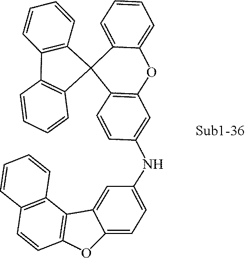 [SMILES: c1ccc2c(c1)Oc1cc(Nc3ccc4oc5ccc6ccccc6c5c4c3)ccc1C21c2ccccc2-c2ccccc21]

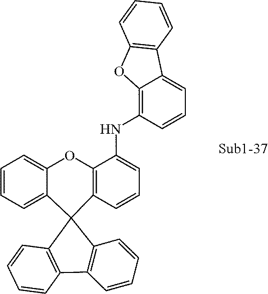 [SMILES: c1ccc2c(c1)Oc1c(Nc3cccc4c3oc3ccccc34)cccc1C21c2ccccc2-c2ccccc21]

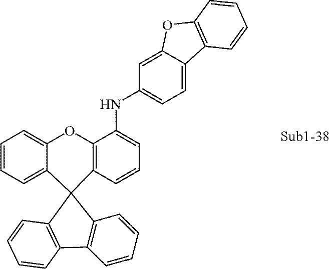 [SMILES: c1ccc2c(c1)Oc1c(Nc3ccc4c(c3)oc3ccccc34)cccc1C21c2ccccc2-c2ccccc21]

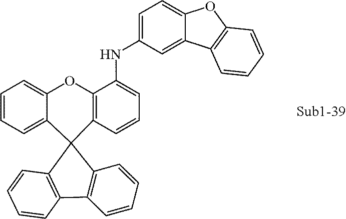 [SMILES: c1ccc2c(c1)Oc1c(Nc3ccc4oc5ccccc5c4c3)cccc1C21c2ccccc2-c2ccccc21]

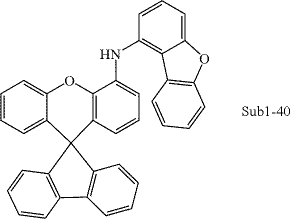 [SMILES: c1ccc2c(c1)Oc1c(Nc3cccc4oc5ccccc5c34)cccc1C21c2ccccc2-c2ccccc21]

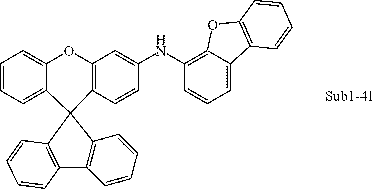 [SMILES: c1ccc2c(c1)Oc1cc(Nc3cccc4c3oc3ccccc34)ccc1C21c2ccccc2-c2ccccc21]

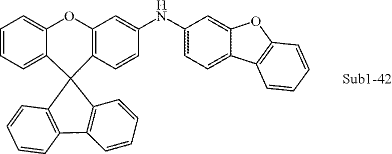 [SMILES: c1ccc2c(c1)Oc1cc(Nc3ccc4c(c3)oc3ccccc34)ccc1C21c2ccccc2-c2ccccc21]

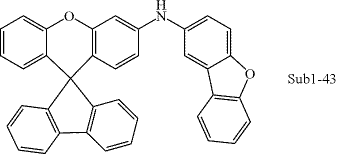 [SMILES: c1ccc2c(c1)Oc1cc(Nc3ccc4oc5ccccc5c4c3)ccc1C21c2ccccc2-c2ccccc21]

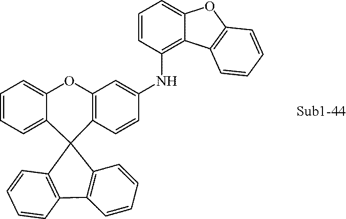 [SMILES: c1ccc2c(c1)Oc1cc(Nc3cccc4oc5ccccc5c34)ccc1C21c2ccccc2-c2ccccc21]

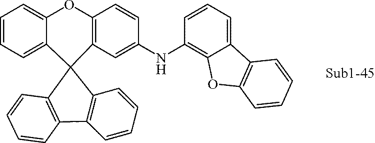 [SMILES: c1ccc2c(c1)Oc1ccc(Nc3cccc4c3oc3ccccc34)cc1C21c2ccccc2-c2ccccc21]

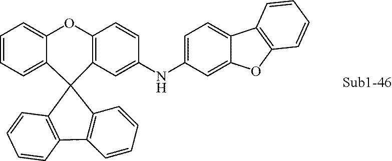 [SMILES: c1ccc2c(c1)Oc1ccc(Nc3ccc4c(c3)oc3ccccc34)cc1C21c2ccccc2-c2ccccc21]

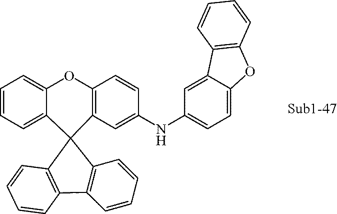 [SMILES: c1ccc2c(c1)Oc1ccc(Nc3ccc4oc5ccccc5c4c3)cc1C21c2ccccc2-c2ccccc21]

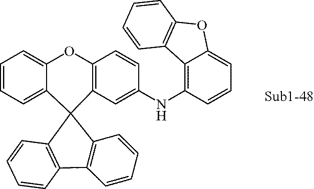 [SMILES: c1ccc2c(c1)Oc1ccc(Nc3cccc4oc5ccccc5c34)cc1C21c2ccccc2-c2ccccc21]

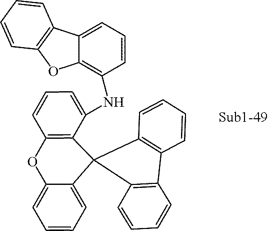 [SMILES: c1ccc2c(c1)Oc1cccc(Nc3cccc4c3oc3ccccc34)c1C21c2ccccc2-c2ccccc21]

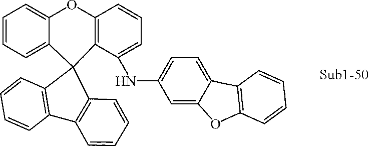 [SMILES: c1ccc2c(c1)Oc1cccc(Nc3ccc4c(c3)oc3ccccc34)c1C21c2ccccc2-c2ccccc21]

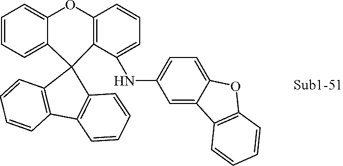 [SMILES: c1ccc2c(c1)Oc1cccc(Nc3ccc4oc5ccccc5c4c3)c1C21c2ccccc2-c2ccccc21]

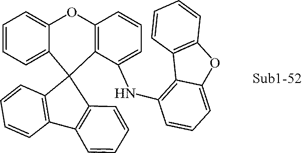 [SMILES: c1ccc2c(c1)Oc1cccc(Nc3cccc4oc5ccccc5c34)c1C21c2ccccc2-c2ccccc21]

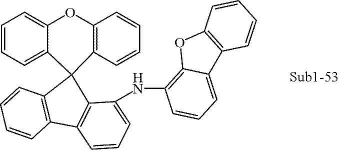 [SMILES: c1ccc2c(c1)Oc1ccccc1C21c2ccccc2-c2cccc(Nc3cccc4c3oc3ccccc34)c21]

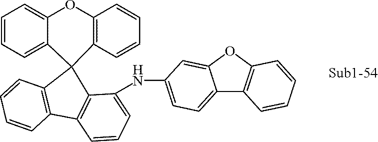 [SMILES: c1ccc2c(c1)Oc1ccccc1C21c2ccccc2-c2cccc(Nc3ccc4c(c3)oc3ccccc34)c21]

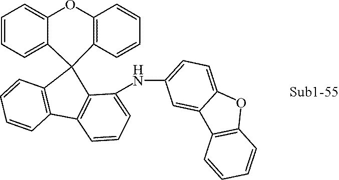 [SMILES: c1ccc2c(c1)Oc1ccccc1C21c2ccccc2-c2cccc(Nc3ccc4oc5ccccc5c4c3)c21]

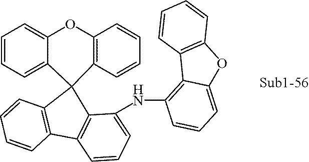 [SMILES: c1ccc2c(c1)Oc1ccccc1C21c2ccccc2-c2cccc(Nc3cccc4oc5ccccc5c34)c21]

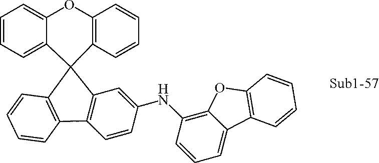 [SMILES: c1ccc2c(c1)Oc1ccccc1C21c2ccccc2-c2ccc(Nc3cccc4c3oc3ccccc34)cc21]

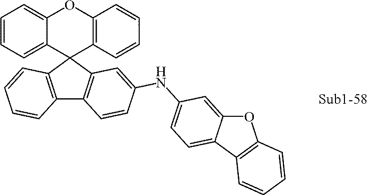 [SMILES: c1ccc2c(c1)Oc1ccccc1C21c2ccccc2-c2ccc(Nc3ccc4c(c3)oc3ccccc34)cc21]

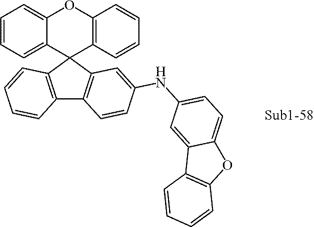 [SMILES: c1ccc2c(c1)Oc1ccccc1C21c2ccccc2-c2ccc(Nc3ccc4oc5ccccc5c4c3)cc21]

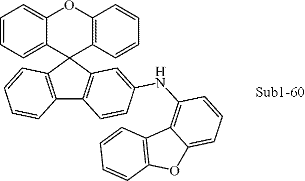 [SMILES: c1ccc2c(c1)Oc1ccccc1C21c2ccccc2-c2ccc(Nc3cccc4oc5ccccc5c34)cc21]

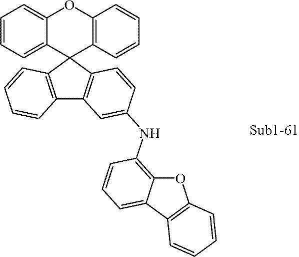 [SMILES: c1ccc2c(c1)Oc1ccccc1C21c2ccccc2-c2cc(Nc3cccc4c3oc3ccccc34)ccc21]

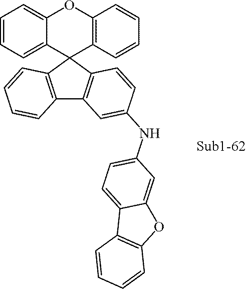 [SMILES: c1ccc2c(c1)Oc1ccccc1C21c2ccccc2-c2cc(Nc3ccc4c(c3)oc3ccccc34)ccc21]

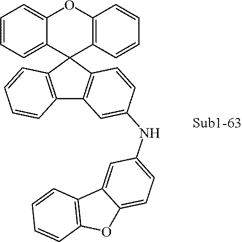 [SMILES: c1ccc2c(c1)Oc1ccccc1C21c2ccccc2-c2cc(Nc3ccc4oc5ccccc5c4c3)ccc21]

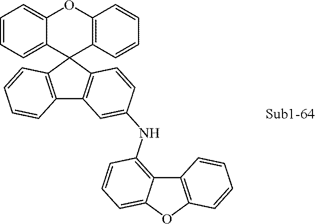 [SMILES: c1ccc2c(c1)Oc1ccccc1C21c2ccccc2-c2cc(Nc3cccc4oc5ccccc5c34)ccc21]

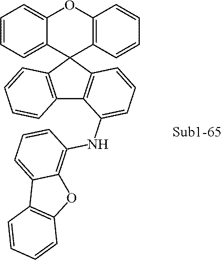 [SMILES: c1ccc2c(c1)Oc1ccccc1C21c2ccccc2-c2c(Nc3cccc4c3oc3ccccc34)cccc21]

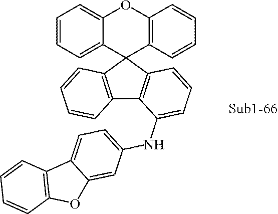 [SMILES: c1ccc2c(c1)Oc1ccccc1C21c2ccccc2-c2c(Nc3ccc4c(c3)oc3ccccc34)cccc21]

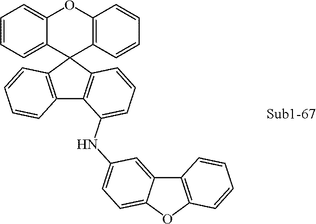 [SMILES: c1ccc2c(c1)Oc1ccccc1C21c2ccccc2-c2c(Nc3ccc4oc5ccccc5c4c3)cccc21]

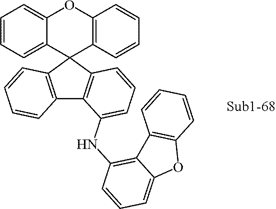 [SMILES: c1ccc2c(c1)Oc1ccccc1C21c2ccccc2-c2c(Nc3cccc4oc5ccccc5c34)cccc21]

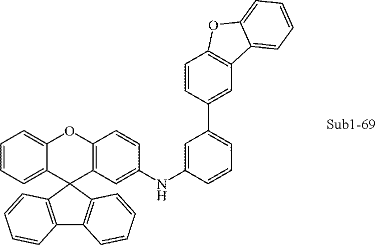 [SMILES: c1cc(Nc2ccc3c(c2)C2(c4ccccc4O3)c3ccccc3-c3ccccc32)cc(-c2ccc3oc4ccccc4c3c2)c1]

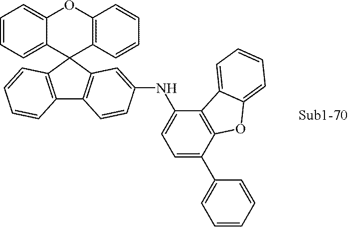 [SMILES: c1ccc(-c2ccc(Nc3ccc4c(c3)C3(c5ccccc5Oc5ccccc53)c3ccccc3-4)c3c2oc2ccccc23)cc1]

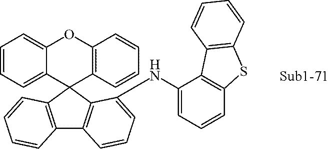 [SMILES: c1ccc2c(c1)Oc1ccccc1C21c2ccccc2-c2cccc(Nc3cccc4sc5ccccc5c34)c21]

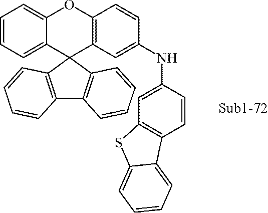 [SMILES: c1ccc2c(c1)Oc1ccc(Nc3ccc4c(c3)sc3ccccc34)cc1C21c2ccccc2-c2ccccc21]

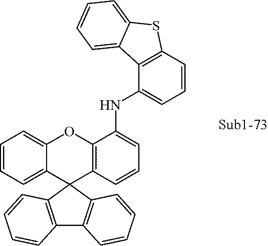 [SMILES: c1ccc2c(c1)Oc1c(Nc3cccc4sc5ccccc5c34)cccc1C21c2ccccc2-c2ccccc21]

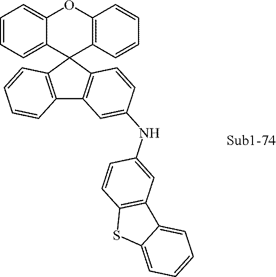 [SMILES: c1ccc2c(c1)Oc1ccccc1C21c2ccccc2-c2cc(Nc3ccc4sc5ccccc5c4c3)ccc21]

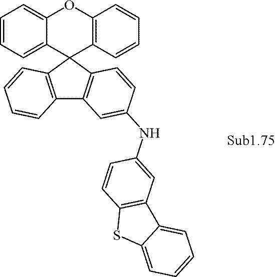 [SMILES: c1ccc2c(c1)Oc1ccccc1C21c2ccccc2-c2cc(Nc3ccc4sc5ccccc5c4c3)ccc21]

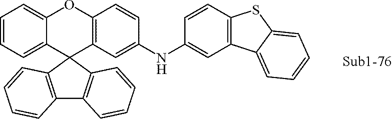 [SMILES: c1ccc2c(c1)Oc1ccc(Nc3ccc4sc5ccccc5c4c3)cc1C21c2ccccc2-c2ccccc21]

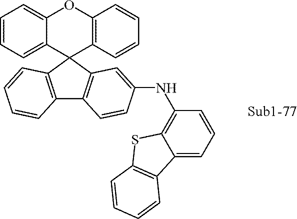 [SMILES: c1ccc2c(c1)Oc1ccccc1C21c2ccccc2-c2ccc(Nc3cccc4c3sc3ccccc34)cc21]

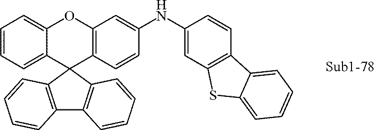 [SMILES: c1ccc2c(c1)Oc1cc(Nc3ccc4c(c3)sc3ccccc34)ccc1C21c2ccccc2-c2ccccc21]

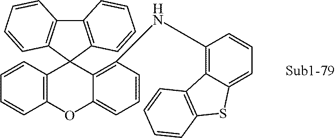 [SMILES: c1ccc2c(c1)Oc1cccc(Nc3cccc4sc5ccccc5c34)c1C21c2ccccc2-c2ccccc21]

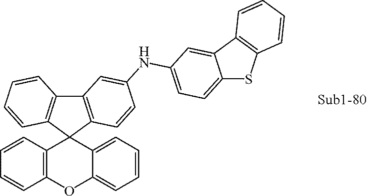 [SMILES: c1ccc2c(c1)Oc1ccccc1C21c2ccccc2-c2cc(Nc3ccc4sc5ccccc5c4c3)ccc21]

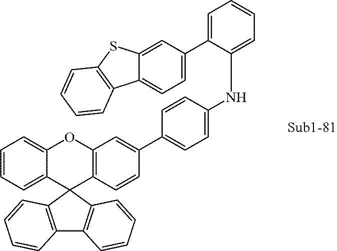 [SMILES: c1ccc(-c2ccc3c(c2)sc2ccccc23)c(Nc2ccc(-c3ccc4c(c3)Oc3ccccc3C43c4ccccc4-c4ccccc43)cc2)c1]

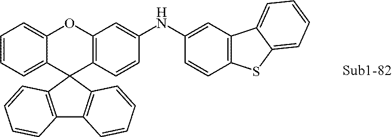 [SMILES: c1ccc2c(c1)Oc1cc(Nc3ccc4sc5ccccc5c4c3)ccc1C21c2ccccc2-c2ccccc21]

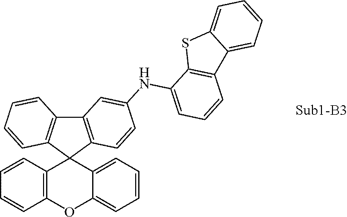 [SMILES: c1ccc2c(c1)Oc1ccccc1C21c2ccccc2-c2cc(Nc3cccc4c3sc3ccccc34)ccc21]

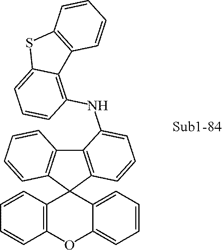 [SMILES: c1ccc2c(c1)Oc1ccccc1C21c2ccccc2-c2c(Nc3cccc4sc5ccccc5c34)cccc21]

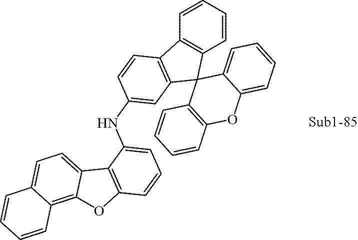 [SMILES: c1ccc2c(c1)Oc1ccccc1C21c2ccccc2-c2ccc(Nc3cccc4oc5c6ccccc6ccc5c34)cc21]

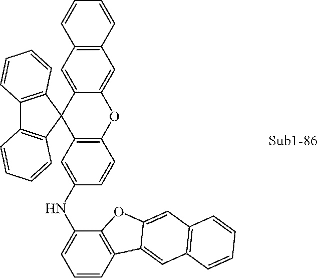 [SMILES: c1ccc2c(c1)-c1ccccc1C21c2cc(Nc3cccc4c3oc3cc5ccccc5cc34)ccc2Oc2cc3ccccc3cc21]

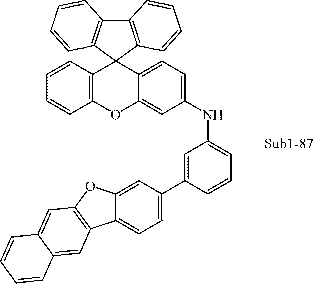 [SMILES: c1cc(Nc2ccc3c(c2)Oc2ccccc2C32c3ccccc3-c3ccccc32)cc(-c2ccc3c(c2)oc2cc4ccccc4cc23)c1]

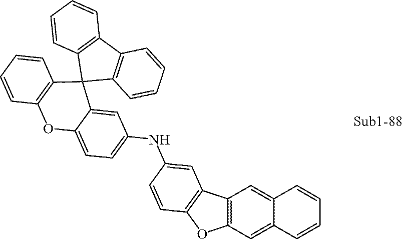 [SMILES: c1ccc2c(c1)Oc1ccc(Nc3ccc4oc5cc6ccccc6cc5c4c3)cc1C21c2ccccc2-c2ccccc21]

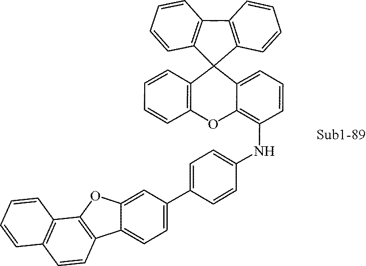 [SMILES: c1ccc2c(c1)Oc1c(Nc3ccc(-c4ccc5c(c4)oc4c6ccccc6ccc54)cc3)cccc1C21c2ccccc2-c2ccccc21]

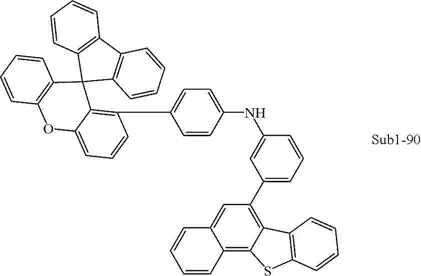 [SMILES: c1cc(Nc2ccc(-c3cccc4c3C3(c5ccccc5O4)c4ccccc4-c4ccccc43)cc2)cc(-c2cc3ccccc3c3sc4ccccc4c23)c1]

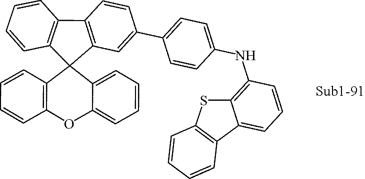 [SMILES: c1ccc2c(c1)Oc1ccccc1C21c2ccccc2-c2ccc(-c3ccc(Nc4cccc5c4sc4ccccc45)cc3)cc21]

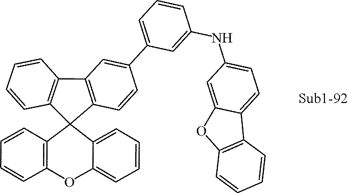 [SMILES: c1cc(Nc2ccc3c(c2)oc2ccccc23)cc(-c2ccc3c(c2)-c2ccccc2C32c3ccccc3Oc3ccccc32)c1]

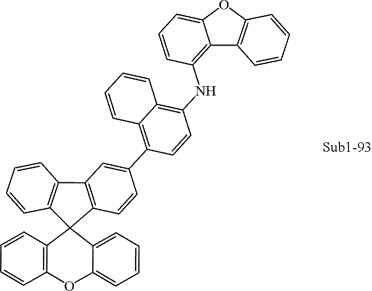 [SMILES: c1ccc2c(c1)Oc1ccccc1C21c2ccccc2-c2cc(-c3ccc(Nc4cccc5oc6ccccc6c45)c4ccccc34)ccc21]

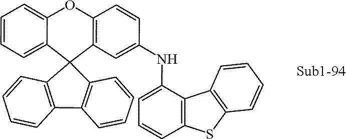 [SMILES: c1ccc2c(c1)Oc1ccc(Nc3cccc4sc5ccccc5c34)cc1C21c2ccccc2-c2ccccc21]

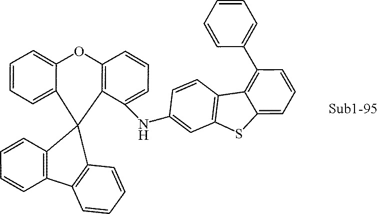 [SMILES: c1ccc(-c2cccc3sc4cc(Nc5cccc6c5C5(c7ccccc7O6)c6ccccc6-c6ccccc65)ccc4c23)cc1]

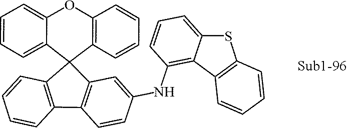 [SMILES: c1ccc2c(c1)Oc1ccccc1C21c2ccccc2-c2ccc(Nc3cccc4sc5ccccc5c34)cc21]

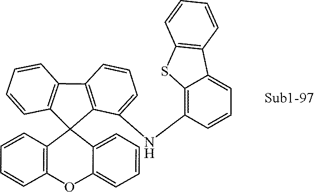 [SMILES: c1ccc2c(c1)Oc1ccccc1C21c2ccccc2-c2cccc(Nc3cccc4c3sc3ccccc34)c21]

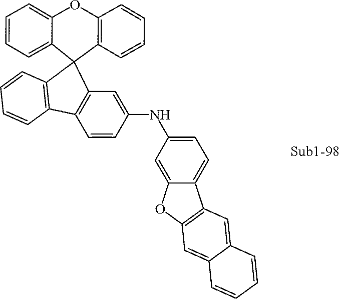 [SMILES: c1ccc2c(c1)Oc1ccccc1C21c2ccccc2-c2ccc(Nc3ccc4c(c3)oc3cc5ccccc5cc34)cc21]

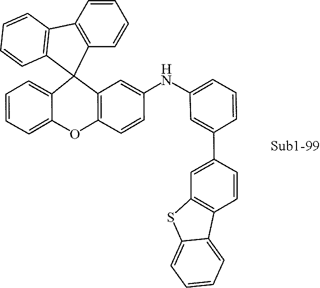 [SMILES: c1cc(Nc2ccc3c(c2)C2(c4ccccc4O3)c3ccccc3-c3ccccc32)cc(-c2ccc3c(c2)sc2ccccc23)c1]

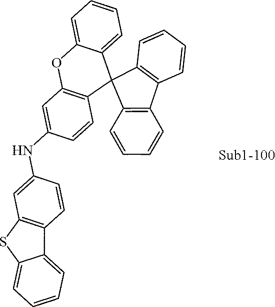 [SMILES: c1ccc2c(c1)Oc1cc(Nc3ccc4c(c3)sc3ccccc34)ccc1C21c2ccccc2-c2ccccc21]

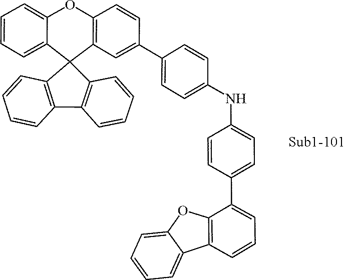 [SMILES: c1ccc2c(c1)Oc1ccc(-c3ccc(Nc4ccc(-c5cccc6c5oc5ccccc56)cc4)cc3)cc1C21c2ccccc2-c2ccccc21]

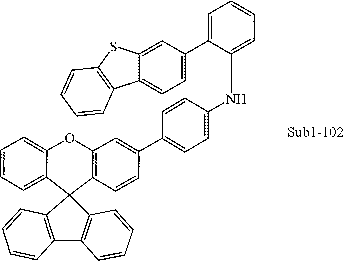 [SMILES: c1ccc(-c2ccc3c(c2)sc2ccccc23)c(Nc2ccc(-c3ccc4c(c3)Oc3ccccc3C43c4ccccc4-c4ccccc43)cc2)c1]

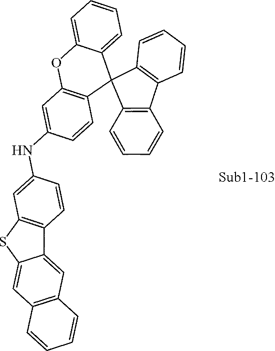 [SMILES: c1ccc2c(c1)Oc1cc(Nc3ccc4c(c3)sc3cc5ccccc5cc34)ccc1C21c2ccccc2-c2ccccc21]

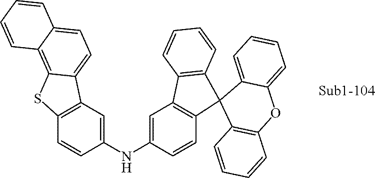 [SMILES: c1ccc2c(c1)Oc1ccccc1C21c2ccccc2-c2cc(Nc3ccc4sc5c6ccccc6ccc5c4c3)ccc21]

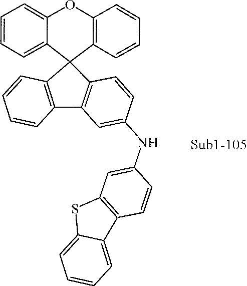 [SMILES: c1ccc2c(c1)Oc1ccccc1C21c2ccccc2-c2cc(Nc3ccc4c(c3)sc3ccccc34)ccc21]

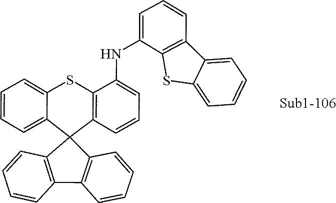 [SMILES: c1ccc2c(c1)Sc1c(Nc3cccc4c3sc3ccccc34)cccc1C21c2ccccc2-c2ccccc21]

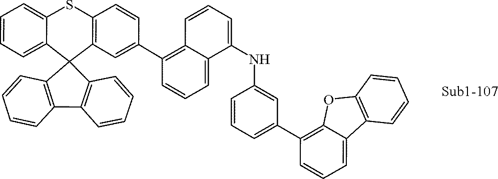 [SMILES: c1cc(Nc2cccc3c(-c4ccc5c(c4)C4(c6ccccc6S5)c5ccccc5-c5ccccc54)cccc23)cc(-c2cccc3c2oc2ccccc23)c1]

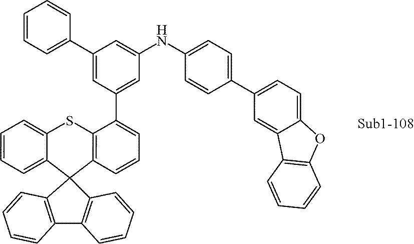 [SMILES: c1ccc(-c2cc(Nc3ccc(-c4ccc5oc6ccccc6c5c4)cc3)cc(-c3cccc4c3Sc3ccccc3C43c4ccccc4-c4ccccc43)c2)cc1]

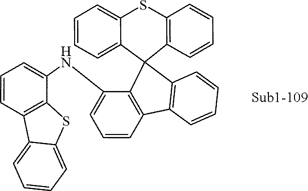 [SMILES: c1ccc2c(c1)Sc1ccccc1C21c2ccccc2-c2cccc(Nc3cccc4c3sc3ccccc34)c21]

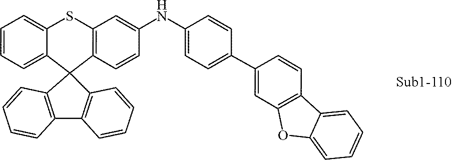 [SMILES: c1ccc2c(c1)Sc1cc(Nc3ccc(-c4ccc5c(c4)oc4ccccc45)cc3)ccc1C21c2ccccc2-c2ccccc21]

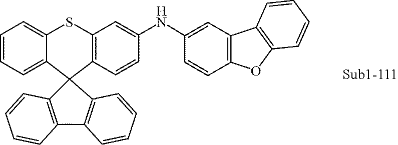 [SMILES: c1ccc2c(c1)Sc1cc(Nc3ccc4oc5ccccc5c4c3)ccc1C21c2ccccc2-c2ccccc21]

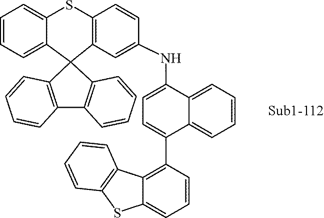 [SMILES: c1ccc2c(c1)Sc1ccc(Nc3ccc(-c4cccc5sc6ccccc6c45)c4ccccc34)cc1C21c2ccccc2-c2ccccc21]

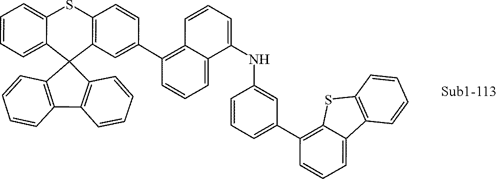 [SMILES: c1cc(Nc2cccc3c(-c4ccc5c(c4)C4(c6ccccc6S5)c5ccccc5-c5ccccc54)cccc23)cc(-c2cccc3c2sc2ccccc23)c1]

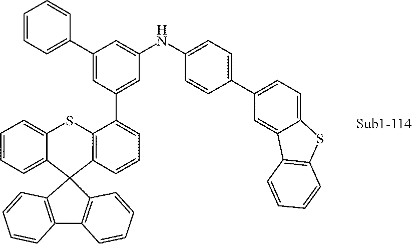 [SMILES: c1ccc(-c2cc(Nc3ccc(-c4ccc5sc6ccccc6c5c4)cc3)cc(-c3cccc4c3Sc3ccccc3C43c4ccccc4-c4ccccc43)c2)cc1]

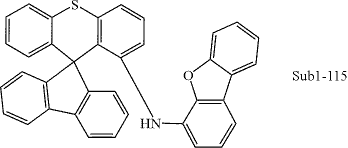 [SMILES: c1ccc2c(c1)Sc1cccc(Nc3cccc4c3oc3ccccc34)c1C21c2ccccc2-c2ccccc21]

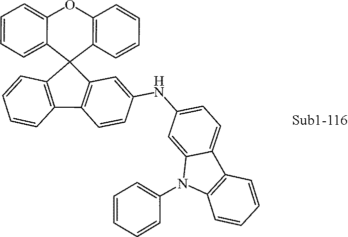 [SMILES: c1ccc(-n2c3ccccc3c3ccc(Nc4ccc5c(c4)C4(c6ccccc6Oc6ccccc64)c4ccccc4-5)cc32)cc1]

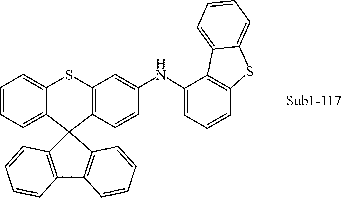 [SMILES: c1ccc2c(c1)Sc1cc(Nc3cccc4sc5ccccc5c34)ccc1C21c2ccccc2-c2ccccc21]

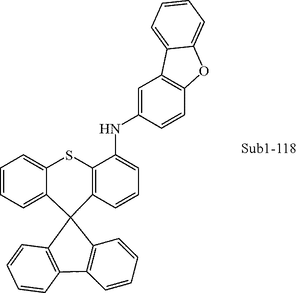 [SMILES: c1ccc2c(c1)Sc1c(Nc3ccc4oc5ccccc5c4c3)cccc1C21c2ccccc2-c2ccccc21]

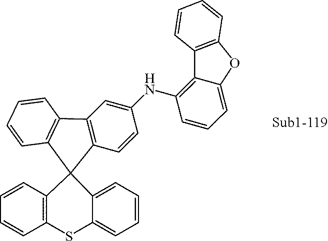 [SMILES: c1ccc2c(c1)Sc1ccccc1C21c2ccccc2-c2cc(Nc3cccc4oc5ccccc5c34)ccc21]

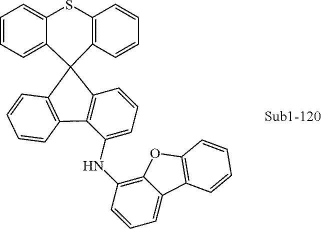 [SMILES: c1ccc2c(c1)Sc1ccccc1C21c2ccccc2-c2c(Nc3cccc4c3oc3ccccc34)cccc21]

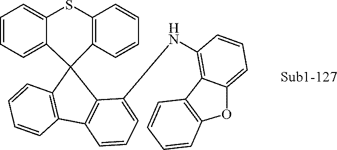 [SMILES: c1ccc2c(c1)Sc1ccccc1C21c2ccccc2-c2cccc(Nc3cccc4oc5ccccc5c34)c21]

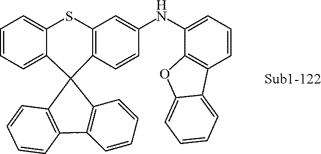 [SMILES: c1ccc2c(c1)Sc1cc(Nc3cccc4c3oc3ccccc34)ccc1C21c2ccccc2-c2ccccc21]

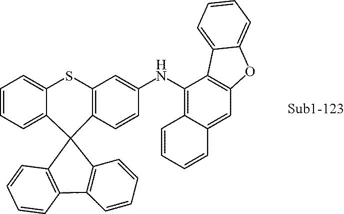 [SMILES: c1ccc2c(c1)Sc1cc(Nc3c4ccccc4cc4oc5ccccc5c34)ccc1C21c2ccccc2-c2ccccc21]

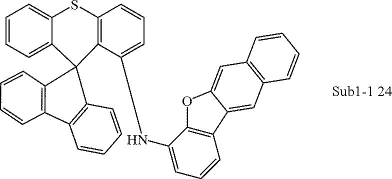 [SMILES: c1ccc2c(c1)Sc1cccc(Nc3cccc4c3oc3cc5ccccc5cc34)c1C21c2ccccc2-c2ccccc21]

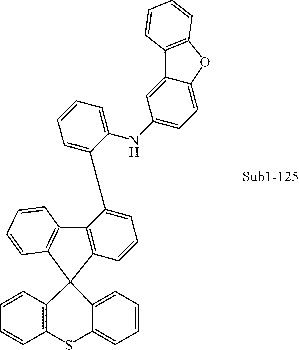 [SMILES: c1ccc(-c2cccc3c2-c2ccccc2C32c3ccccc3Sc3ccccc32)c(Nc2ccc3oc4ccccc4c3c2)c1]

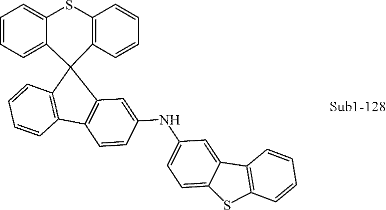 [SMILES: c1ccc2c(c1)Sc1ccccc1C21c2ccccc2-c2ccc(Nc3ccc4sc5ccccc5c4c3)cc21]

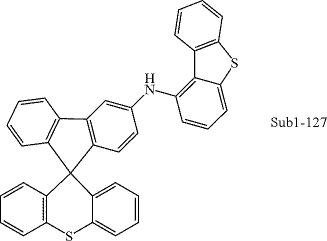 [SMILES: c1ccc2c(c1)Sc1ccccc1C21c2ccccc2-c2cc(Nc3cccc4sc5ccccc5c34)ccc21]

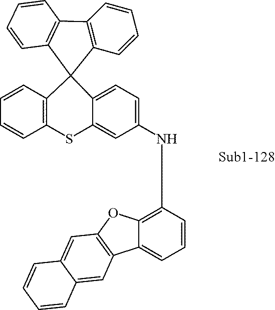 [SMILES: c1ccc2c(c1)Sc1cc(Nc3cccc4c3oc3cc5ccccc5cc34)ccc1C21c2ccccc2-c2ccccc21]

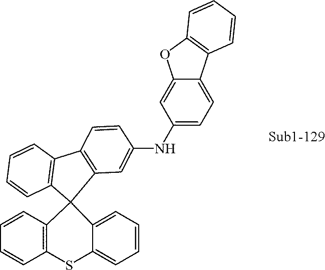 [SMILES: c1ccc2c(c1)Sc1ccccc1C21c2ccccc2-c2ccc(Nc3ccc4c(c3)oc3ccccc34)cc21]

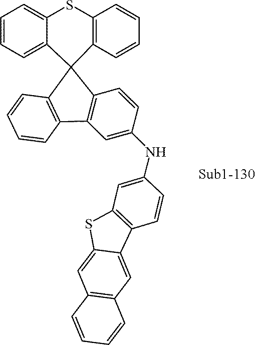 [SMILES: c1ccc2c(c1)Sc1ccccc1C21c2ccccc2-c2cc(Nc3ccc4c(c3)sc3cc5ccccc5cc34)ccc21]

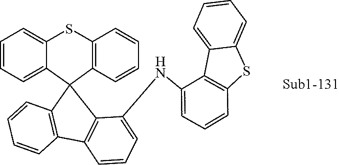 [SMILES: c1ccc2c(c1)Sc1ccccc1C21c2ccccc2-c2cccc(Nc3cccc4sc5ccccc5c34)c21]

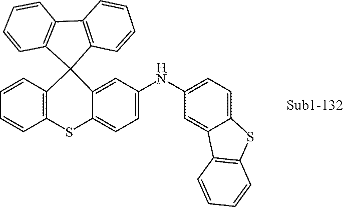 [SMILES: c1ccc2c(c1)Sc1ccc(Nc3ccc4sc5ccccc5c4c3)cc1C21c2ccccc2-c2ccccc21]

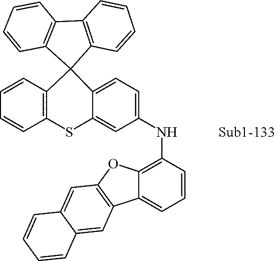 [SMILES: c1ccc2c(c1)Sc1cc(Nc3cccc4c3oc3cc5ccccc5cc34)ccc1C21c2ccccc2-c2ccccc21]

TABLE 1

| | | | |
|---|---|---|---|
| Compound | FD-MS | Compound | FD-MS |
| Sub 1-1 | m/z=545.13(C₃₇H₂₃NS₂=545.72) | Sub 1-2 | m/z=588.22(C₄₃H₂₈N₂O=588.71) |
| Sub 1-3 | m/z=588.22(C₄₃H₂₈N₂O=588.71) | Sub 1-4 | m/z=588.22(C₄₃H₂₈N₂O=588.71) |
| Sub 1-5 | m/z=588.22(C₄₃H₂₈N₂O=588.71) | Sub 1-6 | m/z=588.22(C₄₃H₂₈N₂O=588.71) |
| Sub 1-7 | m/z=588.22(C₄₃H₂₈N₂O=588.71) | Sub 1-8 | m/z=604.2(C₄₃H₂₈N₂S=604.77) |
| Sub 1-9 | m/z=604.2(C₄₃H₂₈N₂S=604.77) | Sub 1-10 | m/z=604.2(C₄₃H₂₈N₂S=604.77) |
| Sub 1-11 | m/z=604.2(C₄₃H₂₈N₂S=604.77) | Sub 1-12 | m/z=604.2(C₄₃H₂₈N₂S=604.77) |
| Sub 1-13 | m/z=604.2(C₄₃H₂₈N₂S=604.77) | Sub 1-14 | m/z=604.2(C₄₃H₂₈N₂S=604.77) |
| Sub 1-15 | m/z=589.2(C₄₃H₂₇NO₂=589.69) | Sub 1-16 | m/z=589.2(C₄₃H₂₇NO₂=589.69) |
| Sub 1-17 | m/z=589.2(C₄₃H₂₇NO₂=589.69) | Sub 1-18 | m/z=589.2(C₄₃H₂₇NO₂=589.69) |
| Sub 1-19 | m/z=589.2(C₄₃H₂₇NO₂=589.69) | Sub 1-20 | m/z=589.2(C₄₃H₂₇NO₂=589.69) |
| Sub 1-21 | m/z=589.2(C₄₃H₂₇NO₂=589.69) | Sub 1-22 | m/z=605.18(C₄₃H₂₇NOS=605.76) |
| Sub 1-23 | m/z=605.18(C₄₃H₂₇NOS=605.76) | Sub 1-24 | m/z=605.18(C₄₃H₂₇NOS=605.76) |
| Sub 1-25 | m/z=605.18(C₄₃H₂₇NOS=605.76) | Sub 1-26 | m/z=605.18(C₄₃H₂₇NOS=605.76) |
| Sub 1-27 | m/z=605.18(C₄₃H₂₇NOS=605.76) | Sub 1-28 | m/z=605.18(C₄₃H₂₇NOS=605.76) |
| Sub 1-29 | m/z=695.19(C₄₉H₂₉NO₂S=695.84) | Sub 1-30 | m/z=595.14(C₄₁H₂₅NS₂=595.78) |
| Sub 1-31 | m/z=579.17(C₄₁H₂₅NOS=579.72) | Sub 1-32 | m/z=595.14(C₄₁H₂₅NS₂=595.78) |
| Sub 1-33 | m/z=605.18(C₄₃H₂₇NOS=605.76) | Sub 1-34 | m/z=579.17(C₄₁H₂₅NOS=579.72) |
| Sub 1-35 | m/z=605.18(C₄₃H₂₇NOS=605.76) | Sub 1-36 | m/z=563.19(C₄₁H₂₅NO₂=563.66) |
| Sub 1-37 | m/z=513.17(C₃₇H₂₃NO₂=513.6) | Sub 1-38 | m/z=513.17(C₃₇H₂₃NO₂=513.6) |
| Sub 1-39 | m/z=513.17(C₃₇H₂₃NO₂=513.6) | Sub 1-40 | m/z=513.17(C₃₇H₂₃NO₂=513.6) |
| Sub 1-41 | m/z=513.17(C₃₇H₂₃NO₂=513.6) | Sub 1-42 | m/z=513.17(C₃₇H₂₃NO₂=513.6) |
| Sub 1-43 | m/z=513.17(C₃₇H₂₃NO₂=513.6) | Sub 1-44 | m/z=513.17(C₃₇H₂₃NO₂=513.6) |
| Sub 1-45 | m/z=513.17(C₃₇H₂₃NO₂=513.6) | Sub 1-46 | m/z=513.17(C₃₇H₂₃NO₂=513.6) |
| Sub 1-47 | m/z=513.17(C₃₇H₂₃NO₂=513.6) | Sub 1-48 | m/z=513.17(C₃₇H₂₃NO₂=513.6) |
| Sub 1-49 | m/z=513.17(C₃₇H₂₃NO₂=513.6) | Sub 1-50 | m/z=513.17(C₃₇H₂₃NO₂=513.6) |
| Sub 1-51 | m/z=513.17(C₃₇H₂₃NO₂=513.6) | Sub 1-52 | m/z=513.17(C₃₇H₂₃NO₂=513.6) |
| Sub 1-53 | m/z=513.17(C₃₇H₂₃NO₂=513.6) | Sub 1-54 | m/z=513.17(C₃₇H₂₃NO₂=513.6) |
| Sub 1-55 | m/z=513.17(C₃₇H₂₃NO₂=513.6) | Sub 1-56 | m/z=513.17(C₃₇H₂₃NO₂=513.6) |
| Sub 1-57 | m/z=513.17(C₃₇H₂₃NO₂=513.6) | Sub 1-58 | m/z=513.17(C₃₇H₂₃NO₂=513.6) |
| Sub 1-59 | m/z=513.17(C₃₇H₂₃NO₂=513.6) | Sub 1-60 | m/z=513.17(C₃₇H₂₃NO₂=513.6) |
| Sub 1-61 | m/z=513.17(C₃₇H₂₃NO₂=513.6) | Sub 1-62 | m/z=513.17(C₃₇H₂₃NO₂=513.6) |
| Sub 1-63 | m/z=513.17(C₃₇H₂₃NO₂=513.6) | Sub 1-64 | m/z=513.17(C₃₇H₂₃NO₂=513.6) |
| Sub 1-65 | m/z=513.17(C₃₇H₂₃NO₂=513.6) | Sub 1-66 | m/z=513.17(C₃₇H₂₃NO₂=513.6) |
| Sub 1-67 | m/z=513.17(C₃₇H₂₃NO₂=513.6) | Sub 1-68 | m/z=513.17(C₃₇H₂₃NO₂=513.6) |
| Sub 1-69 | m/z=589.2(C₄₃H₂₇NO₂=589.69) | Sub 1-70 | m/z=589.2(C₄₃H₂₇NO₂=589.69) |
| Sub 1-71 | m/z=529.15(C₃₇H₂₃NOS=529.66) | Sub 1-72 | m/z=529.15(C₃₇H₂₃NOS=529.66) |
| Sub 1-73 | m/z=529.15(C₃₇H₂₃NOS=529.66) | Sub 1-74 | m/z=529.15(C₃₇H₂₃NOS=529.66) |
| Sub 1-75 | m/z=529.15(C₃₇H₂₃NOS=529.66) | Sub 1-76 | m/z=529.15(C₃₇H₂₃NOS=529.66) |
| Sub 1-77 | m/z=529.15(C₃₇H₂₃NOS=529.66) | Sub 1-78 | m/z=529.15(C₃₇H₂₃NOS=529.66) |
| Sub 1-79 | m/z=529.15(C₃₇H₂₃NOS=529.66) | Sub 1-80 | m/z=529.15(C₃₇H₂₃NOS=529.66) |
| Sub 1-81 | m/z=681.21(C₄₉H₃₁NOS=681.85) | Sub 1-82 | m/z=529.15(C₃₇H₂₃NOS=529.66) |
| Sub 1-83 | m/z=529.15(C₃₇H₂₃NOS=529.66) | Sub 1-84 | m/z=529.15(C₃₇H₂₃NOS=529.66) |
| Sub 1-85 | m/z=563.19(C₄₁H₂₅NO₂=563.66) | Sub 1-86 | m/z=613.2(C₄₃H₂₇NO₂=613.72) |
| Sub 1-87 | iWz=639.22(C₄₇H₂₉NO₂=639.75) | Sub 1-88 | m/z=563.19(C₄₁H₂₅NO₂=563.66) |
| Sub 1-89 | m/z=639.22(C₄₇H₂₉NO₂=639.75) | Sub 1-90 | mlz=731.23(C₅₃H₃₃NOS=731.91) |
| Sub 1-91 | m/z=605.18(C₄₃H₂₇NOS=605.76) | Sub 1-92 | m/z=589.2(C₄₃H₂₇NO₂=589.69) |
| Sub 1-93 | m/z=639.22(C₄₇H₂₉NO₂=639.75) | Sub 1-94 | m/z=529.15(C₃₇H₂₃NOS=529.66) |
| Sub 1-95 | m/z=605.18(C₄₃H₂₇NOS=605.76) | Sub 1-96 | m/z=529.15(C₃₇H₂₃NOS=529.66) |
| Sub 1-97 | m/z=529.15(C₃₇H₂₃NOS=529.66) | Sub 1-98 | m/z=563.19(C₄₁H₂₅NO₂=563.66) |
| Sub 1-99 | m/z=605.18(C₄₃H₂₇NOS=605.76) | Sub 1-100 | m/z=529.15(C₃₇H₂₃NOS=529.66) |
| Sub 1-101 | m/z=665.24(C₄₉H₃₁NO₂=665.79) | Sub 1-102 | m/z=681.21(C₄₉H₃₁NOS=681.85) |
| Sub 1-103 | m/z=579.17(C₄₁H₂₅NOS=579.72) | Sub 1-104 | m/z=579.17(C₄₁H₂₅NOS=579.72) |
| Sub 1-105 | m/z=545.13(C₃₇H₂₃NS₂=545.72) | Sub 1-106 | m/z=545.13(C₃₇H₂₃NS₂=545.72) |
| Sub 1-107 | m/z=731.23(C₅₃H₃₃NOS=731.91) | Sub 1-108 | m/z=757.24(C₅₅H₃₅NOS=757.95) |
| Sub 1-109 | m/z=545.13(C₃₇H₂₃NS₂=545.72) | Sub 1-110 | m/z=605.18(C₄₃H₂₇NOS=605.76) |
| Sub 1-111 | m/z=529.15(C₃₇H₂₃NOS=529.66) | Sub 1-112 | m/z=671.17(C₄₇H₂₉NS₂=671.88) |
| Sub 1-113 | m/z=747.21(C₅₃H₃₃NS₂=747.97) | Sub 1-114 | m/z=773.22(C₃₃H₃₅NS₂=774.01) |
| Sub 1-115 | m/z=529.15(C₃₇H₂₃NOS=529.66) | Sub 1-116 | m/z=588.22(C₄₃H₂₈N₂O=588.71) |
| Sub 1-117 | m/z=545.13(C₃₇H₂₃NS₂=545.72) | Sub 1-118 | m/z=529.15(C₃₇H₂₃NOS=529.66) |
| Sub 1-119 | m/z=529.15(C₃₇H₂₃NOS=529.66) | Sub 1-120 | m/z=529.15(C₃₇H₂₃NOS=529.66) |
| Sub 1-121 | m/z=529.15(C₃₇H₂₃NOS=529.66) | Sub 1-122 | m/z=529.15(C₃₇H₂₃NOS=529.66) |
| Sub 1-123 | m/z=579.17(C₄₁H₂₅NOS=579.72) | Sub 1-124 | m/z=579.17(C₄₁H₂₅NOS=579.72) |
| Sub 1-125 | m/z=605.18(C₄₃H₂₇NOS=605.76) | Sub 1-126 | m/z=545.13(C₃₇H₂₃NS₂=545.72) |
| Sub 1-127 | m/z=545.13(C₃₇H₂₃NS₂=545.72) | Sub 1-128 | m/z=579.17(C₄₁H₂₅NOS=579.72) |
| Sub 1-129 | m/z=529.15(C₃₇H₂₃NOS=529.66) | Sub 1-130 | m/z=595.14(C₄₁H₂₅NS₂=595.78) |
| Sub 1-131 | m/z=545.13(C₃₇H₂₃NS₂=545.72) | Sub 1-132 | m/z=545.13(C₃₇H₂₃NS₂=545.72) |
| Sub 1-133 | m/z=579.17(C₄₁H₂₅NOS=579.72) | | |

### Synthesis Example of the Final Compound

### 1. Synthesis Example of 1-1

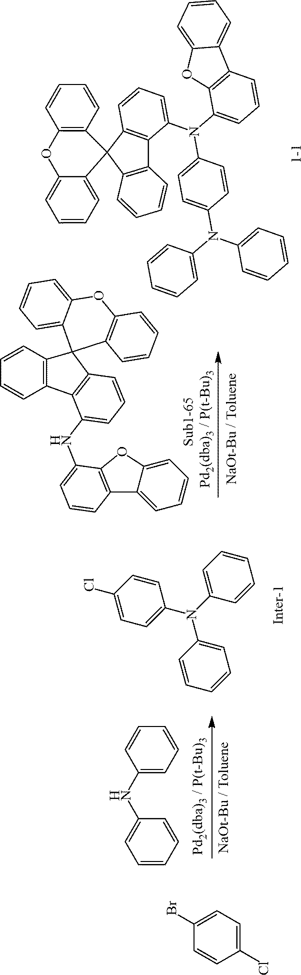

### Synthesis Example of Inter-1

After dissolving 1-bromo-4-chlorobenzene (7.66 g, 40 mmol) in toluene (150 mL), diphenylamine(7.45 g,44.01 mmol), Pd₂(dba)₃ (1.8 g, 2 mmol), P(t-Bu)₃(0.8 g, 4 mmol) and NaOt-Bu (11.5 g, 120 mmol) were added thereto and the mixture was stirred at 60° C. When the reaction was completed, the reaction product was extracted with CH₂Cl₂ and water, and the organic layer is dried with MgSO₄ and concentrated. Thereafter, the concentrate was separated by a silica gel column and recrystallized to obtain 9.9 g (yield: 89%) of the product.

### Synthesis Example of 1-1

After dissolving Inter-1 (5.6 g, 20 mmol) in toluene (25 mL), Sub1-65(11.3 g,22 mmol), Pd₂(dba)₃ (0.9 g, 1 mmol), P(t-Bu)₃(0.4 g, 2 mmol) and NaOt-Bu (5.8 g, 60 mmol) were added thereto and the mixture was refluxed. When the reaction was completed, the reaction product was extracted with CH₂Cl₂ and water, and the organic layer is dried with MgSO₄ and concentrated. Thereafter, the concentrate was separated by a silica gel column and recrystallized to obtain 12 g (yield: 79%) of the product.

### 2. Synthesis Example of 1-29

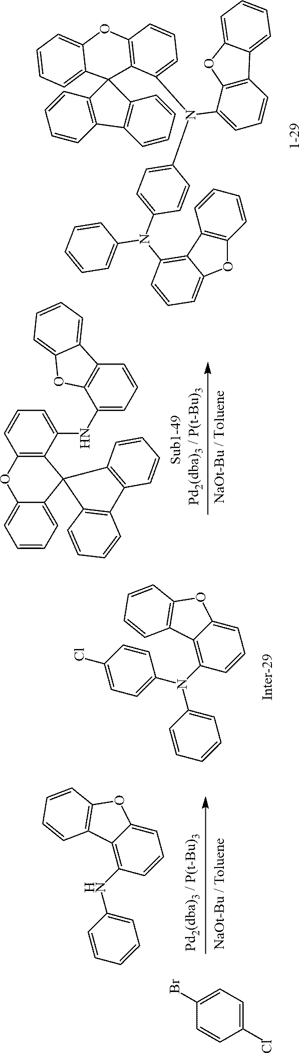

### Synthesis Example of Inter-29

The reaction was carried out in the same manner as in the synthesis method of Inter-1 using 1-bromo-4-chlorobenzene(7.6 g, 40 mmol), N-phenyldibenzo[b,d]furan-1-amine(11.4 g,44 mmol), Pd₂(dba)₃ (1.8 g, 2 mmol), P(t-Bu)₃(0.8 g, 4 mmol) and NaOt-Bu (11.5 g, 120 mmol) to obtain 22.4 g (yield: 74%) of product.

### Synthesis Example of 1-29

The reaction was carried out in the same manner as in the synthesis method of 1-1 using Inter-29 (5.6 g, 20 mmol), Subl-49(11.3 g, 22 mmol), Pd₂(dba)₃ (0.9 g, 1 mmol), P(t-Bu)₃(0.4 g, 2 mmol) and NaOt-Bu (5.8 g, 60 mmol) to obtain 14.2 g (yield: 84%) of product.

### 3. Synthesis Example of 1-56

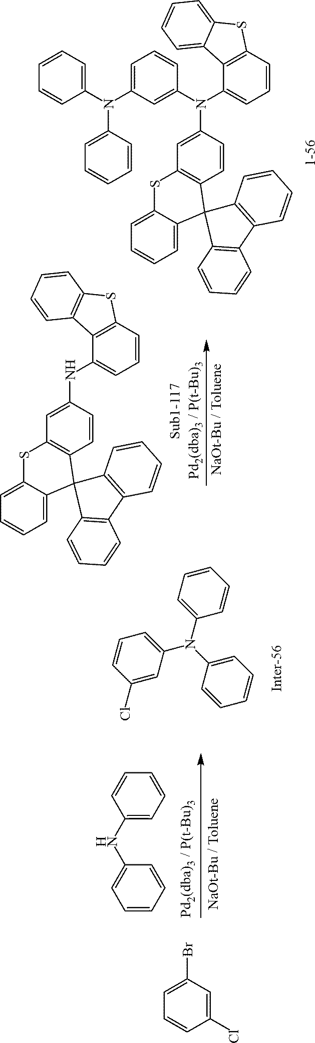

### Synthesis Example of Inter-56

The reaction was carried out in the same manner as in the synthesis method of Inter-1 using 1-bromo-3-chlorobenzene(7.6 g, 40 mmol), diphenylamine(7.4 g, 44 mmol), Pd₂(dba)₃ (1.8 g, 2 mmol), P(t-Bu)₃(0.8 g, 4 mmol) and NaOt-Bu (11.5 g, 120 mmol) to obtain 4.9 g (yield: 87%) of product.

### Synthesis Example of 1-56

The reaction was carried out in the same manner as in the synthesis method of 1-1 using Inter-56 (5.6 g, 20 mmol), Sub1-117(12 g, 22 mmol), Pd₂(dba)₃ (0.9 g, 1 mmol), P(t-Bu)₃(0.4 g, 2 mmol) and NaOt-Bu (5.8 g, 60 mmol) to obtain 11.7 g (yield: 74%) of product.

### 4. Synthesis Example of 1-80

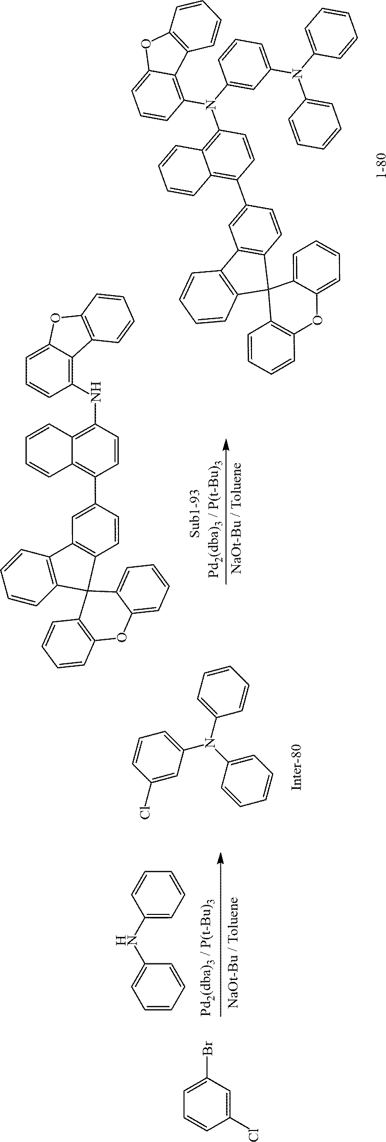

### Synthesis Example of Inter-80

The reaction was carried out in the same manner as in the synthesis method of Inter-1 using 1-bromo-3-chlorobenzene(7.6 g, 40 mmol), diphenylamine(7.4 g, 44 mmol), Pd₂(dba)₃ (1.8 g, 2 mmol), P(t-Bu)₃(0.8 g, 4 mmol) and NaOt-Bu (11.5 g, 120 mmol) to obtain 4.9 g (yield: 87%) of product.

### Synthesis Example of 1-80

The reaction was carried out in the same manner as in the synthesis method of 1-1 using Inter-80 (5.6 g, 20 mmol), Sub1-93(14 g, 22 mmol), Pd₂(dba)₃ (0.9 g, 1 mmol), P(t-Bu)₃(0.4 g, 2 mmol) and NaOt-Bu (5.8 g, 60 mmol) to obtain 14.3 g (yield: 81%) of product.

### 5. Synthesis Example of 1-95

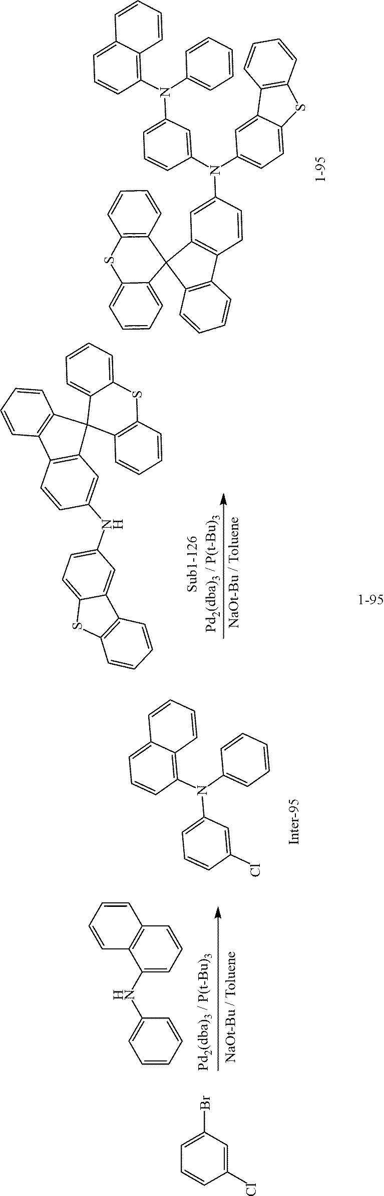

### Synthesis Example of Inter-95

The reaction was carried out in the same manner as in the synthesis method of Inter-1 using 1-bromo-3-chlorobenzene(7.6 g, 40 mmol), N-phenylnaphthalen-1-amine(9.7 g, 44 mmol), Pd₂(dba)₃ (1.8 g, 2 mmol), P(t-Bu)₃(0.8 g, 4 mmol) and NaOt-Bu (11.5 g, 120 mmol) to obtain 11.2 g (yield: 85%) of product.

### Synthesis Example of 1-95

The reaction was carried out in the same manner as in the synthesis method of 1-1 using Inter-95 (6.6 g, 20 mmol), Sub1-126(12 g, 22 mmol), Pd₂(dba)₃ (0.9 g, 1 mmol), P(t-Bu)₃(0.4 g, 2 mmol) and NaOt-Bu (5.8 g, 60 mmol) to obtain 12 g (yield: 72%) of product.

### 6. Synthesis Example of 1-113

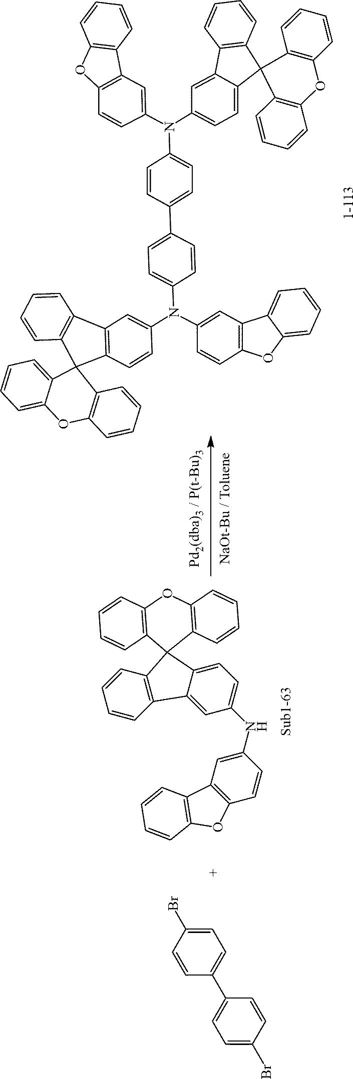

The reaction was carried out in the same manner as in the synthesis method of 1-1 using 4,4′-dibromo-1,1′-biphenyl (3.1 g, 10 mmol), Sub1-63(11.3 g,22 mmol), Pd₂(dba)₃ (0.5 g, 0.5 mmol), P(t-Bu)₃(0.2 g, 1 mmol) and NaOt-Bu (2.9 g, 30 mmol) to obtain 12 g (yield: 72%) of product.

### 7. Synthesis Example of 1-133

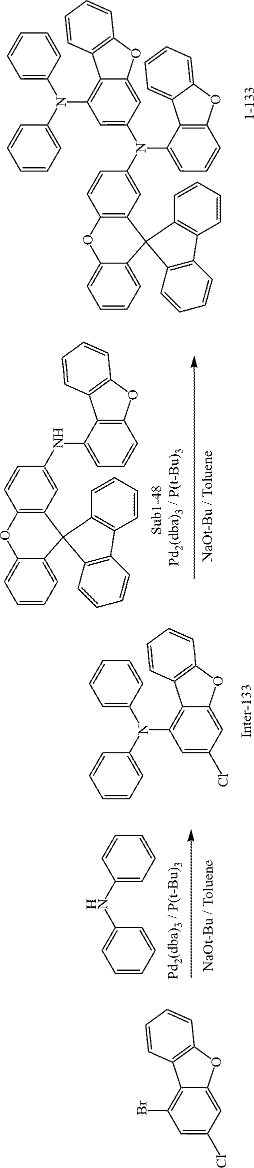

### Synthesis Example of Inter-133

The reaction was carried out in the same manner as in the synthesis method of Inter-1 using 1-bromo-3-chlorodibenzo[b,d]furan(11.3 g, 40 mmol), diphenylamine(7.4 g, 44 mmol), Pd₂(dba)₃ (1.8 g, 2 mmol), P(t-Bu)₃(0.8 g, 4 mmol) and NaOt-Bu (11.5 g, 120 mmol) to obtain 11.7 g (yield: 79%) of product.

### Synthesis Example of 1-133

The reaction was carried out in the same manner as in the synthesis method of 1-1 using Inter-133 (7.4 g, 20 mmol), Sub1-48(12 g, 22 mmol), Pd₂(dba)₃ (0.9 g, 1 mmol), P(t-Bu)₃(0.4 g, 2 mmol) and NaOt-Bu (5.8 g, 60 mmol) to obtain 13.9 g (yield: 82%) of product.

The FD-MS values of the compounds 1-1 to 1-180 of the present invention prepared according to the above synthesis examples are shown in Table 2 below.

TABLE 2

| Compound | FD-MS | Compound | FD-MS |
|---|---|---|---|
| 1-1 | m/z=756.28(C₅₅H₃₆N₂O₂=756.91) | 1-2 | m/z=788.23 (C₅₅H₃₆N₂S₂=789.03) |
| 1-3 | m/z=756.28(C₅₅H₃₆N₂O₂=756.91) | 1-4 | m/z=772.25(C₅₅H₃₆N₂OS=772.97) |
| 1-5 | m/z=756.28(C₅₅H₃₆N₂O₂=756.91) | 1-6 | m/z=772.25(C₅₅H₃₆N₂OS=772.97) |
| 1-7 | m/z=772.25(C₅₅H₃₆N₂OS=772.97) | 1-8 | m/z=772.25(C₅₅H₃₆N₂OS=772.97) |
| 1-9 | m/z=756.28(C₅₅H₃₆N₂O₂=756.91) | 1-10 | m/z=772.25(C₅₅H₃₆N₂OS=772.97) |
| 1-11 | m/z=756.28(C₅₅H₃₆N₂O₂=756.91) | 1-12 | m/z=788.23 (C₅₅H₃₆N₂S₂=789.03) |
| 1-13 | m/z=756.28(C₅₅H₃₆N₂O₂=756.91) | 1-14 | m/z=756.28(C₅₅H₃₆N₂O₂=756.91) |
| 1-15 | m/z=756.28(C₅₅H₃₆N₂O₂=756.91) | 1-16 | m/z=788.23 (C₅₅H₃₆N₂S₂=789.03) |
| 1-17 | m/z=908.34(C₆₇H₄₄N2O₂=909.1) | 1-18 | mlz=974.33(C₇₁H₄₆N₂OS=975.22) |
| 1-19 | m/z=924.32(C₆₇H₄₄N₂OS=925.16) | 1-20 | m/z=1000.35(C₇₃H₄₈N₂OS=1001.26) |
| 1-21 | m/z=832.31(C₆₁H₄N₂O=833) | 1-22 | m/z=848.29(C₆₁H₄₀N₂OS=849.06) |
| 1-23 | m/z=832.31(CₛᵢH₄₀N₂O₂=833) | 1-24 | m/z=914.28(C₆₅H₄₂N₂S₂=915.19) |
| 1-25 | m/z=908.34(C₆₇H₄₄N₂O₂=909.1) | 1-26 | m/z=940.29(C₆₇H₄₄N₂S₂=941.22) |
| 1-27 | m/z=856.31(C₆₃H₄₀N₂O₂=857.03) | 1-28 | m/z=862.27(C₆₁H₃₈N₂O₂S=863.05) |
| 1-29 | mlz=846.29(C₆₁H₃₈N₂O₃=846.99) | 1-30 | m/z=862.27(C₆₁H₃₈N₂O₂S=863.05) |
| 1-31 | m/z=862.27(C₆₁H₃₈N₂O₂S=863.05) | 1-32 | m/z=878.24(C₆₁H₃₈N₂OS₂=879.11) |
| 1-33 | m/z=784.3 1 (C₃₇H₄₀N₂O₂=784.96) | 1-34 | m/z=846.31(C₅₉H₄₃FN₂OS=847.06) |
| 1-35 | m/z=832.31(C₆₁H₄₀N₂O₂=833) | 1-36 | m/z=789.23 (C₃₄H₃₅N₃ S₂=790.02) |
| 1-37 | m/z=1014.36(C₇₃H₅₀N₂O₂Si=1015.3) | 1-38 | m/z=862.28(C₆₁H₃₈N₂O₄=862.99) |
| 1-39 | m/z=872.34(C₆₄H₄₄N₂O₂=873.07) | 1-40 | m/z=953.29(C₆₇H₄₃N₃S₂=954.22) |
| 1-41 | m/z=989.4(C₇₃H₄₃D₅N₂O₂=990.23) | 1-42 | m/z=1040.3(C₇₃H₄₄N₄S₂=1041.3) |
| 1-43 | mlz=934.38(C₆₇H₃₄D₁₀N₂OS=935.22) | 1-44 | m/z=1052.31(C₇₃H₄ₛF₂N₂S₂=1053.3) |
| 1-45 | m/z=898.3(C₆₅H₄₂N₂OS=899.12) | 1-46 | m/z=898.3(C₆₅H₄₂N₂OS=899.12) |
| 1-47 | m/z=958.36(C₇₁H₄₆N₂O₂=959.16) | 1-48 | m/z=1008.37(C₇₅H₄₈N₂O₂=1009.22) |
| 1-49 | m/z=756.28(C₅₅H₃₆N₂O₂=756.91) | 1-50 | m/z=772.25(C₅₅H₃₆N₂OS=772.97) |
| 1-51 | m/z=772.25(C₅₅H₃₆N₂OS=772.97) | 1-52 | m/z=756.28(C₅₅H₃₆N₂O₂=756.91) |
| 1-53 | m/z=756.28(C₅₅H₃₆N₂O₂=756.91) | 1-54 | m/z=772.25(C₅₅H₃₆N₂OS=772.97) |
| 1-55 | m/z=772.25(C₅₅H₃₆N₂OS=772.97) | 1-56 | m/z=788.23 (C₅₅H₃₆N₂S₂=789.03) |
| 1-57 | m/z=772.25(C₅₅H₃₆N₂OS=772.97) | 1-58 | m/z=756.28(C₅₅H₃₆N₂O₂=756.91) |
| 1-59 | m/z=772.25(C₅₅H₃₆N₂OS=772.97) | 1-60 | m/z=772.25(C₅₅H₃₆N₂OS=772.97) |
| 1-61 | m/z=772.25(C₅₅H₃₆N₂OS=772.97) | 1-62 | m/z=788.23 (C₅₅H₃₆N₂S₂=789.03) |
| 1-63 | m/z=756.28(C₅₅H₃₆N₂O₂=756.91) | 1-64 | m/z=756.28(C₅₅H₃₆N₂O₂=756.91) |
| 1-65 | m/z=756.28(C₅₅H₃₆N₂O₂=756.91) | 1-66 | m/z=788.23 (C₅₅H₃₆N₂S₂=789.03) |
| 1-67 | m/z=772.25(C₅₅H₃₆N₂OS=772.97) | 1-68 | m/z=772.25(C₅₅H₃₆N₂OS=772.97) |
| 1-69 | m/z=772.25(C₅₅H₃₆N₂OS=772.97) | 1-70 | m/z=756.28(C₅₅H₃₆N₂O₂=756.91) |
| 1-71 | m/z=756.28(C₅₅H₃₆N₂O₂=756.91) | 1-72 | m/z=772.25(C₅₅H₃₆N₂OS=772.97) |
| 1-73 | m/z=882.32(C₆₅H₄₂N₂O₂=883.06) | 1-74 | m/z=806.29(C₅₉H₃₈N₂O₂=806.97) |
| 1-75 | m/z=882.32(C₆₅H₄₂N₂O₂=883.06) | 1-76 | mlz=974.33(C₇₁H₄₆N₂OS=975.22) |
| 1-77 | m/z=848.29(C₆₁H₄₀N₂OS=849.06) | 1-78 | m/z=832.31(CₛᵢH₄₀N₂O₂=833) |
| 1-79 | m/z=848.29(C₆₁H₄₀N₂OS=849.06) | 1-80 | m/z=882.32(C₆₅H₄₂N₂O₂=883.06) |
| 1-81 | mlz=846.29(C₆₁H₃₈N₂O₃=846.99) | 1-82 | m/z=1013.34(C₇₃H₄₇N₃OS=1014.26) |
| 1-83 | m/z=921.34(C₆₇H₄₃N₃O₂=922.1) | 1-84 | m/z=862.27(C₆₁H₃₈N₂O₂S=863.05) |
| 1-85 | m/z=888.32(C₆₄H₄₄N₂OS=889.13) | 1-86 | wz=912.28(C₆₅H₄₀N₂O₂S=913.11) |
| 1-87 | mlz=846.29(C₆₁H₃₈N₂O₃=846.99) | 1-88 | m/z=912.28(C₆₅H₄₀N₂O₂S=913.11) |
| 1-89 | mlz=832.33(C₅₉H₂₈D₁₀N₂OS=833.09) | 1-90 | m/z=848.29(C₆₁H₄₀N₂OS=849.06) |
| 1-91 | m/z=773.25(C₅₄H₃₅N₃OS=773.95) | 1-92 | m/z=832.31(C₆₁H₄₀N₂O₂=833) |
| 1-93 | m/z=882.32(C₆₅H₄₂N₂O₂=883.06) | 1-94 | m/z=856.31(C₆₃H₄₀N₂O₂=857.03) |
| 1-95 | m/z=838.25 (C₅₉H₃₈N₂S₂=839.09) | 1-96 | m/z=854.33(C₆₁H₄₆N₂OS=855.11) |
| 1-97 | m/z=872.34(C₆₄H₄₄N₂O₂=873.07) | 1-98 | m/z=872.34(C₆₄H₄₄N₂O₂=873.07) |
| 1-99 | m/z=888.32(C₆₄H₄₄N₂OS=889.13) | 1-100 | m/z=824.21 (C₅₅H₃₄F₂N₂S₂=825.01) |
| 1-101 | m/z=822.27(C₅₉H₃₈N₂OS=823.03) | 1-102 | m/z=756.28(C₅₅H₃₆N₂O₂=756.91) |
| 1-103 | m/z=756.28(C₅₅H₃₆N₂O₂=756.91) | 1-104 | m/z=772.25(C₅₅H₃₆N₂OS=772.97) |
| 1-105 | m/z=772.25(C₅₅H₃₆N₂OS=772.97) | 1-106 | m/z=772.25(C₅₅H₃₆N₂OS=772.97) |
| 1-107 | m/z=756.28(C₅₅H₃₆N₂O₂=756.91) | 1-108 | m/z=772.25(C₅₅H₃₆N₂OS=772.97) |
| 1-109 | m/z=806.29(C₅₉H₃₈N₂O₂=806.97) | 1-110 | m/z=914.28(C₆₅H₄₂N₂S₂=915.19) |
| 1-111 | m/z=832.31(C₆₁H₄₀N₂O₂=833) | 1-112 | m/z=1030.31(C₇₃H₄₆N₂OS₂=1031.3) |
| 1-113 | m/z=1176.39(C₈₆H₅₂N₂O₄=1177.37) | 1-114 | m/z=832.31(C₆₁H₄₀N₂O₂=833) |
| 1-115 | m/z=849.28(C₆₀H₃₉N₃OS=850.05) | 1-116 | m/z=924.32(C₆₇H₄₄N₂OS=925.16) |
| 1-117 | m/z=848.29(C₆₁H₄₀N₂OS=849.06) | 1-118 | m/z=832.31(C₆₁H₄₀N₂O₂=833) |
| 1-119 | m/z=908.34(C₆₇H₄₄N₂O₂=909.1) | 1-120 | m/z=908.34(C₆₇H₄₄N₂O₂=909.1) |
| 1-121 | m/z=849.29(C₆₁H₄₀N₂OS=849.06) | 1-122 | m/z=882.32(C₆₅H₄₂N₂O₂=883.06) |
| 1-123 | m/z=838.25(C₅₉H₃₈N₂S₂=839.09) | 1-124 | m/z=832.31(C₆₁H₄₀N₂O₂=833) |
| 1-125 | m/z=898.3(C₆₅H₄₂N₂OS=899.12) | 1-126 | m/z=848.29(C₆₁H₄₀N₂OS=849.06) |
| 1-127 | m/z=806.29(C₅₉H₃₈N₂O₂=806.97) | 1-128 | m/z=822.27(C₅₉H₃₈N₂OS=823.03) |
| 1-129 | m/z=832.31(C₆₁H₄₀N₂O₂=833) | 1-130 | m/z=822.27(C₅₉H₃₈N₂OS=823.03) |
| 1-131 | m/z=806.29(C₅₉H₃₈N₂O₂=806.97) | 1-132 | m/z=832.31(C₆₁H₄₀N₂O₂=833) |
| 1-133 | mlz=846.29(C₆₁H₃₈N₂O₃=846.99) | 1-134 | mlz=846.29(C₆₁H₃₈N₂O₃=846.99) |
| 1-135 | m/z=846.29(C₆₁H₃₈N₂O₃=846.99) | 1-136 | m/z=846.29(C₆₁H₃₃N₂O₃=846.99) |
| 1-137 | m/z=922.32(C₆₇H₄₂N₂O₃=923.08) | 1-138 | mlz=846.29(C₆₁H₃₈N₂O₃=846.99) |
| 1-139 | mlz=846.29(C₆₁H₃₈N₂O₃=846.99) | 1-140 | m/z=862.27(C₆₁H₃₈N₂O₂S=863.05) |
| 1-141 | mlz=846.29(C₆₁H₃₈N₂O₃=846.99) | 1-142 | mlz=846.29(C₆₁H₃₈N₂O₃=846.99) |
| 1-143 | m/z=896.3(C₆₅H₄₀N₂O₃=897.05) | 1-144 | m/z=862.27(C₆₁H₃₈N₂O₂S=863.05) |
| 1-145 | m/z=912.28(C₆₅H₄₀N₂O₂S=913.11) | 1-146 | m/z=862.27(C₆₁H₃₈N₂O₂S=863.05) |
| 1-147 | m/z=862.27(C₆₁H₃₈N₂O₂S=863.05) | 1-148 | m/z=862.27(C₆₁H₃₈N₂O₂S=863.05) |
| 1-149 | m/z=846.29(C₆₁H₃₈N₂O₃=846.99) | 1-150 | mlz=846.29(C₆₁H₃₈N₂O₃=846.99) |
| 1-151 | mlz=846.29(C₆₁H₃₈N₂O₃=846.99) | 1-152 | m/z=862.27(C₆₁H₃₈N₂O₂S=863.05) |
| 1-153 | mlz=846.29(C₆₁H₃₈N₂O₃=846.99) | 1-154 | m/z=862.27(C₆₁H₃₈N₂O₂S=863.05) |
| 1-155 | m/z=846.29(C₆₁H₃₈N₂O₃=846.99) | 1-156 | m/z=894.22(C₆₁H₃₈N₂S₃=895.17) |
| 1-157 | m/z=938.3(C₆₇H₄₂N₂O₂S=939.15) | 1-158 | m/z=846.29(C₆₁H₃₈N₂O₃=846.99) |
| 1-159 | m/z=952.28(Cₛ₇H₄₀N₂O₃S=953.13) | 1-160 | m/z=862.27(C₆₁H₃₈N₂O₂S=863.05) |
| 1-161 | m/z=862.27(C₆₁H₃₈N₂O₂S=863.05) | 1-162 | m/z=846.29(C₆₁H₃₈N₂O₃=846.99) |
| 1-163 | m/z=846.29(C₆₁H₃₈N₂O₃=846.99) | 1-164 | m/z=862.27(C₆₁H₃₈N₂O₂S=863.05) |
| 1-165 | m/z=938.3(C₆₇H₄₂N₂O₂S=939.15) | 1-166 | m/z=922.32(C₆₇H₄₂N₂O₃=923.08) |
| 1-167 | m/z=922.32(C₆₇H₄₂N₂O₃=923.08) | 1-168 | m/z=938.3(C₆₇H₄₂N₂O₂S=939.15) |
| 1-169 | m/z=922.32(C₆₇H₄₂N₂O₃=923.08) | 1-170 | m/z=938.3(C₆₇H₄₂N₂O₂S=939.15) |
| 1-171 | m/z=922.32(C₆₇H₄₂N₂O₃=923.08) | 1-172 | m/z=922.32(C₆₇H₄₂N₂O₃=923.08) |
| 1-173 | m/z=958.36(C₇₁H₄₆N₂O₂=959.16) | 1-174 | m/z=1014.33(C₇₃H₄₆N₂O₂S=1015.24 ) |
| 1-175 | m/z=848.29(C₆₁H₄₀N₂OS=849.06) | 1-176 | m/z=938.3(C₆₇H₄₂N₂O₂S=939.15) |
| 1-177 | m/z=1103.37(C₇₉H₄₉N₃O₄=1104.28) | 1-178 | m/z=1090.37(C₇₈H₅₀N₄OS=1091.35) |
| 1-179 | m/z=948.37(C₇₀H₄₈N₂O₂=949.17) | 1-180 | m/z=1192.37(C₈₆H₅₂N₂O₃S=1193.43 ) |

### Fabrication and Evaluation of Organic Electric Element

### [Test Example 1] Green Organic Electroluminescent Element (An Emission-Auxiliary Layer)

After vacuum-depositing 4,4′,4″-tris[2-naphthyl(phenyl)amino]triphenylamine (hereinafter abbreviated as “2-TNATA”) on an ITO layer (anode) formed on a glass substrate to form a hole injection layer of 60 nm thickness, a hole transport layer of 60 nm thickness was formed by vacuum-depositing N,N′-bis(1-naphthalenyl)-N,N′-bis-phenyl-(1,1′-biphenyl)-4,4′-diamine (hereinafter abbreviated as “NPB”) on the hole injection layer.

Subsequently, an emission-auxiliary layer of 20 nm thickness was formed by vacuum-depositing the compound 1-1 of the present invention on the hole transport layer and 4,4′-N,N′-dicarbazole-biphenyl(hereinafter abbreviated as “CBP”) as a host material and tris(2-phenylpyridine)-iridium (hereinafter abbreviated as “Ir(ppy)₃) as a dopant material in a weight ratio of 95:5 were deposited on the emission-auxiliary layer to form a light emitting layer of 30 nm thickness.

Next, (1,1′-bisphenyl-4-olato)bis(2-methyl-8-quinolinolato)aluminum (hereinafter abbreviated as “BAlq″) was vacuum-deposited to form a hole blocking layer of 10 nm thickness on the light emitting layer, and tris-(8-hydroxyquinoline)aluminum (hereinafter abbreviated as “Alq₃”) was vacuum-deposited to form an electron transport layer of 40 nm thickness on the hole blocking layer.

Thereafter, LiF was deposited to form an electron injection layer of 0.2 nm thickness, and then Al was deposited to form a cathode of 150 nm thickness.

### [Test Example 2] to [Test Example 20]

The organic electroluminescent elements were fabricated in the same manner as described in Example 1 except that the compounds of the present invention described in the following Table 3, instead of compound 1-1 of the present invention, were used as material of an emission-auxiliary layer.

### [Comparative Example 1]

The organic electroluminescent element was fabricated in the same manner as in Example 1, except that an emission-auxiliary layer was not formed.

### [Comparative Example 2] to [Comparative Example 4]

The organic electroluminescent elements were fabricated in the same manner as described in Example 1 except that one of Comparative Compounds A to C, instead of compound 1-1 of the present invention, was used as material of an emission-auxiliary layer.

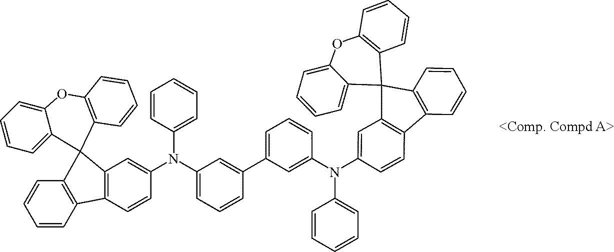 [SMILES: c1ccc(N(c2cccc(-c3cccc(N(c4ccccc4)c4ccc5c(c4)C4(c6ccccc6Oc6ccccc64)c4ccccc4-5)c3)c2)c2ccc3c(c2)C2(c4ccccc4Oc4ccccc42)c2ccccc2-3)cc1]

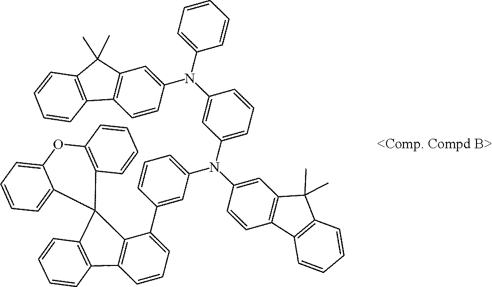 [SMILES: CC1(C)c2ccccc2-c2ccc(N(c3ccccc3)c3cccc(N(c4cccc(-c5cccc6c5C5(c7ccccc7Oc7ccccc75)c5ccccc5-6)c4)c4ccc5c(c4)C(C)(C)c4ccccc4-5)c3)cc21]

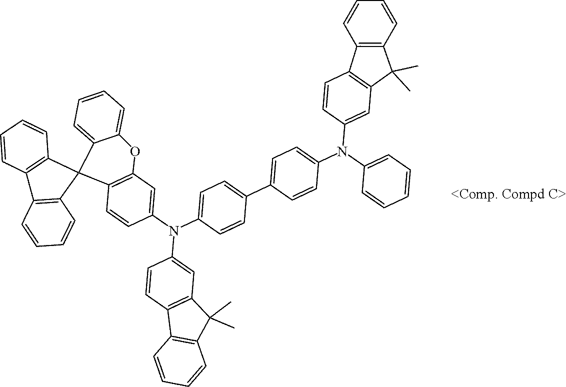 [SMILES: CC1(C)c2ccccc2-c2ccc(N(c3ccccc3)c3ccc(-c4ccc(N(c5ccc6c(c5)Oc5ccccc5C65c6ccccc6-c6ccccc65)c5ccc6c(c5)C(C)(C)c5ccccc5-6)cc4)cc3)cc21]

Electroluminescence (EL) characteristics were measured with PR-650 (Photoresearch) by applying a forward bias DC voltage to the organic electroluminescent elements prepared in Examples 1 to 20 of the present invention and Comparative Examples 1 to 4. T95 life time was measured using a life time measuring apparatus manufactured by mc science Inc. at reference brightness of 5000 cd/m². The measurement results are shown in Tables 3 below.

TABLE 3

| | Compound | Voltage (V) | Current Density (mA/cm²) | Brightness (cd/m²) | Efficiency (cd/A) | Lifetime T(95) | CIE | |
|---|---|---|---|---|---|---|---|---|
| | | | | | | | x | y |
| comp.Ex(1) | - | 6 | 21.5 | 5000 | 23.3 | 57.3 | 0.32 | 0.61 |
| comp.Ex(2) | Comp. compd 1 | 5.7 | 15.9 | 5000 | 31.4 | 65.4 | 0.32 | 0.60 |
| comp.Ex(3) | Comp. compd 2 | 5.9 | 17.0 | 5000 | 29.4 | 48.2 | 0.32 | 0.62 |
| comp.Ex(4) | Comp. compd 3 | 5.6 | 14.1 | 5000 | 35.4 | 76.3 | 0.32 | 0.60 |
| Test Ex.(1) | 1-1 | 5.4 | 8.6 | 5000 | 58.0 | 102.6 | 0.33 | 0.62 |
| Test Ex.(2) | 1-14 | 5.3 | 9.9 | 5000 | 50.4 | 109.6 | 0.33 | 0.61 |
| Test Ex.(3) | 1-15 | 5.2 | 10.8 | 5000 | 46.3 | 105.9 | 0.32 | 0.61 |
| Test Ex.(4) | 1-35 | 5.4 | 8.7 | 5000 | 57.4 | 91.1 | 0.32 | 0.60 |
| Test Ex.(5) | 1-39 | 5.2 | 10.7 | 5000 | 46.8 | 100.1 | 0.32 | 0.62 |
| Test Ex.(6) | 1-49 | 5.2 | 9.3 | 5000 | 53.5 | 102.5 | 0.33 | 0.63 |
| Test Ex.(7) | 1-53 | 5.3 | 10.3 | 5000 | 48.7 | 106.7 | 0.32 | 0.62 |
| Test Ex.(8) | 1-60 | 5.2 | 9.3 | 5000 | 53.6 | 87.3 | 0.32 | 0.62 |
| Test Ex.(9) | 1-81 | 5.2 | 9.5 | 5000 | 52.7 | 104.2 | 0.33 | 0.62 |
| Test Ex.(10) | 1-84 | 5.2 | 10.6 | 5000 | 47.1 | 85.2 | 0.33 | 0.63 |
| Test Ex.(11) | 1-112 | 5.2 | 10.1 | 5000 | 49.4 | 88.7 | 0.33 | 0.62 |
| Test Ex.(12) | 1-124 | 5.3 | 10.7 | 5000 | 46.9 | 99.6 | 0.32 | 0.62 |
| Test Ex.(13) | 1-131 | 5.1 | 11.2 | 5000 | 44.4 | 93.7 | 0.33 | 0.62 |
| Test Ex.(14) | 1-132 | 5.4 | 8.8 | 5000 | 56.7 | 98.7 | 0.33 | 0.60 |
| Test Ex.(15) | 1-133 | 4.9 | 12.4 | 5000 | 40.5 | 91.8 | 0.33 | 0.63 |
| Test Ex.(16) | 1-136 | 5.0 | 12.5 | 5000 | 40.1 | 90.9 | 0.33 | 0.61 |
| Test Ex.(17) | 1-145 | 5.1 | 11.3 | 5000 | 44.3 | 89.7 | 0.32 | 0.62 |
| Test Ex.(18) | 1-149 | 5.1 | 12.3 | 5000 | 40.8 | 107.0 | 0.33 | 0.61 |
| Test Ex.(19) | 1-157 | 5.0 | 11.3 | 5000 | 44.4 | 86.8 | 0.32 | 0.61 |
| Test Ex.(20) | 1-176 | 5.0 | 10.9 | 5000 | 45.7 | 86.0 | 0.33 | 0.61 |

As can be seen from Table 3, when compound of the present invention is used as material for an emission-auxiliary layer, the driving voltage is significantly lowered, and the luminous efficiency and lifespan are remarkably improved, compared to the Comparative Example 1 not forming an emission-auxiliary layer, or Comparative Examples 2 to 3 using one of Comparative Compound A to Comparative Compound C as material for an emission-auxiliary layer.

Comparative Compounds A to C have similar skeletons to compound of the present invention, but differ in the type of substituent substituted on the amine group.

In the compound of the present invention, a substituent containing Formula A-1 or Formula A-2 is substituted on an amine group and at the same time a heterocycle containing a moiety such as dibenzofuran, dibenzothiophene, or carbazole having high thermal stability and hole characteristics is substituted on the same amine group. In contrast, Comparative Compounds A to C are similar to the present invention in that an amine group is substituted with a substituent represented by Formula A-1 or Formula A-2 (spiro[fluorene-9,9′-xanthene]), but differ in that the remaining substituent substituted on the same amine group is phenyl groups (in the case of Comparative Compound A) or fluorenyl group (in the cases of Comparative Compounds B and C).

Due to this difference in substituents, in the case of using the compound of the present invention compared to the comparative compound, the characteristics of the element are significantly improved. This seems to be because the space in which holes can be trapped becomes relatively larger, as a heterocyclic group (dibenzofuran, dibenzothiophene, carbazole, etc.) having high thermal stability and hole properties is substituted on an amine group, thereby increasing the charge balance in the light emitting layer.

Accordingly, it can be seen that although the structures of the compounds are similar, the physical properties of the compound such as hole characteristics, light efficiency characteristics, hole injection and transport characteristics, etc., vary depending on the type of substituent, and these differences affect the characteristics of the element.

Although the exemplary embodiments of the present invention have been described for illustrative purposes, those skilled in the art to which the present invention pertains will be capable of various modifications without departing from the essential characteristics of the present invention. Therefore, the embodiment disclosed herein is intended to illustrate the present invention rather than to limit the present invention and the scope of the present invention is not limited by the embodiments. The scope of the present invention shall be construed on the basis of the accompanying claims, and it shall be construed that all of the technical ideas included within the scope equivalent to the claims belong to the present invention.

What is claimed is:

1. A compound of Formula 1: 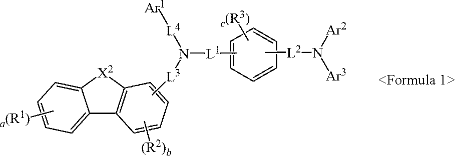
wherein:
Ar¹ is Formula A-1 or Formula A-2, 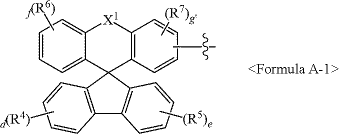 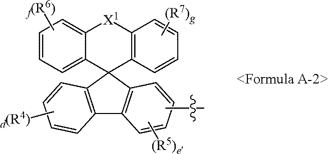
Ar² and Ar³ are each independently selected from the group consisting of a C₆-C₆₀ aryl group, a fluorenyl group, a C₂-C₆₀ heterocyclic group containing at least one heteroatom of O, N, S, Si and P, and a C₃-C₆₀ aliphatic ring group,
X¹ is O or S,
X² is N(R′), O or S,
R¹ to R⁷ are each independently selected from the group consisting of hydrogen, deuterium, halogen, a cyano group, a nitro group, a C₆-C₆₀ aryl group, a fluorenyl group, a C₂-C₆₀ heterocyclic group comprising at least one heteroatom selected from the group consisting of O, N, S, Si and P, a C₃-C₆₀ aliphatic ring group, a C₁-C₃₀ alkyl group, a C₂-C₃₀ alkenyl group, a C₂-C₃₀ alkynyl group, a C₁-C₃₀ alkoxyl group, and a C₆-C₃₀ aryloxy group, and adjacent groups may be bonded to each other to form a ring.
a, c, d, e, f and g are each an integer of 0 to 4, b, e′ and g′ are each an integer of 0 to 3, and where each of these is an integer of 2 or more, each of R¹s, each of R²s, each of R³s, each of R⁴s, each of R⁵s, each of R⁶s, each of R⁷s are the same as or different from each other,
L¹ to L⁴ are each independently selected from the group consisting of a single bond, a C₆-C₆₀ arylene group, a fluorenylene group, a C₃-C₆₀ aliphatic ring group, and a C₂-C₆₀ heterocyclic group containing at least one heteroatom of O, N, S, Si and P,
R′ is selected from the group consisting of a C₆-C₆₀ aryl group, a fluorenyl group, a C₂-C₆₀ heterocyclic group containing at least one heteroatom of O, N, S, Si and P and a C₃-C₆₀ aliphatic ring group, and
the aryl group, the arylene group, the fluorenyl group, the fluorenylene group, the heterocyclic group, the aliphatic ring group, the alkyl group, the alkenyl group, the alkynyl group, the alkoxyl group, the aryloxyl group, and the ring formed by adjacent groups may be each optionally substituted with one or more substituents selected from the group consisting of deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group.

2. The compound of claim 1, wherein Formula 1 is represented by one of Formula 1-1 to Formula 1-6: 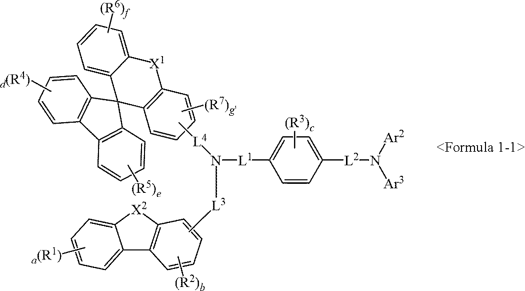 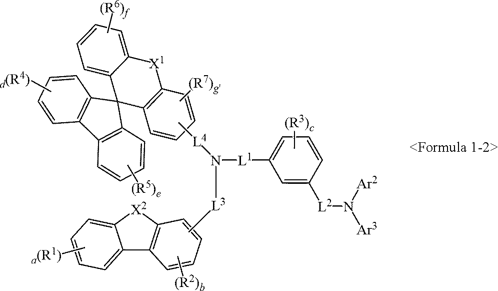 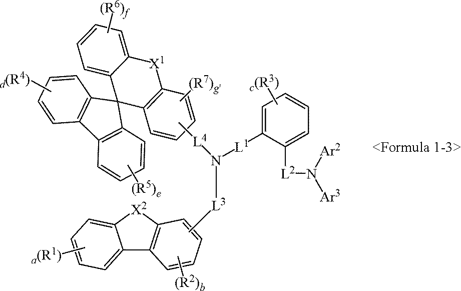 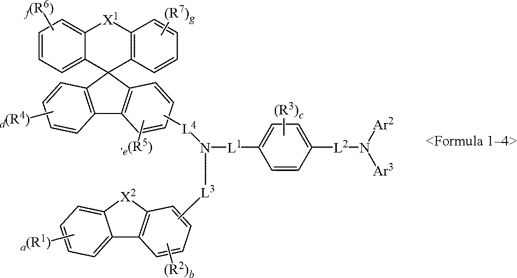 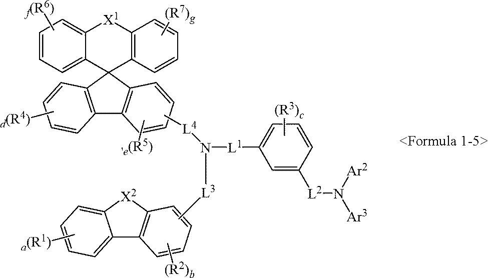 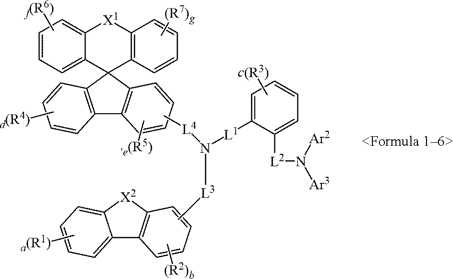
wherein, in Formula 1-1 to Formula 1-6, X¹, X², L¹ to L⁴, Ar², Ar³, R¹ to R⁷, and a to g are the same as defined in claim 1, and e′ and g′ are each an integer of 0 to 3.

3. The compound of claim 1, wherein Formula 1 is represented by one of Formula 1-7 to Formula 1-18: 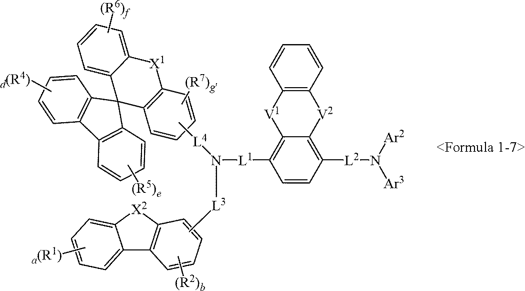 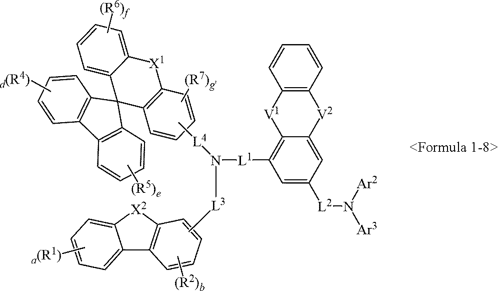 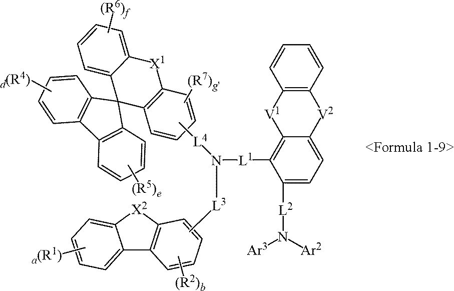 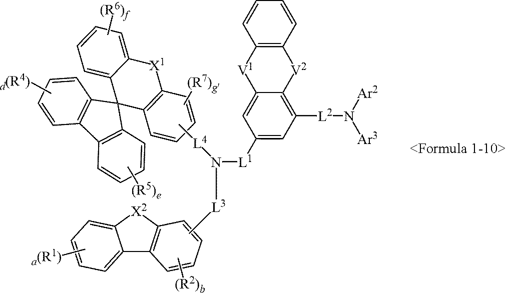 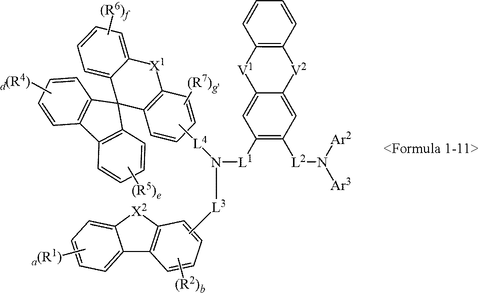 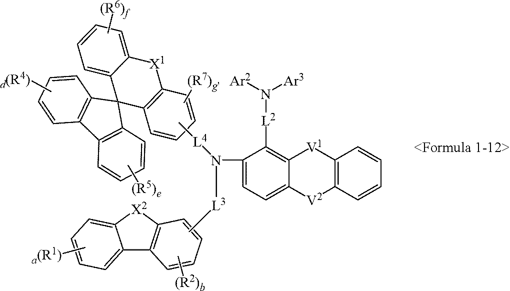 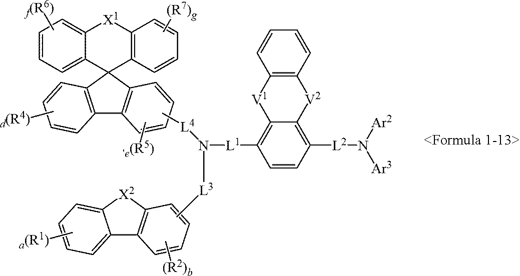 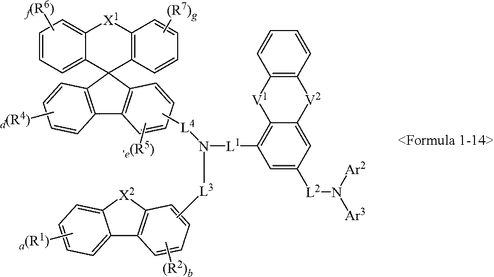 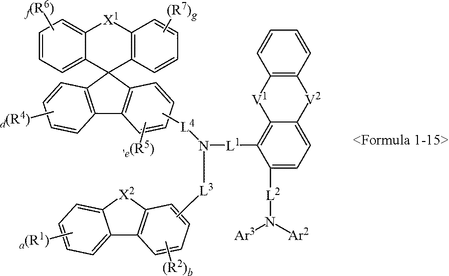 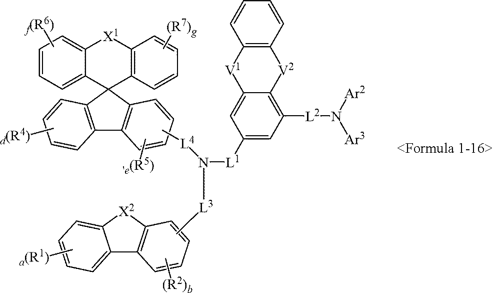 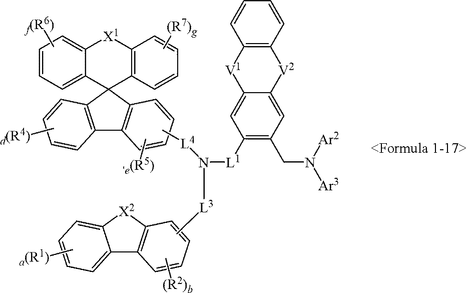 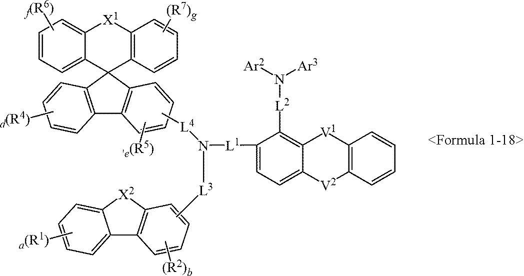
wherein, in Formula 1-7 to Formula 1-18, X¹, X², L¹ to L⁴, Ar², Ar³, R¹, R², R⁴ to R⁷, and a to g are the same as defined in claim 1, e′ and g′ are each an integer of 0 to 3,
V¹ and V² are each independently a single bond, O, S, C(R₁)(R₂) or N(R₃), with a proviso that both V¹ and V² are not a single bond at the same time,
R₁ and R₂ are each independently selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group, and R₁ and R₂ may be linked to each other to form a ring, and
R₃ is selected from the group consisting of a C₆-C₂₀ aryl group, a fluorenyl group, a C₂-C₂₀ heterocyclic group containing at least one heteroatom of O, N, S, Si and P, a C₃-C₂₀ aliphatic ring group, a C₁-C₂₀ alkoxyl group, a C₆-C₂₀ aryloxy group, a C₁-C₂₀ alkyl group, a C₂-C₂₀ alkenyl group, and a C₂-C₂₀ alkynyl group.

4. The compound of claim 1, wherein at least one of Ar² and Ar³ is selected from the group consisting of Formula 2-1 to Formula 2-6: 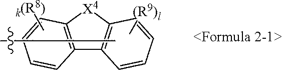 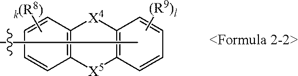 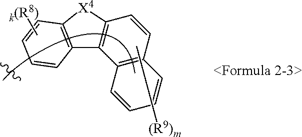 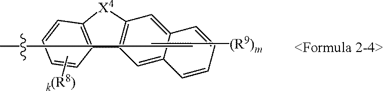 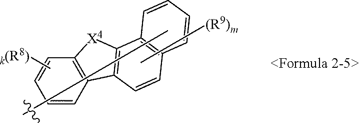 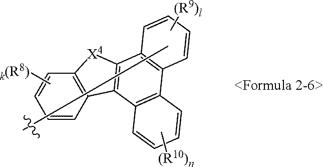
wherein, in Formula 2-1 to Formula 2-6,
X⁴ and X⁵ are each independently O, S, C(R₁)(R₂) or N(R₃),
R₁, R₂, R⁸, R⁹ and R¹⁰ are each independently selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group, adjacent groups may be linked to each other to form a ring, and R₁ and R₂ may be linked to each other to form a ring,
k, 1 and n are each an integer of 0 to 4, m is an integer of 0 to 6, and where each of these is an integer of 2 or more, each of R⁸s, each of R⁹s, each of R¹⁰s are the same as or different from each other, and
R₃ is selected from the group consisting of a C₆-C₂₀ aryl group, a fluorenyl group, a C₂-C₂₀ heterocyclic group containing at least one heteroatom of O, N, S, Si and P, a C₃-C₂₀ aliphatic ring group, a C₁-C₂₀ alkoxyl group, a C₆-C₂₀ aryloxy group, a C₁-C₂₀ alkyl group, a C₂-C₂₀ alkenyl group, and a C₂-C₂₀ alkynyl group.

5. The compound of claim 1, wherein the compound represented by Formula 1 is one of the following compounds: 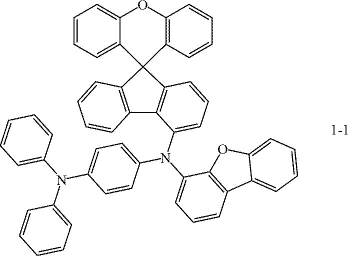 [SMILES: c1ccc(N(c2ccccc2)c2ccc(N(c3cccc4c3-c3ccccc3C43c4ccccc4Oc4ccccc43)c3cccc4c3oc3ccccc34)cc2)cc1] 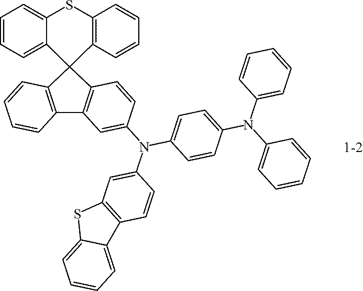 [SMILES: c1ccc(N(c2ccccc2)c2ccc(N(c3ccc4c(c3)-c3ccccc3C43c4ccccc4Sc4ccccc43)c3ccc4c(c3)sc3ccccc34)cc2)cc1] 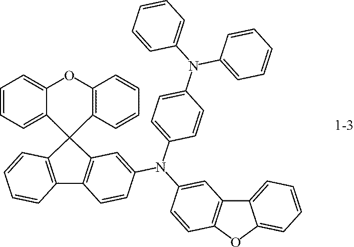 [SMILES: c1ccc(N(c2ccccc2)c2ccc(N(c3ccc4c(c3)C3(c5ccccc5Oc5ccccc53)c3ccccc3-4)c3ccc4oc5ccccc5c4c3)cc2)cc1] 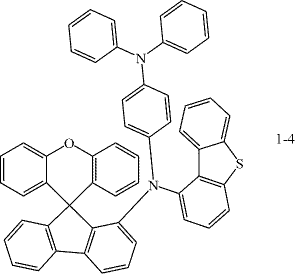 [SMILES: c1ccc(N(c2ccccc2)c2ccc(N(c3cccc4c3C3(c5ccccc5Oc5ccccc53)c3ccccc3-4)c3cccc4sc5ccccc5c34)cc2)cc1] 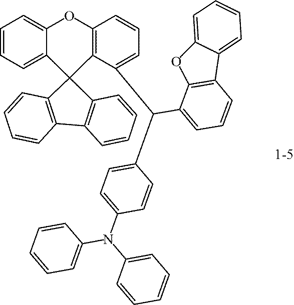 [SMILES: c1ccc(N(c2ccccc2)c2ccc(C(c3cccc4c3C3(c5ccccc5O4)c4ccccc4-c4ccccc43)c3cccc4c3oc3ccccc34)cc2)cc1] 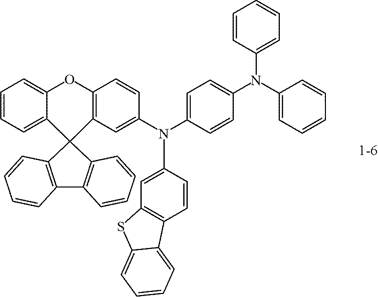 [SMILES: c1ccc(N(c2ccccc2)c2ccc(N(c3ccc4c(c3)C3(c5ccccc5O4)c4ccccc4-c4ccccc43)c3ccc4c(c3)sc3ccccc34)cc2)cc1] 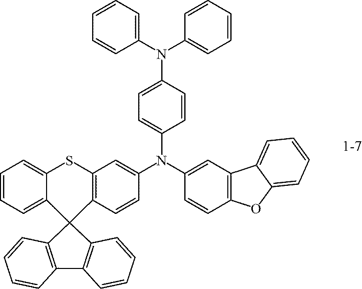 [SMILES: c1ccc(N(c2ccccc2)c2ccc(N(c3ccc4c(c3)Sc3ccccc3C43c4ccccc4-c4ccccc43)c3ccc4oc5ccccc5c4c3)cc2)cc1] 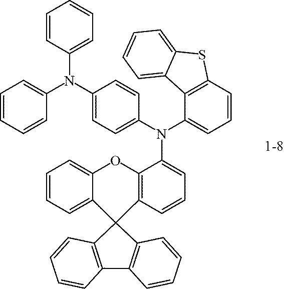 [SMILES: c1ccc(N(c2ccccc2)c2ccc(N(c3cccc4c3Oc3ccccc3C43c4ccccc4-c4ccccc43)c3cccc4sc5ccccc5c34)cc2)cc1] 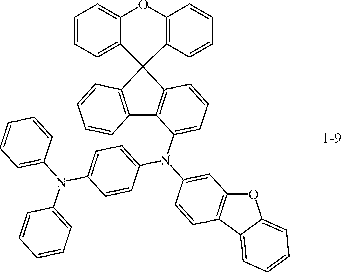 [SMILES: c1ccc(N(c2ccccc2)c2ccc(N(c3ccc4c(c3)oc3ccccc34)c3cccc4c3-c3ccccc3C43c4ccccc4Oc4ccccc43)cc2)cc1] 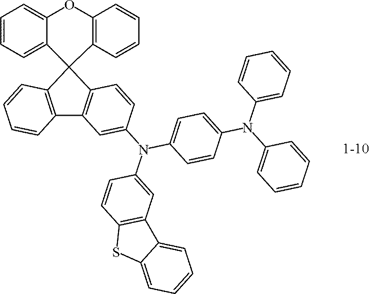 [SMILES: c1ccc(N(c2ccccc2)c2ccc(N(c3ccc4c(c3)-c3ccccc3C43c4ccccc4Oc4ccccc43)c3ccc4sc5ccccc5c4c3)cc2)cc1] 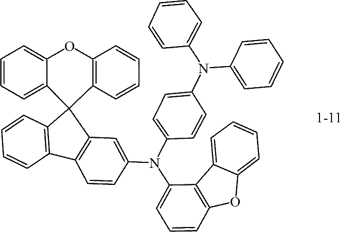 [SMILES: c1ccc(N(c2ccccc2)c2ccc(N(c3ccc4c(c3)C3(c5ccccc5Oc5ccccc53)c3ccccc3-4)c3cccc4oc5ccccc5c34)cc2)cc1] 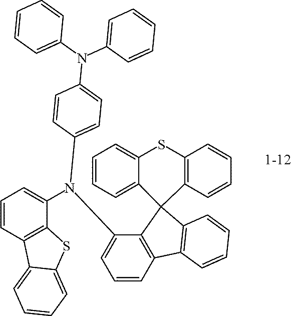 [SMILES: c1ccc(N(c2ccccc2)c2ccc(N(c3cccc4c3C3(c5ccccc5Sc5ccccc53)c3ccccc3-4)c3cccc4c3sc3ccccc34)cc2)cc1] 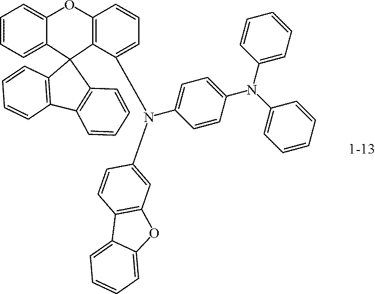 [SMILES: c1ccc(N(c2ccccc2)c2ccc(N(c3ccc4c(c3)oc3ccccc34)c3cccc4c3C3(c5ccccc5O4)c4ccccc4-c4ccccc43)cc2)cc1] 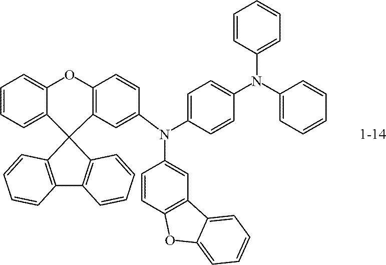 [SMILES: c1ccc(N(c2ccccc2)c2ccc(N(c3ccc4c(c3)C3(c5ccccc5O4)c4ccccc4-c4ccccc43)c3ccc4oc5ccccc5c4c3)cc2)cc1] 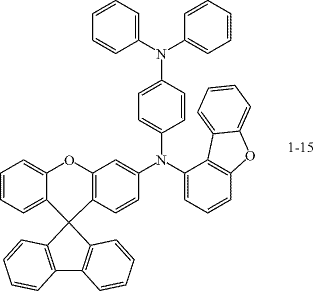 [SMILES: c1ccc(N(c2ccccc2)c2ccc(N(c3ccc4c(c3)Oc3ccccc3C43c4ccccc4-c4ccccc43)c3cccc4oc5ccccc5c34)cc2)cc1] 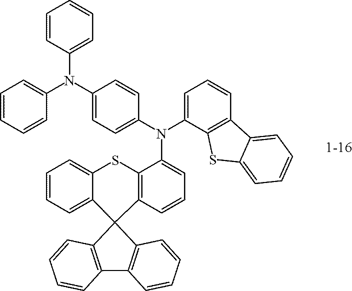 [SMILES: c1ccc(N(c2ccccc2)c2ccc(N(c3cccc4c3Sc3ccccc3C43c4ccccc4-c4ccccc43)c3cccc4c3sc3ccccc34)cc2)cc1] 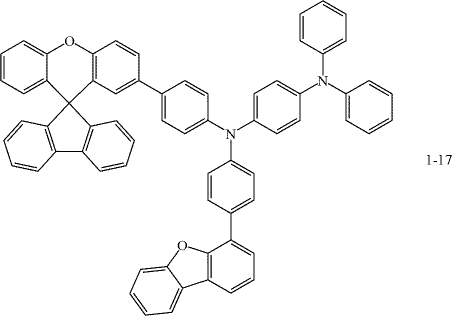 [SMILES: c1ccc(N(c2ccccc2)c2ccc(N(c3ccc(-c4ccc5c(c4)C4(c6ccccc6O5)c5ccccc5-c5ccccc54)cc3)c3ccc(-c4cccc5c4oc4ccccc45)cc3)cc2)cc1] 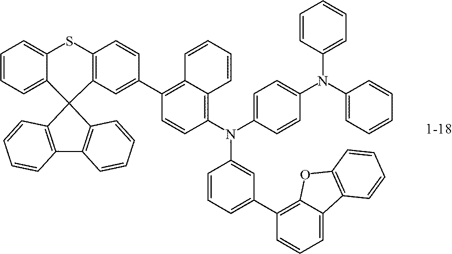 [SMILES: c1ccc(N(c2ccccc2)c2ccc(N(c3cccc(-c4cccc5c4oc4ccccc45)c3)c3ccc(-c4ccc5c(c4)C4(c6ccccc6S5)c5ccccc5-c5ccccc54)c4ccccc34)cc2)cc1] 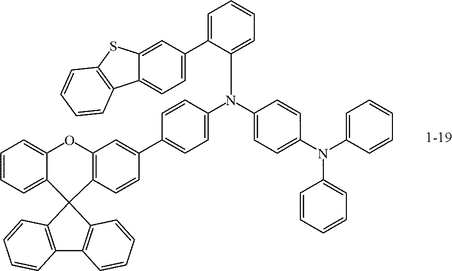 [SMILES: c1ccc(N(c2ccccc2)c2ccc(N(c3ccc(-c4ccc5c(c4)Oc4ccccc4C54c5ccccc5-c5ccccc54)cc3)c3ccccc3-c3ccc4c(c3)sc3ccccc34)cc2)cc1] 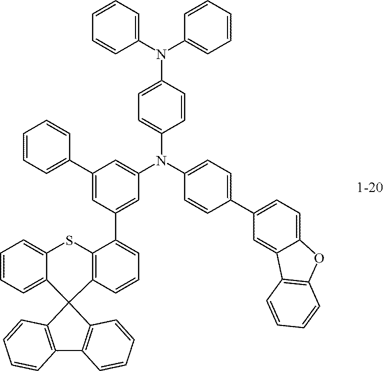 [SMILES: c1ccc(-c2cc(-c3cccc4c3Sc3ccccc3C43c4ccccc4-c4ccccc43)cc(N(c3ccc(-c4ccc5oc6ccccc6c5c4)cc3)c3ccc(N(c4ccccc4)c4ccccc4)cc3)c2)cc1] 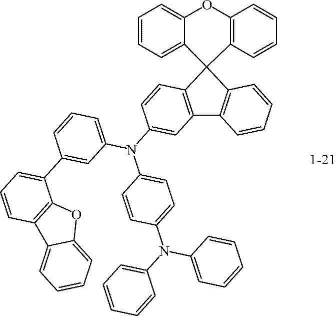 [SMILES: c1ccc(N(c2ccccc2)c2ccc(N(c3cccc(-c4cccc5c4oc4ccccc45)c3)c3ccc4c(c3)-c3ccccc3C43c4ccccc4Oc4ccccc43)cc2)cc1] 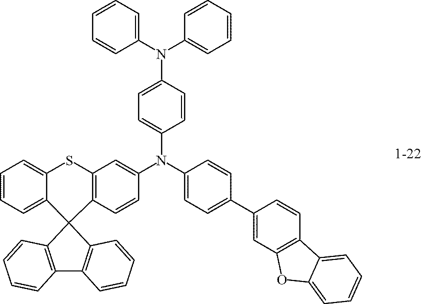 [SMILES: c1ccc(N(c2ccccc2)c2ccc(N(c3ccc(-c4ccc5c(c4)oc4ccccc45)cc3)c3ccc4c(c3)Sc3ccccc3C43c4ccccc4-c4ccccc43)cc2)cc1] 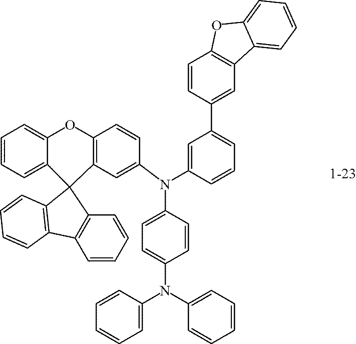 [SMILES: c1ccc(N(c2ccccc2)c2ccc(N(c3cccc(-c4ccc5oc6ccccc6c5c4)c3)c3ccc4c(c3)C3(c5ccccc5O4)c4ccccc4-c4ccccc43)cc2)cc1] 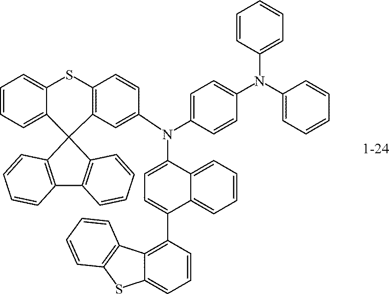 [SMILES: c1ccc(N(c2ccccc2)c2ccc(N(c3ccc4c(c3)C3(c5ccccc5S4)c4ccccc4-c4ccccc43)c3ccc(-c4cccc5sc6ccccc6c45)c4ccccc34)cc2)cc1] 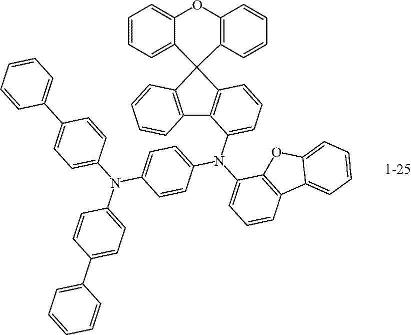 [SMILES: c1ccc(-c2ccc(N(c3ccc(-c4ccccc4)cc3)c3ccc(N(c4cccc5c4-c4ccccc4C54c5ccccc5Oc5ccccc54)c4cccc5c4oc4ccccc45)cc3)cc2)cc1] 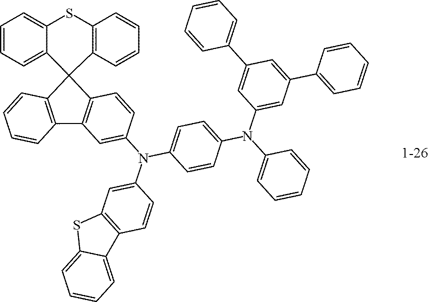 [SMILES: c1ccc(-c2cc(-c3ccccc3)cc(N(c3ccccc3)c3ccc(N(c4ccc5c(c4)-c4ccccc4C54c5ccccc5Sc5ccccc54)c4ccc5c(c4)sc4ccccc45)cc3)c2)cc1] 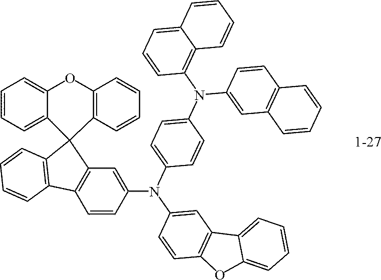 [SMILES: c1ccc2c(c1)Oc1ccccc1C21c2ccccc2-c2ccc(N(c3ccc(N(c4ccc5ccccc5c4)c4cccc5ccccc45)cc3)c3ccc4oc5ccccc5c4c3)cc21] 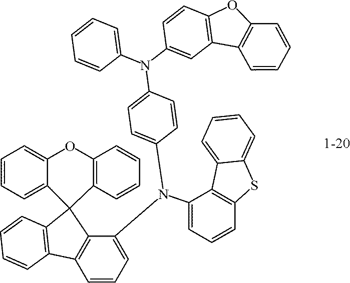 [SMILES: c1ccc(N(c2ccc(N(c3cccc4c3C3(c5ccccc5Oc5ccccc53)c3ccccc3-4)c3cccc4sc5ccccc5c34)cc2)c2ccc3oc4ccccc4c3c2)cc1] 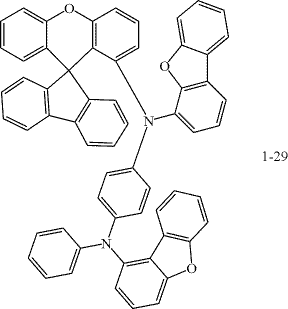 [SMILES: c1ccc(N(c2ccc(N(c3cccc4c3C3(c5ccccc5O4)c4ccccc4-c4ccccc43)c3cccc4c3oc3ccccc34)cc2)c2cccc3oc4ccccc4c23)cc1] 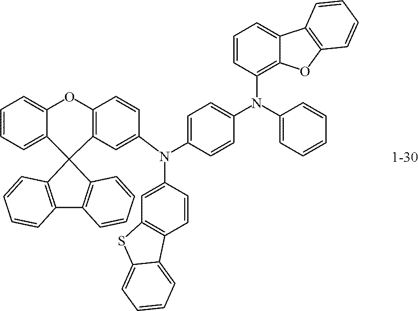 [SMILES: c1ccc(N(c2ccc(N(c3ccc4c(c3)C3(c5ccccc5O4)c4ccccc4-c4ccccc43)c3ccc4c(c3)sc3ccccc34)cc2)c2cccc3c2oc2ccccc23)cc1] 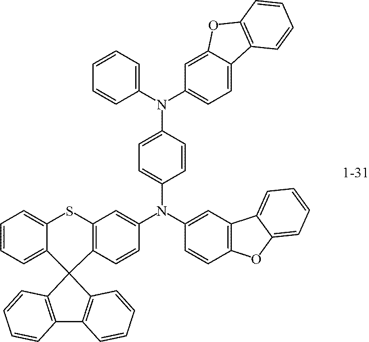 [SMILES: c1ccc(N(c2ccc(N(c3ccc4c(c3)Sc3ccccc3C43c4ccccc4-c4ccccc43)c3ccc4oc5ccccc5c4c3)cc2)c2ccc3c(c2)oc2ccccc23)cc1] 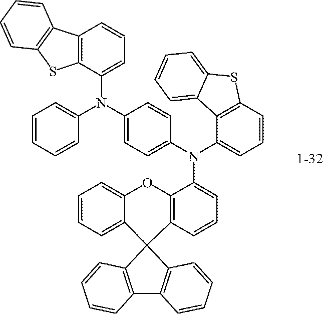 [SMILES: c1ccc(N(c2ccc(N(c3cccc4c3Oc3ccccc3C43c4ccccc4-c4ccccc43)c3cccc4sc5ccccc5c34)cc2)c2cccc3c2sc2ccccc23)cc1] 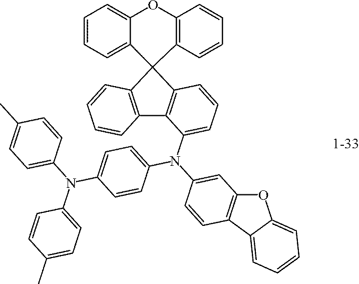 [SMILES: Cc1ccc(N(c2ccc(C)cc2)c2ccc(N(c3ccc4c(c3)oc3ccccc34)c3cccc4c3-c3ccccc3C43c4ccccc4Oc4ccccc43)cc2)cc1] 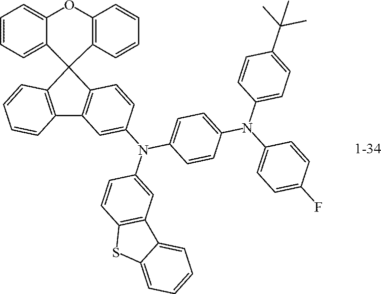 [SMILES: CC(C)(C)c1ccc(N(c2ccc(F)cc2)c2ccc(N(c3ccc4c(c3)-c3ccccc3C43c4ccccc4Oc4ccccc43)c3ccc4sc5ccccc5c4c3)cc2)cc1] 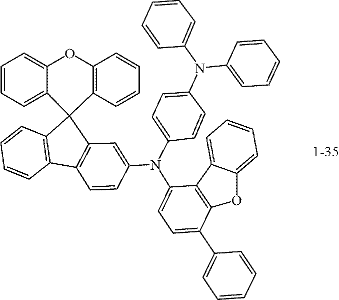 [SMILES: c1ccc(-c2ccc(N(c3ccc(N(c4ccccc4)c4ccccc4)cc3)c3ccc4c(c3)C3(c5ccccc5Oc5ccccc53)c3ccccc3-4)c3c2oc2ccccc23)cc1] 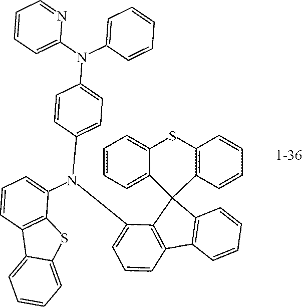 [SMILES: c1ccc(N(c2ccc(N(c3cccc4c3C3(c5ccccc5Sc5ccccc53)c3ccccc3-4)c3cccc4c3sc3ccccc34)cc2)c2ccccn2)cc1] 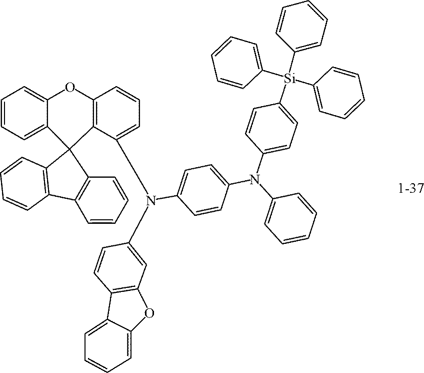 [SMILES: c1ccc(N(c2ccc(N(c3ccc4c(c3)oc3ccccc34)c3cccc4c3C3(c5ccccc5O4)c4ccccc4-c4ccccc43)cc2)c2ccc([Si](c3ccccc3)(c3ccccc3)c3ccccc3)cc2)cc1] 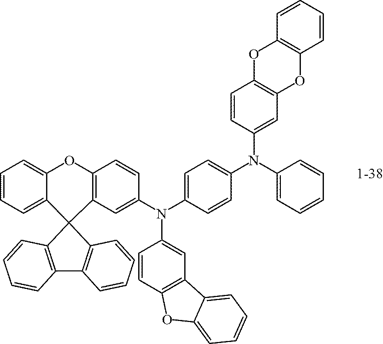 [SMILES: c1ccc(N(c2ccc(N(c3ccc4c(c3)C3(c5ccccc5O4)c4ccccc4-c4ccccc43)c3ccc4oc5ccccc5c4c3)cc2)c2ccc3c(c2)Oc2ccccc2O3)cc1] 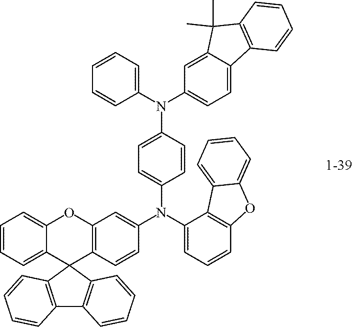 [SMILES: CC1(C)c2ccccc2-c2ccc(N(c3ccccc3)c3ccc(N(c4ccc5c(c4)Oc4ccccc4C54c5ccccc5-c5ccccc54)c4cccc5oc6ccccc6c45)cc3)cc21] 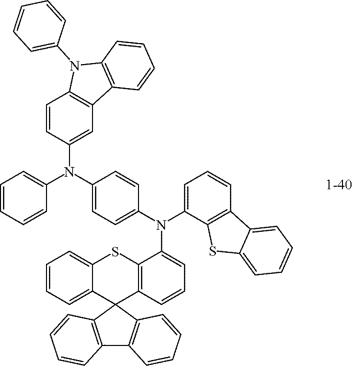 [SMILES: c1ccc(N(c2ccc(N(c3cccc4c3Sc3ccccc3C43c4ccccc4-c4ccccc43)c3cccc4c3sc3ccccc34)cc2)c2ccc3c(c2)c2ccccc2n3-c2ccccc2)cc1] 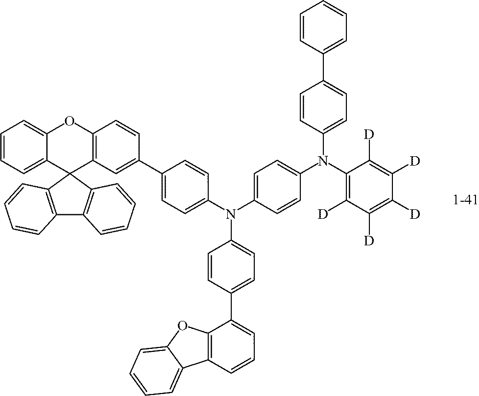 [SMILES: [2H]c1c([2H])c([2H])c(N(c2ccc(-c3ccccc3)cc2)c2ccc(N(c3ccc(-c4ccc5c(c4)C4(c6ccccc6O5)c5ccccc5-c5ccccc54)cc3)c3ccc(-c4cccc5c4oc4ccccc45)cc3)cc2)c([2H])c1[2H]] 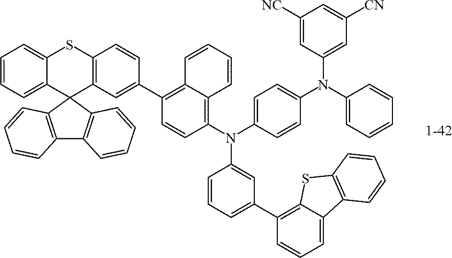 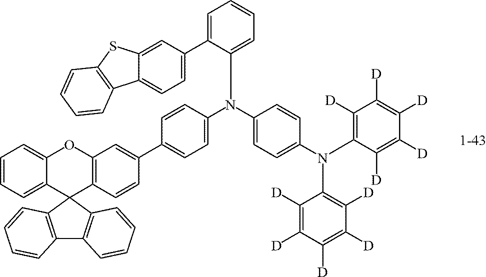 [SMILES: [2H]c1c([2H])c([2H])c(N(c2ccc(N(c3ccc(-c4ccc5c(c4)Oc4ccccc4C54c5ccccc5-c5ccccc54)cc3)c3ccccc3-c3ccc4c(c3)sc3ccccc34)cc2)c2c([2H])c([2H])c([2H])c([2H])c2[2H])c([2H])c1[2H]] 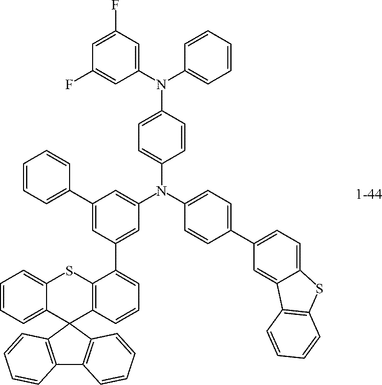 [SMILES: Fc1cc(F)cc(N(c2ccccc2)c2ccc(N(c3ccc(-c4ccc5sc6ccccc6c5c4)cc3)c3cc(-c4ccccc4)cc(-c4cccc5c4Sc4ccccc4C54c5ccccc5-c5ccccc54)c3)cc2)c1] 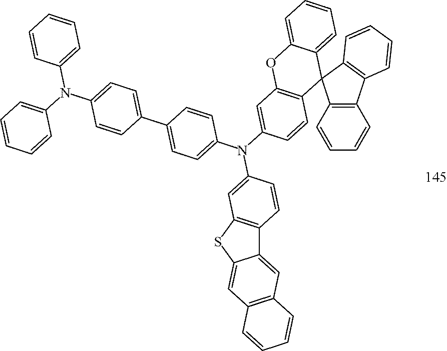 [SMILES: c1ccc(N(c2ccccc2)c2ccc(-c3ccc(N(c4ccc5c(c4)Oc4ccccc4C54c5ccccc5-c5ccccc54)c4ccc5c(c4)sc4cc6ccccc6cc45)cc3)cc2)cc1] 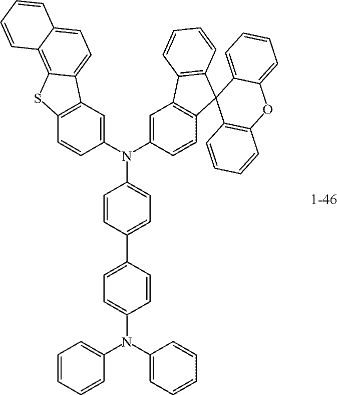 [SMILES: c1ccc(N(c2ccccc2)c2ccc(-c3ccc(N(c4ccc5c(c4)-c4ccccc4C54c5ccccc5Oc5ccccc54)c4ccc5sc6c7ccccc7ccc6c5c4)cc3)cc2)cc1] 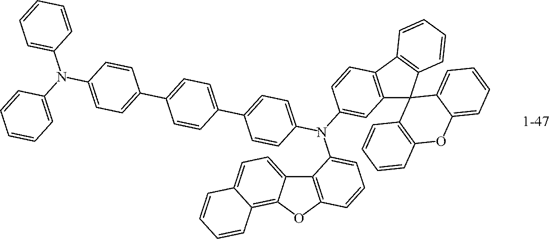 [SMILES: c1ccc(N(c2ccccc2)c2ccc(-c3ccc(-c4ccc(N(c5ccc6c(c5)C5(c7ccccc7Oc7ccccc75)c5ccccc5-6)c5cccc6oc7c8ccccc8ccc7c56)cc4)cc3)cc2)cc1] 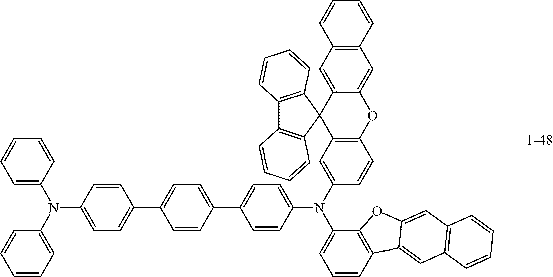 [SMILES: c1ccc(N(c2ccccc2)c2ccc(-c3ccc(-c4ccc(N(c5ccc6c(c5)C5(c7cc8ccccc8cc7O6)c6ccccc6-c6ccccc65)c5cccc6c5oc5cc7ccccc7cc56)cc4)cc3)cc2)cc1] 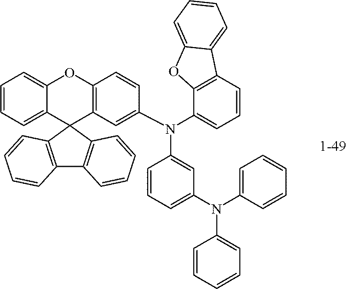 [SMILES: c1ccc(N(c2ccccc2)c2cccc(N(c3ccc4c(c3)C3(c5ccccc5O4)c4ccccc4-c4ccccc43)c3cccc4c3oc3ccccc34)c2)cc1] 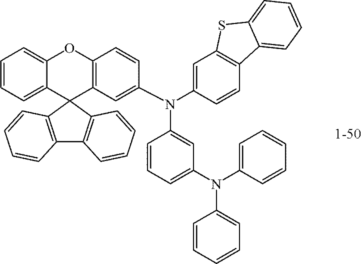 [SMILES: c1ccc(N(c2ccccc2)c2cccc(N(c3ccc4c(c3)C3(c5ccccc5O4)c4ccccc4-c4ccccc43)c3ccc4c(c3)sc3ccccc34)c2)cc1] 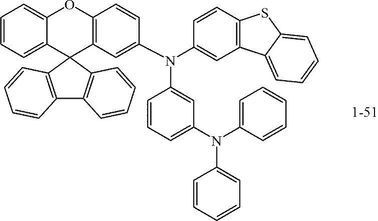 [SMILES: c1ccc(N(c2ccccc2)c2cccc(N(c3ccc4c(c3)C3(c5ccccc5O4)c4ccccc4-c4ccccc43)c3ccc4sc5ccccc5c4c3)c2)cc1] 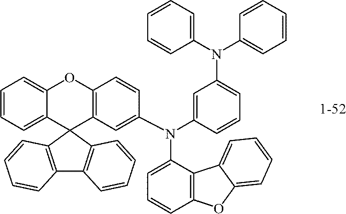 [SMILES: c1ccc(N(c2ccccc2)c2cccc(N(c3ccc4c(c3)C3(c5ccccc5O4)c4ccccc4-c4ccccc43)c3cccc4oc5ccccc5c34)c2)cc1] 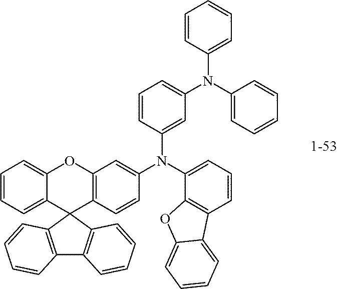 [SMILES: c1ccc(N(c2ccccc2)c2cccc(N(c3ccc4c(c3)Oc3ccccc3C43c4ccccc4-c4ccccc43)c3cccc4c3oc3ccccc34)c2)cc1] 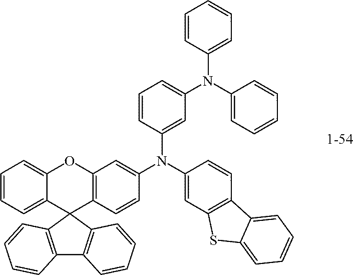 [SMILES: c1ccc(N(c2ccccc2)c2cccc(N(c3ccc4c(c3)Oc3ccccc3C43c4ccccc4-c4ccccc43)c3ccc4c(c3)sc3ccccc34)c2)cc1] 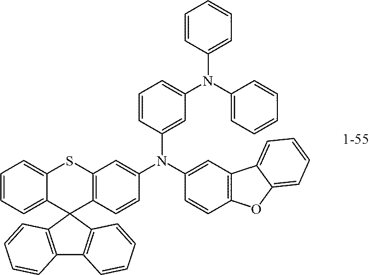 [SMILES: c1ccc(N(c2ccccc2)c2cccc(N(c3ccc4c(c3)Sc3ccccc3C43c4ccccc4-c4ccccc43)c3ccc4oc5ccccc5c4c3)c2)cc1] 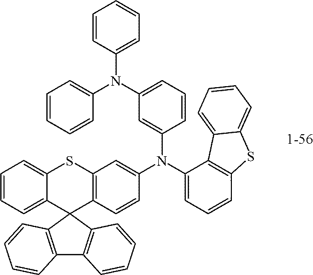 [SMILES: c1ccc(N(c2ccccc2)c2cccc(N(c3ccc4c(c3)Sc3ccccc3C43c4ccccc4-c4ccccc43)c3cccc4sc5ccccc5c34)c2)cc1] 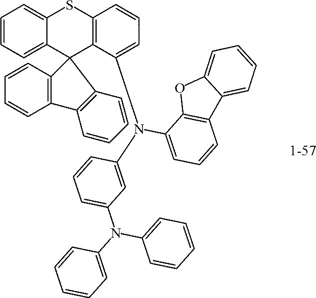 [SMILES: c1ccc(N(c2ccccc2)c2cccc(N(c3cccc4c3C3(c5ccccc5S4)c4ccccc4-c4ccccc43)c3cccc4c3oc3ccccc34)c2)cc1] 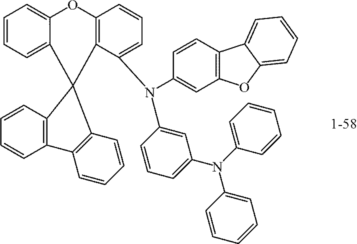 [SMILES: c1ccc(N(c2ccccc2)c2cccc(N(c3ccc4c(c3)oc3ccccc34)c3cccc4c3C3(c5ccccc5O4)c4ccccc4-c4ccccc43)c2)cc1] 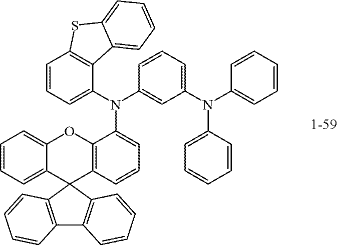 [SMILES: c1ccc(N(c2ccccc2)c2cccc(N(c3cccc4c3Oc3ccccc3C43c4ccccc4-c4ccccc43)c3cccc4sc5ccccc5c34)c2)cc1] 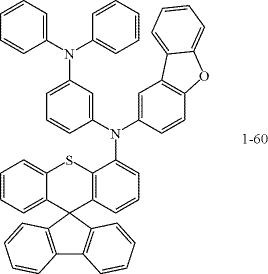 [SMILES: c1ccc(N(c2ccccc2)c2cccc(N(c3ccc4oc5ccccc5c4c3)c3cccc4c3Sc3ccccc3C43c4ccccc4-c4ccccc43)c2)cc1] 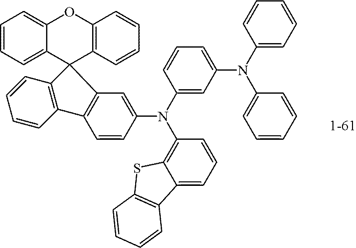 [SMILES: c1ccc(N(c2ccccc2)c2cccc(N(c3ccc4c(c3)C3(c5ccccc5Oc5ccccc53)c3ccccc3-4)c3cccc4c3sc3ccccc34)c2)cc1] 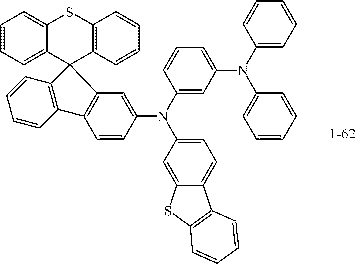 [SMILES: c1ccc(N(c2ccccc2)c2cccc(N(c3ccc4c(c3)C3(c5ccccc5Sc5ccccc53)c3ccccc3-4)c3ccc4c(c3)sc3ccccc34)c2)cc1] 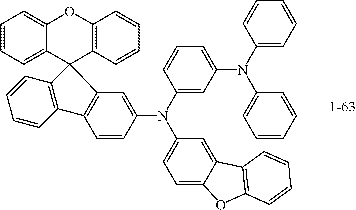 [SMILES: c1ccc(N(c2ccccc2)c2cccc(N(c3ccc4c(c3)C3(c5ccccc5Oc5ccccc53)c3ccccc3-4)c3ccc4oc5ccccc5c4c3)c2)cc1] 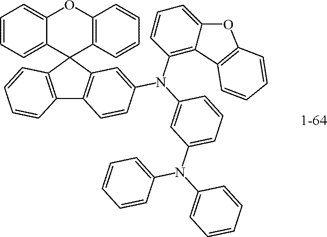 [SMILES: c1ccc(N(c2ccccc2)c2cccc(N(c3ccc4c(c3)C3(c5ccccc5Oc5ccccc53)c3ccccc3-4)c3cccc4oc5ccccc5c34)c2)cc1] 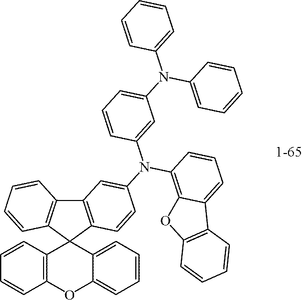 [SMILES: c1ccc(N(c2ccccc2)c2cccc(N(c3ccc4c(c3)-c3ccccc3C43c4ccccc4Oc4ccccc43)c3cccc4c3oc3ccccc34)c2)cc1] 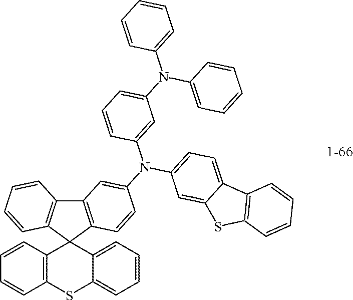 [SMILES: c1ccc(N(c2ccccc2)c2cccc(N(c3ccc4c(c3)-c3ccccc3C43c4ccccc4Sc4ccccc43)c3ccc4c(c3)sc3ccccc34)c2)cc1] 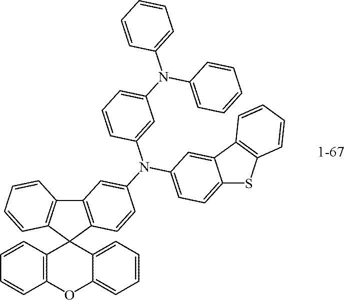 [SMILES: c1ccc(N(c2ccccc2)c2cccc(N(c3ccc4c(c3)-c3ccccc3C43c4ccccc4Oc4ccccc43)c3ccc4sc5ccccc5c4c3)c2)cc1] 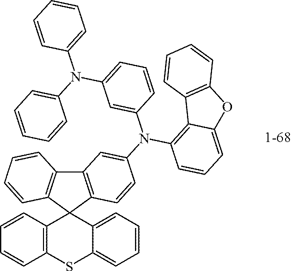 [SMILES: c1ccc(N(c2ccccc2)c2cccc(N(c3ccc4c(c3)-c3ccccc3C43c4ccccc4Sc4ccccc43)c3cccc4oc5ccccc5c34)c2)cc1] 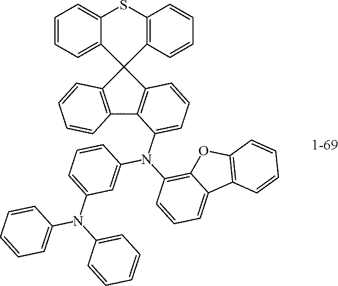 [SMILES: c1ccc(N(c2ccccc2)c2cccc(N(c3cccc4c3-c3ccccc3C43c4ccccc4Sc4ccccc43)c3cccc4c3oc3ccccc34)c2)cc1] 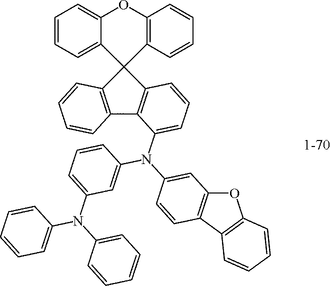 [SMILES: c1ccc(N(c2ccccc2)c2cccc(N(c3ccc4c(c3)oc3ccccc34)c3cccc4c3-c3ccccc3C43c4ccccc4Oc4ccccc43)c2)cc1] 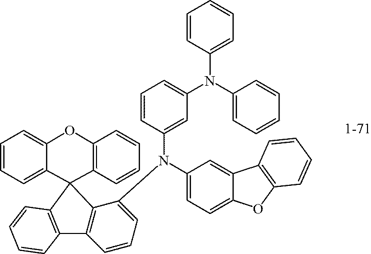 [SMILES: c1ccc(N(c2ccccc2)c2cccc(N(c3ccc4oc5ccccc5c4c3)c3cccc4c3C3(c5ccccc5Oc5ccccc53)c3ccccc3-4)c2)cc1] 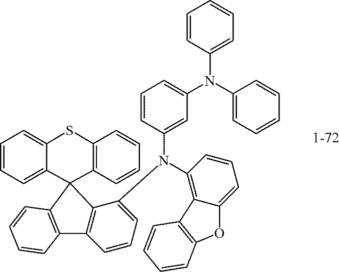 [SMILES: c1ccc(N(c2ccccc2)c2cccc(N(c3cccc4c3C3(c5ccccc5Sc5ccccc53)c3ccccc3-4)c3cccc4oc5ccccc5c34)c2)cc1] 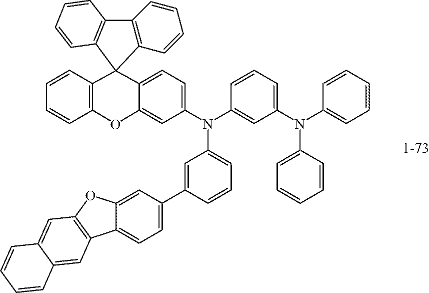 [SMILES: c1ccc(N(c2ccccc2)c2cccc(N(c3cccc(-c4ccc5c(c4)oc4cc6ccccc6cc45)c3)c3ccc4c(c3)Oc3ccccc3C43c4ccccc4-c4ccccc43)c2)cc1] 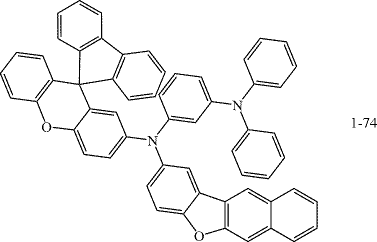 [SMILES: c1ccc(N(c2ccccc2)c2cccc(N(c3ccc4c(c3)C3(c5ccccc5O4)c4ccccc4-c4ccccc43)c3ccc4oc5cc6ccccc6cc5c4c3)c2)cc1] 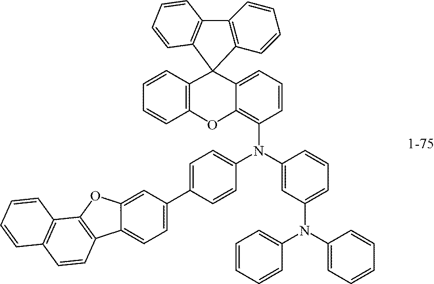 [SMILES: c1ccc(N(c2ccccc2)c2cccc(N(c3ccc(-c4ccc5c(c4)oc4c6ccccc6ccc54)cc3)c3cccc4c3Oc3ccccc3C43c4ccccc4-c4ccccc43)c2)cc1] 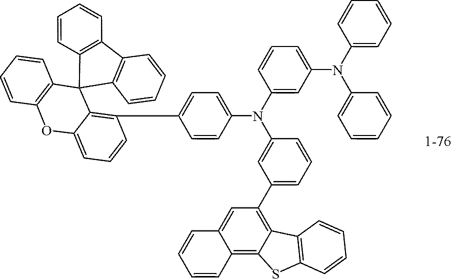 [SMILES: c1ccc(N(c2ccccc2)c2cccc(N(c3ccc(-c4cccc5c4C4(c6ccccc6O5)c5ccccc5-c5ccccc54)cc3)c3cccc(-c4cc5ccccc5c5sc6ccccc6c45)c3)c2)cc1] 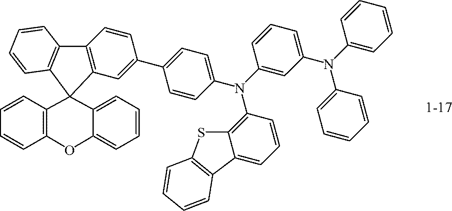 [SMILES: c1ccc(N(c2ccccc2)c2cccc(N(c3ccc(-c4ccc5c(c4)C4(c6ccccc6Oc6ccccc64)c4ccccc4-5)cc3)c3cccc4c3sc3ccccc34)c2)cc1] 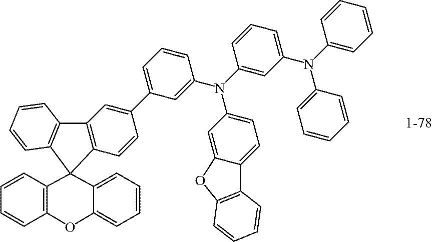 [SMILES: c1ccc(N(c2ccccc2)c2cccc(N(c3cccc(-c4ccc5c(c4)-c4ccccc4C54c5ccccc5Oc5ccccc54)c3)c3ccc4c(c3)oc3ccccc34)c2)cc1] 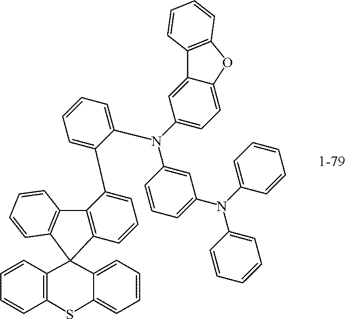 [SMILES: c1ccc(N(c2ccccc2)c2cccc(N(c3ccc4oc5ccccc5c4c3)c3ccccc3-c3cccc4c3-c3ccccc3C43c4ccccc4Sc4ccccc43)c2)cc1] 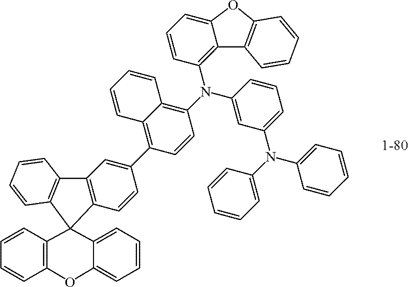 [SMILES: c1ccc(N(c2ccccc2)c2cccc(N(c3ccc(-c4ccc5c(c4)-c4ccccc4C54c5ccccc5Oc5ccccc54)c4ccccc34)c3cccc4oc5ccccc5c34)c2)cc1] 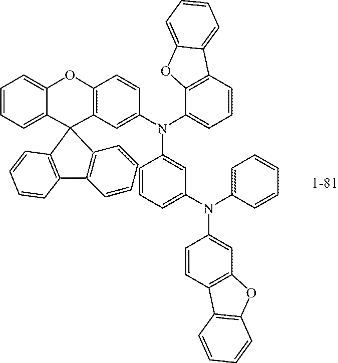 [SMILES: c1ccc(N(c2cccc(N(c3ccc4c(c3)C3(c5ccccc5O4)c4ccccc4-c4ccccc43)c3cccc4c3oc3ccccc34)c2)c2ccc3c(c2)oc2ccccc23)cc1] 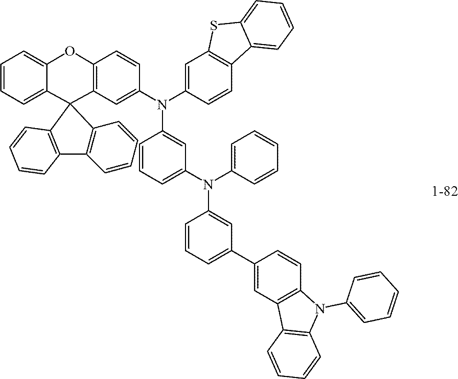 [SMILES: c1ccc(N(c2cccc(-c3ccc4c(c3)c3ccccc3n4-c3ccccc3)c2)c2cccc(N(c3ccc4c(c3)C3(c5ccccc5O4)c4ccccc4-c4ccccc43)c3ccc4c(c3)sc3ccccc34)c2)cc1] 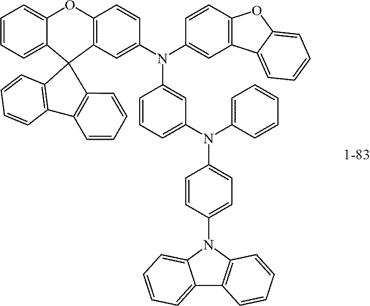 [SMILES: c1ccc(N(c2ccc(-n3c4ccccc4c4ccccc43)cc2)c2cccc(N(c3ccc4c(c3)C3(c5ccccc5O4)c4ccccc4-c4ccccc43)c3ccc4oc5ccccc5c4c3)c2)cc1] 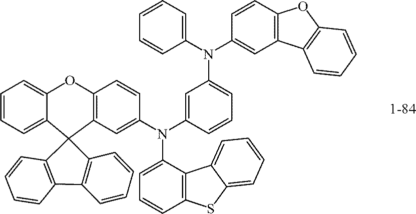 [SMILES: c1ccc(N(c2cccc(N(c3ccc4c(c3)C3(c5ccccc5O4)c4ccccc4-c4ccccc43)c3cccc4sc5ccccc5c34)c2)c2ccc3oc4ccccc4c3c2)cc1] 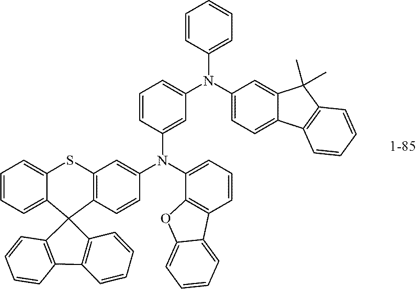 [SMILES: CC1(C)c2ccccc2-c2ccc(N(c3ccccc3)c3cccc(N(c4ccc5c(c4)Sc4ccccc4C54c5ccccc5-c5ccccc54)c4cccc5c4oc4ccccc45)c3)cc21] 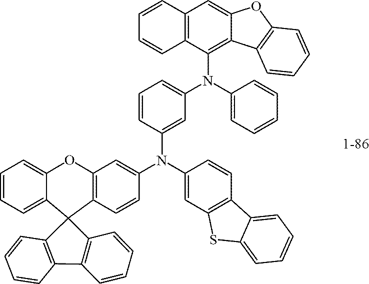 [SMILES: c1ccc(N(c2cccc(N(c3ccc4c(c3)Oc3ccccc3C43c4ccccc4-c4ccccc43)c3ccc4c(c3)sc3ccccc34)c2)c2c3ccccc3cc3oc4ccccc4c23)cc1] 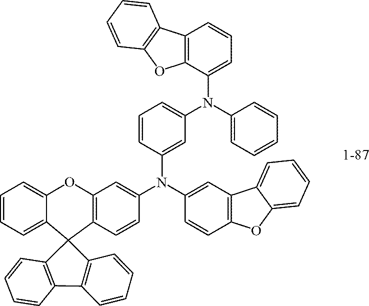 [SMILES: c1ccc(N(c2cccc(N(c3ccc4c(c3)Oc3ccccc3C43c4ccccc4-c4ccccc43)c3ccc4oc5ccccc5c4c3)c2)c2cccc3c2oc2ccccc23)cc1] 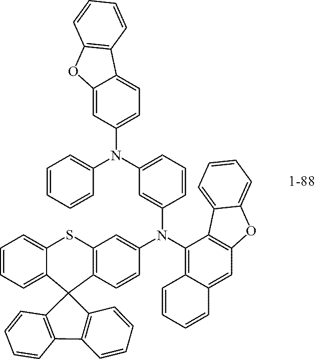 [SMILES: c1ccc(N(c2cccc(N(c3ccc4c(c3)Sc3ccccc3C43c4ccccc4-c4ccccc43)c3c4ccccc4cc4oc5ccccc5c34)c2)c2ccc3c(c2)oc2ccccc23)cc1] 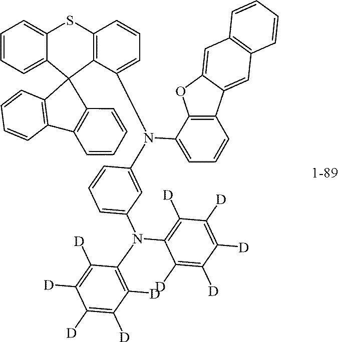 [SMILES: [2H]c1c([2H])c([2H])c(N(c2cccc(N(c3cccc4c3C3(c5ccccc5S4)c4ccccc4-c4ccccc43)c3cccc4c3oc3cc5ccccc5cc34)c2)c2c([2H])c([2H])c([2H])c([2H])c2[2H])c([2H])c1[2H]] 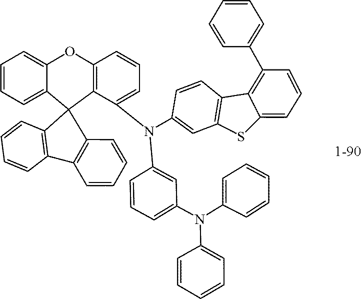 [SMILES: c1ccc(-c2cccc3sc4cc(N(c5cccc(N(c6ccccc6)c6ccccc6)c5)c5cccc6c5C5(c7ccccc7O6)c6ccccc6-c6ccccc65)ccc4c23)cc1] 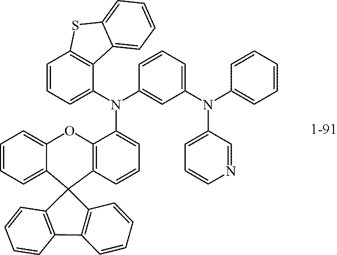 [SMILES: c1ccc(N(c2cccnc2)c2cccc(N(c3cccc4c3Oc3ccccc3C43c4ccccc4-c4ccccc43)c3cccc4sc5ccccc5c34)c2)cc1] 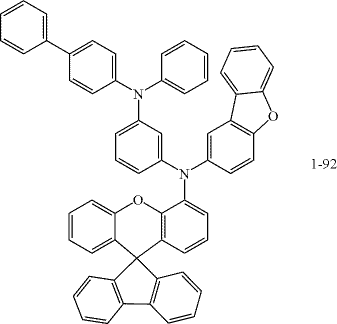 [SMILES: c1ccc(-c2ccc(N(c3ccccc3)c3cccc(N(c4ccc5oc6ccccc6c5c4)c4cccc5c4Oc4ccccc4C54c5ccccc5-c5ccccc54)c3)cc2)cc1] 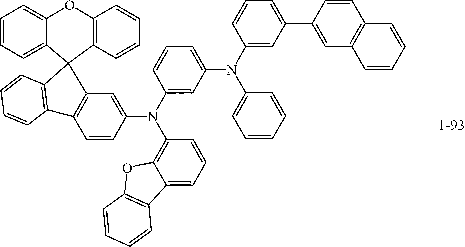 [SMILES: c1ccc(N(c2cccc(-c3ccc4ccccc4c3)c2)c2cccc(N(c3ccc4c(c3)C3(c5ccccc5Oc5ccccc53)c3ccccc3-4)c3cccc4c3oc3ccccc34)c2)cc1] 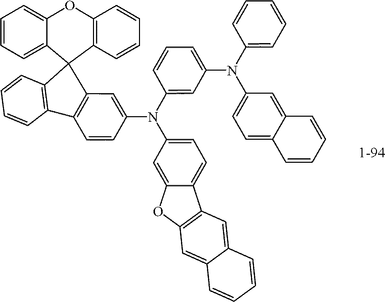 [SMILES: c1ccc(N(c2cccc(N(c3ccc4c(c3)C3(c5ccccc5Oc5ccccc53)c3ccccc3-4)c3ccc4c(c3)oc3cc5ccccc5cc34)c2)c2ccc3ccccc3c2)cc1] 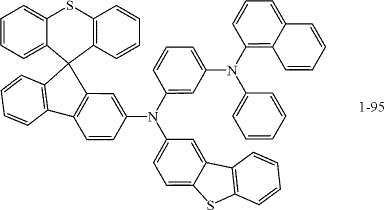 [SMILES: c1ccc(N(c2cccc(N(c3ccc4c(c3)C3(c5ccccc5Sc5ccccc53)c3ccccc3-4)c3ccc4sc5ccccc5c4c3)c2)c2cccc3ccccc23)cc1] 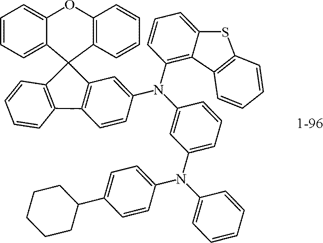 [SMILES: c1ccc(N(c2ccc(C3CCCCC3)cc2)c2cccc(N(c3ccc4c(c3)C3(c5ccccc5Oc5ccccc53)c3ccccc3-4)c3cccc4sc5ccccc5c34)c2)cc1] 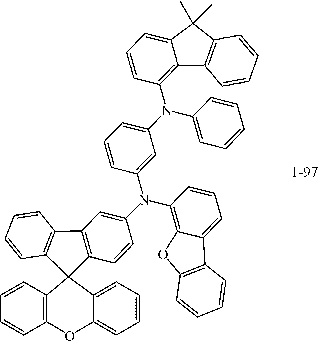 [SMILES: CC1(C)c2ccccc2-c2c(N(c3ccccc3)c3cccc(N(c4ccc5c(c4)-c4ccccc4C54c5ccccc5Oc5ccccc54)c4cccc5c4oc4ccccc45)c3)cccc21] 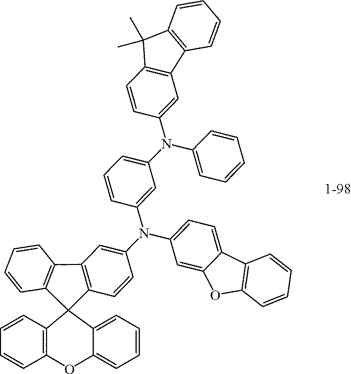 [SMILES: CC1(C)c2ccccc2-c2cc(N(c3ccccc3)c3cccc(N(c4ccc5c(c4)-c4ccccc4C54c5ccccc5Oc5ccccc54)c4ccc5c(c4)oc4ccccc45)c3)ccc21] 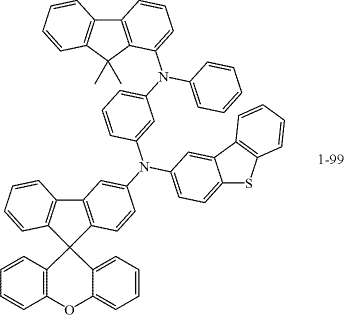 [SMILES: CC1(C)c2ccccc2-c2cccc(N(c3ccccc3)c3cccc(N(c4ccc5c(c4)-c4ccccc4C54c5ccccc5Oc5ccccc54)c4ccc5sc6ccccc6c5c4)c3)c21] 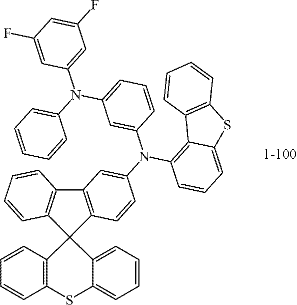 [SMILES: Fc1cc(F)cc(N(c2ccccc2)c2cccc(N(c3ccc4c(c3)-c3ccccc3C43c4ccccc4Sc4ccccc43)c3cccc4sc5ccccc5c34)c2)c1] 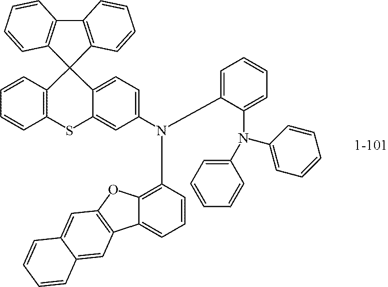 [SMILES: c1ccc(N(c2ccccc2)c2ccccc2N(c2ccc3c(c2)Sc2ccccc2C32c3ccccc3-c3ccccc32)c2cccc3c2oc2cc4ccccc4cc23)cc1] 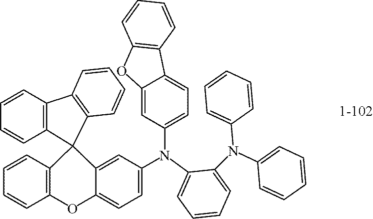 [SMILES: c1ccc(N(c2ccccc2)c2ccccc2N(c2ccc3c(c2)C2(c4ccccc4O3)c3ccccc3-c3ccccc32)c2ccc3c(c2)oc2ccccc23)cc1] 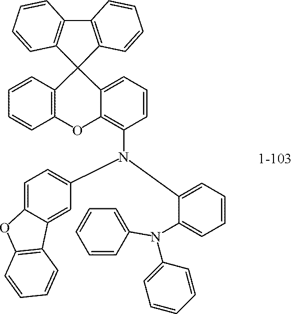 [SMILES: c1ccc(N(c2ccccc2)c2ccccc2N(c2ccc3oc4ccccc4c3c2)c2cccc3c2Oc2ccccc2C32c3ccccc3-c3ccccc32)cc1] 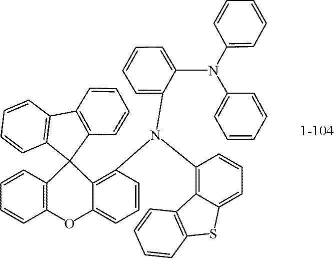 [SMILES: c1ccc(N(c2ccccc2)c2ccccc2N(c2cccc3c2C2(c4ccccc4O3)c3ccccc3-c3ccccc32)c2cccc3sc4ccccc4c23)cc1] 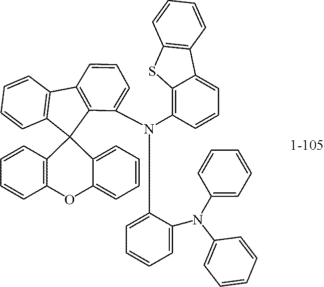 [SMILES: c1ccc(N(c2ccccc2)c2ccccc2N(c2cccc3c2C2(c4ccccc4Oc4ccccc42)c2ccccc2-3)c2cccc3c2sc2ccccc23)cc1] 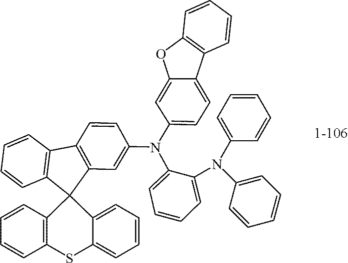 [SMILES: c1ccc(N(c2ccccc2)c2ccccc2N(c2ccc3c(c2)C2(c4ccccc4Sc4ccccc42)c2ccccc2-3)c2ccc3c(c2)oc2ccccc23)cc1] 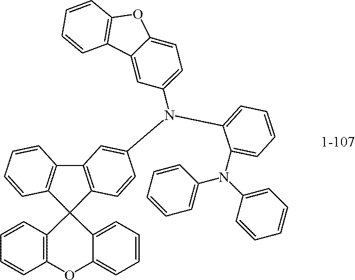 [SMILES: c1ccc(N(c2ccccc2)c2ccccc2N(c2ccc3c(c2)-c2ccccc2C32c3ccccc3Oc3ccccc32)c2ccc3oc4ccccc4c3c2)cc1] 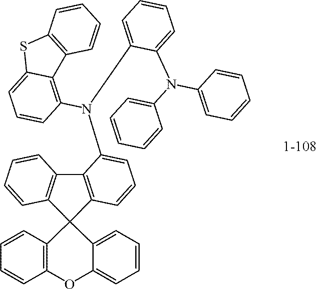 [SMILES: c1ccc(N(c2ccccc2)c2ccccc2N(c2cccc3c2-c2ccccc2C32c3ccccc3Oc3ccccc32)c2cccc3sc4ccccc4c23)cc1] 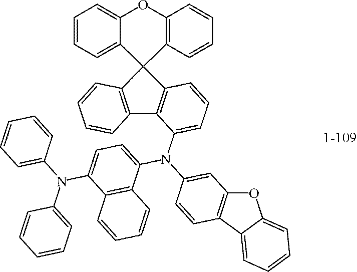 [SMILES: c1ccc(N(c2ccccc2)c2ccc(N(c3ccc4c(c3)oc3ccccc34)c3cccc4c3-c3ccccc3C43c4ccccc4Oc4ccccc43)c3ccccc23)cc1] 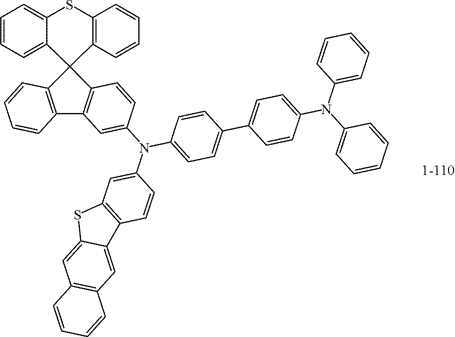 [SMILES: c1ccc(N(c2ccccc2)c2ccc(-c3ccc(N(c4ccc5c(c4)-c4ccccc4C54c5ccccc5Sc5ccccc54)c4ccc5c(c4)sc4cc6ccccc6cc45)cc3)cc2)cc1] 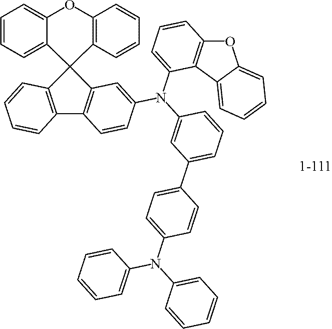 [SMILES: c1ccc(N(c2ccccc2)c2ccc(-c3cccc(N(c4ccc5c(c4)C4(c6ccccc6Oc6ccccc64)c4ccccc4-5)c4cccc5oc6ccccc6c45)c3)cc2)cc1] 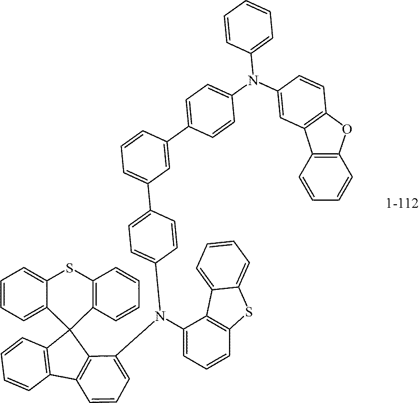 [SMILES: c1ccc(N(c2ccc(-c3cccc(-c4ccc(N(c5cccc6c5C5(c7ccccc7Sc7ccccc75)c5ccccc5-6)c5cccc6sc7ccccc7c56)cc4)c3)cc2)c2ccc3oc4ccccc4c3c2)cc1] 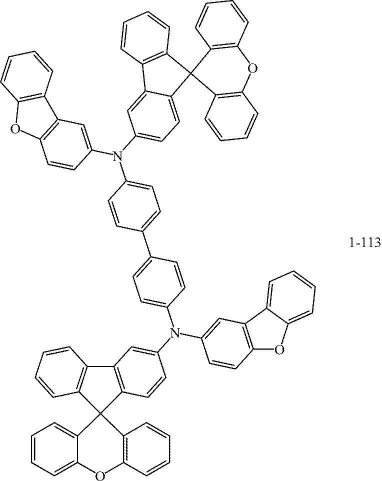 [SMILES: c1ccc2c(c1)Oc1ccccc1C21c2ccccc2-c2cc(N(c3ccc(-c4ccc(N(c5ccc6c(c5)-c5ccccc5C65c6ccccc6Oc6ccccc65)c5ccc6oc7ccccc7c6c5)cc4)cc3)c3ccc4oc5ccccc5c4c3)ccc21] 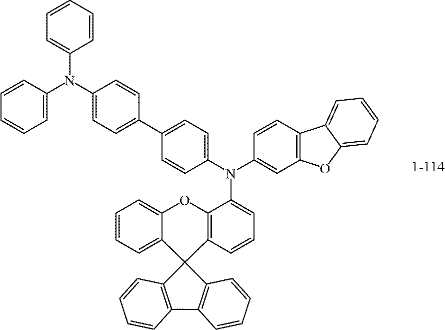 [SMILES: c1ccc(N(c2ccccc2)c2ccc(-c3ccc(N(c4ccc5c(c4)oc4ccccc45)c4cccc5c4Oc4ccccc4C54c5ccccc5-c5ccccc54)cc3)cc2)cc1] 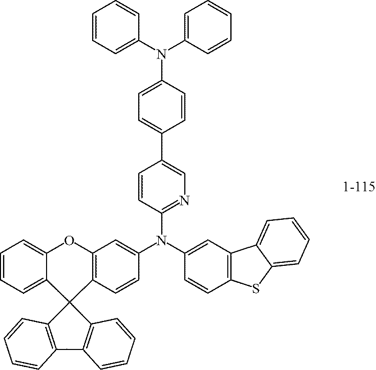 [SMILES: c1ccc(N(c2ccccc2)c2ccc(-c3ccc(N(c4ccc5c(c4)Oc4ccccc4C54c5ccccc5-c5ccccc54)c4ccc5sc6ccccc6c5c4)nc3)cc2)cc1] 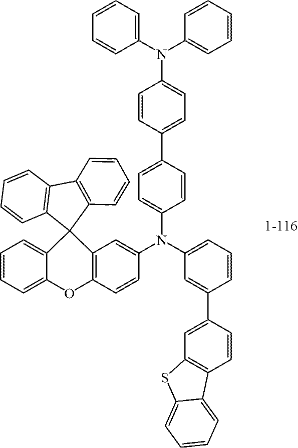 [SMILES: c1ccc(N(c2ccccc2)c2ccc(-c3ccc(N(c4cccc(-c5ccc6c(c5)sc5ccccc56)c4)c4ccc5c(c4)C4(c6ccccc6O5)c5ccccc5-c5ccccc54)cc3)cc2)cc1] 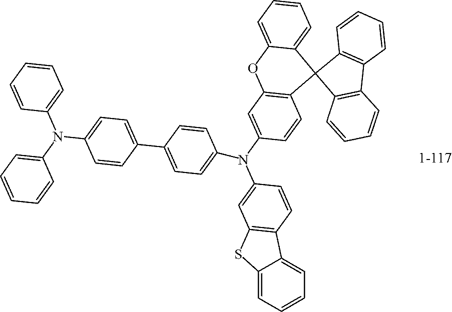 [SMILES: c1ccc(N(c2ccccc2)c2ccc(-c3ccc(N(c4ccc5c(c4)Oc4ccccc4C54c5ccccc5-c5ccccc54)c4ccc5c(c4)sc4ccccc45)cc3)cc2)cc1] 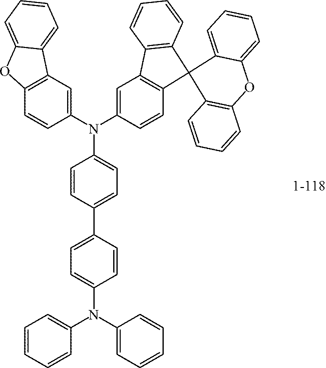 [SMILES: c1ccc(N(c2ccccc2)c2ccc(-c3ccc(N(c4ccc5c(c4)-c4ccccc4C54c5ccccc5Oc5ccccc54)c4ccc5oc6ccccc6c5c4)cc3)cc2)cc1] 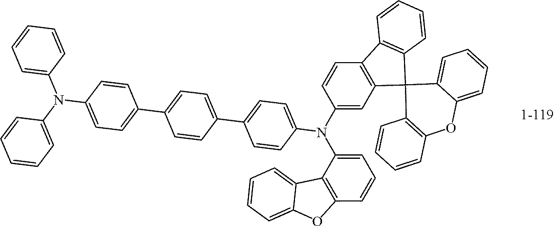 [SMILES: c1ccc(N(c2ccccc2)c2ccc(-c3ccc(-c4ccc(N(c5ccc6c(c5)C5(c7ccccc7Oc7ccccc75)c5ccccc5-6)c5cccc6oc7ccccc7c56)cc4)cc3)cc2)cc1] 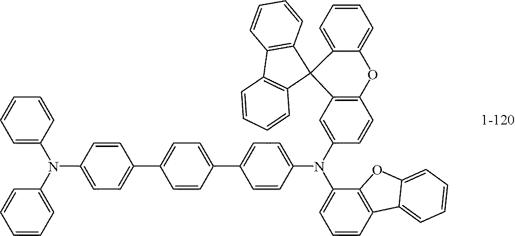 [SMILES: c1ccc(N(c2ccccc2)c2ccc(-c3ccc(-c4ccc(N(c5ccc6c(c5)C5(c7ccccc7O6)c6ccccc6-c6ccccc65)c5cccc6c5oc5ccccc56)cc4)cc3)cc2)cc1] 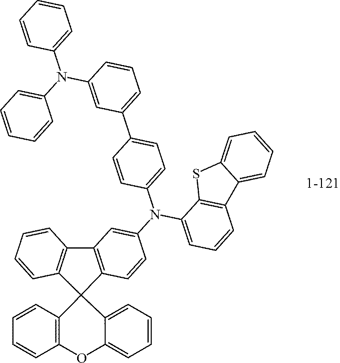 [SMILES: c1ccc(N(c2ccccc2)c2cccc(-c3ccc(N(c4ccc5c(c4)-c4ccccc4C54c5ccccc5Oc5ccccc54)c4cccc5c4sc4ccccc45)cc3)c2)cc1] 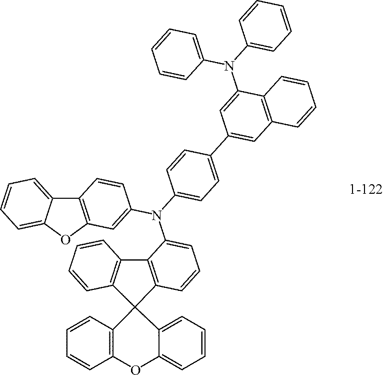 [SMILES: c1ccc(N(c2ccccc2)c2cc(-c3ccc(N(c4ccc5c(c4)oc4ccccc45)c4cccc5c4-c4ccccc4C54c5ccccc5Oc5ccccc54)cc3)cc3ccccc23)cc1] 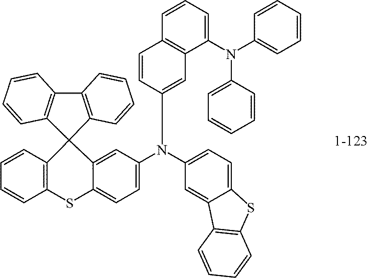 [SMILES: c1ccc(N(c2ccccc2)c2cccc3ccc(N(c4ccc5c(c4)C4(c6ccccc6S5)c5ccccc5-c5ccccc54)c4ccc5sc6ccccc6c5c4)cc23)cc1] 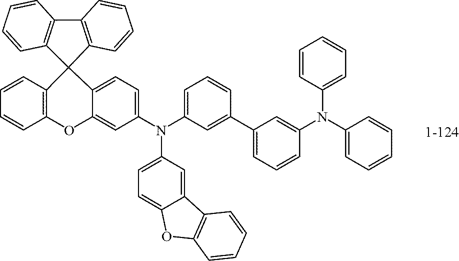 [SMILES: c1ccc(N(c2ccccc2)c2cccc(-c3cccc(N(c4ccc5c(c4)Oc4ccccc4C54c5ccccc5-c5ccccc54)c4ccc5oc6ccccc6c5c4)c3)c2)cc1] 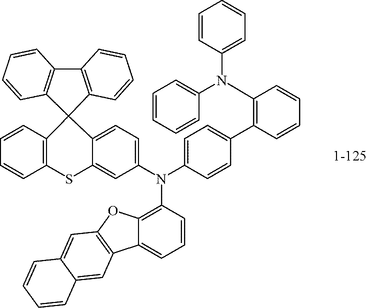 [SMILES: c1ccc(N(c2ccccc2)c2ccccc2-c2ccc(N(c3ccc4c(c3)Sc3ccccc3C43c4ccccc4-c4ccccc43)c3cccc4c3oc3cc5ccccc5cc34)cc2)cc1] 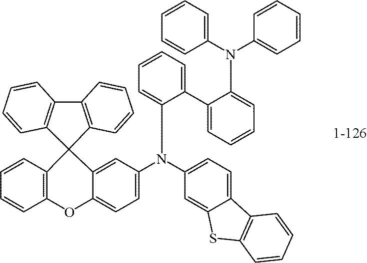 [SMILES: c1ccc(N(c2ccccc2)c2ccccc2-c2ccccc2N(c2ccc3c(c2)C2(c4ccccc4O3)c3ccccc3-c3ccccc32)c2ccc3c(c2)sc2ccccc23)cc1] 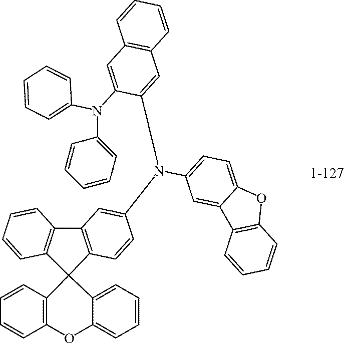 [SMILES: c1ccc(N(c2ccccc2)c2cc3ccccc3cc2N(c2ccc3c(c2)-c2ccccc2C32c3ccccc3Oc3ccccc32)c2ccc3oc4ccccc4c3c2)cc1] 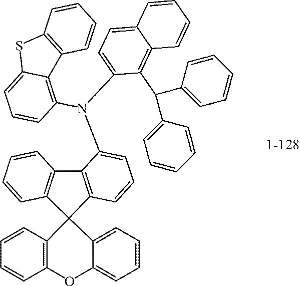 [SMILES: c1ccc(C(c2ccccc2)c2c(N(c3cccc4c3-c3ccccc3C43c4ccccc4Oc4ccccc43)c3cccc4sc5ccccc5c34)ccc3ccccc23)cc1] 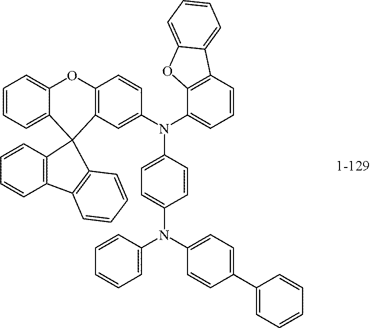 [SMILES: c1ccc(-c2ccc(N(c3ccccc3)c3ccc(N(c4ccc5c(c4)C4(c6ccccc6O5)c5ccccc5-c5ccccc54)c4cccc5c4oc4ccccc45)cc3)cc2)cc1] 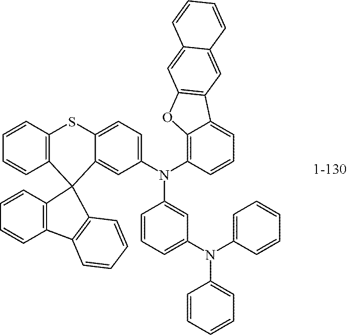 [SMILES: c1ccc(N(c2ccccc2)c2cccc(N(c3ccc4c(c3)C3(c5ccccc5S4)c4ccccc4-c4ccccc43)c3cccc4c3oc3cc5ccccc5cc34)c2)cc1] 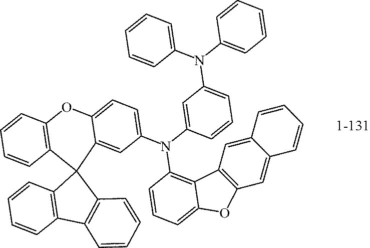 [SMILES: c1ccc(N(c2ccccc2)c2cccc(N(c3ccc4c(c3)C3(c5ccccc5O4)c4ccccc4-c4ccccc43)c3cccc4oc5cc6ccccc6cc5c34)c2)cc1] 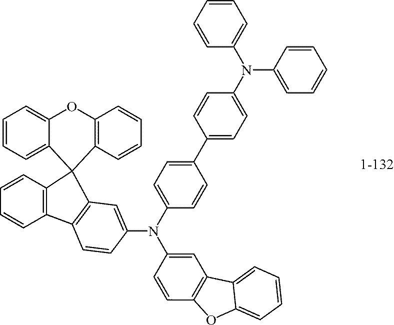 [SMILES: c1ccc(N(c2ccccc2)c2ccc(-c3ccc(N(c4ccc5c(c4)C4(c6ccccc6Oc6ccccc64)c4ccccc4-5)c4ccc5oc6ccccc6c5c4)cc3)cc2)cc1] 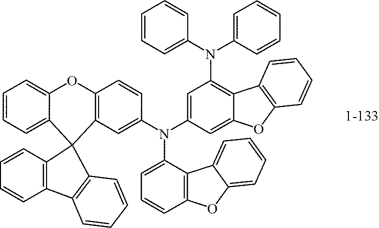 [SMILES: c1ccc(N(c2ccccc2)c2cc(N(c3ccc4c(c3)C3(c5ccccc5O4)c4ccccc4-c4ccccc43)c3cccc4oc5ccccc5c34)cc3oc4ccccc4c23)cc1] 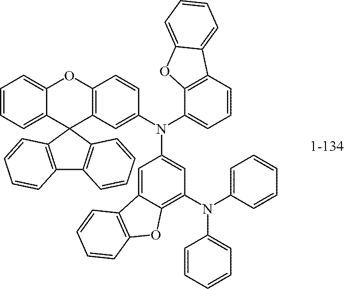 [SMILES: c1ccc(N(c2ccccc2)c2cc(N(c3ccc4c(c3)C3(c5ccccc5O4)c4ccccc4-c4ccccc43)c3cccc4c3oc3ccccc34)cc3c2oc2ccccc23)cc1] 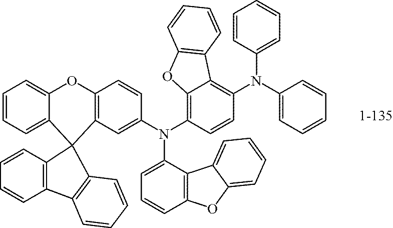 [SMILES: c1ccc(N(c2ccccc2)c2ccc(N(c3ccc4c(c3)C3(c5ccccc5O4)c4ccccc4-c4ccccc43)c3cccc4oc5ccccc5c34)c3oc4ccccc4c23)cc1] 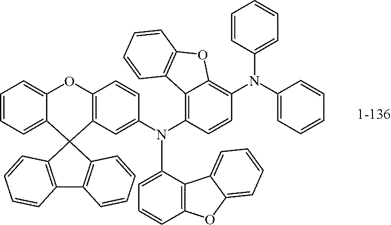 [SMILES: c1ccc(N(c2ccccc2)c2ccc(N(c3ccc4c(c3)C3(c5ccccc5O4)c4ccccc4-c4ccccc43)c3cccc4oc5ccccc5c34)c3c2oc2ccccc23)cc1] 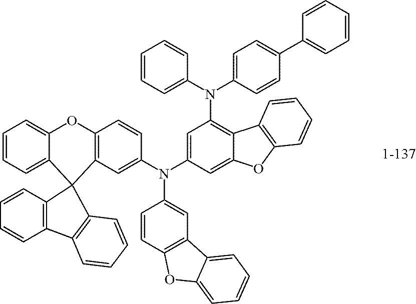 [SMILES: c1ccc(-c2ccc(N(c3ccccc3)c3cc(N(c4ccc5c(c4)C4(c6ccccc6O5)c5ccccc5-c5ccccc54)c4ccc5oc6ccccc6c5c4)cc4oc5ccccc5c34)cc2)cc1] 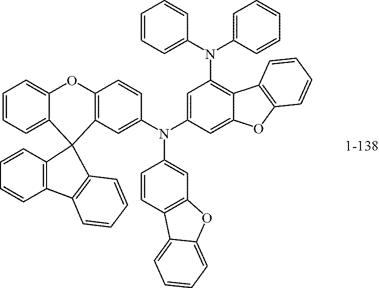 [SMILES: c1ccc(N(c2ccccc2)c2cc(N(c3ccc4c(c3)C3(c5ccccc5O4)c4ccccc4-c4ccccc43)c3ccc4c(c3)oc3ccccc34)cc3oc4ccccc4c23)cc1] 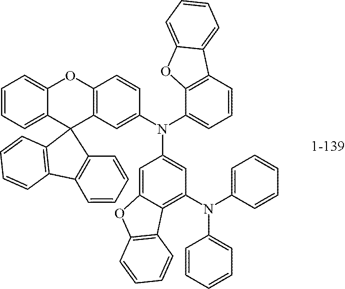 [SMILES: c1ccc(N(c2ccccc2)c2cc(N(c3ccc4c(c3)C3(c5ccccc5O4)c4ccccc4-c4ccccc43)c3cccc4c3oc3ccccc34)cc3oc4ccccc4c23)cc1] 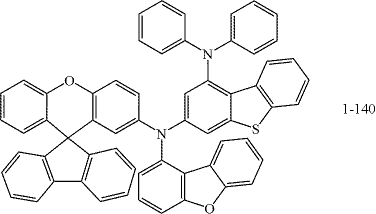 [SMILES: c1ccc(N(c2ccccc2)c2cc(N(c3ccc4c(c3)C3(c5ccccc5O4)c4ccccc4-c4ccccc43)c3cccc4oc5ccccc5c34)cc3sc4ccccc4c23)cc1] 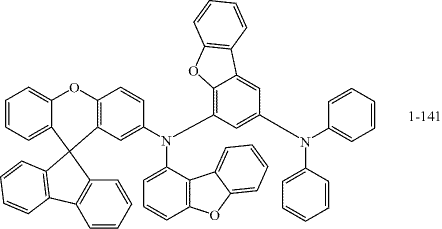 [SMILES: c1ccc(N(c2ccccc2)c2cc(N(c3ccc4c(c3)C3(c5ccccc5O4)c4ccccc4-c4ccccc43)c3cccc4oc5ccccc5c34)c3oc4ccccc4c3c2)cc1] 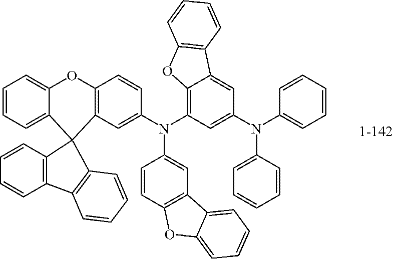 [SMILES: c1ccc(N(c2ccccc2)c2cc(N(c3ccc4c(c3)C3(c5ccccc5O4)c4ccccc4-c4ccccc43)c3ccc4oc5ccccc5c4c3)c3oc4ccccc4c3c2)cc1] 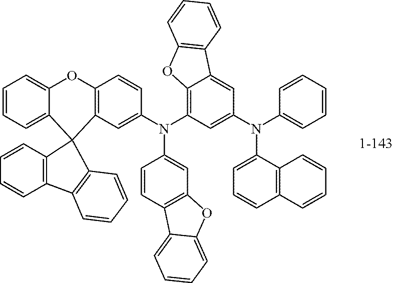 [SMILES: c1ccc(N(c2cc(N(c3ccc4c(c3)C3(c5ccccc5O4)c4ccccc4-c4ccccc43)c3ccc4c(c3)oc3ccccc34)c3oc4ccccc4c3c2)c2cccc3ccccc23)cc1] 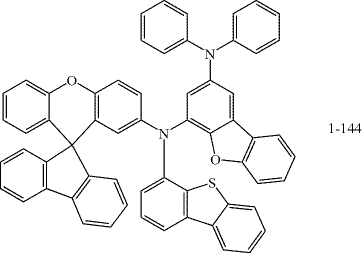 [SMILES: c1ccc(N(c2ccccc2)c2cc(N(c3ccc4c(c3)C3(c5ccccc5O4)c4ccccc4-c4ccccc43)c3cccc4c3sc3ccccc34)c3oc4ccccc4c3c2)cc1] 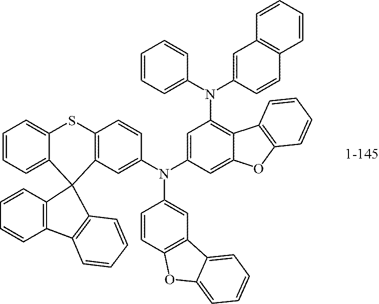 [SMILES: c1ccc(N(c2ccc3ccccc3c2)c2cc(N(c3ccc4c(c3)C3(c5ccccc5S4)c4ccccc4-c4ccccc43)c3ccc4oc5ccccc5c4c3)cc3oc4ccccc4c23)cc1] 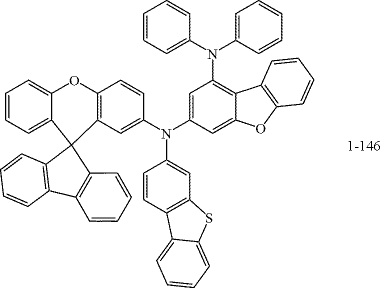 [SMILES: c1ccc(N(c2ccccc2)c2cc(N(c3ccc4c(c3)C3(c5ccccc5O4)c4ccccc4-c4ccccc43)c3ccc4c(c3)sc3ccccc34)cc3oc4ccccc4c23)cc1] 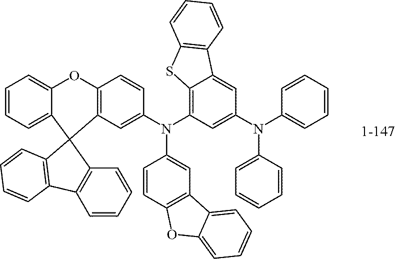 [SMILES: c1ccc(N(c2ccccc2)c2cc(N(c3ccc4c(c3)C3(c5ccccc5O4)c4ccccc4-c4ccccc43)c3ccc4oc5ccccc5c4c3)c3sc4ccccc4c3c2)cc1] 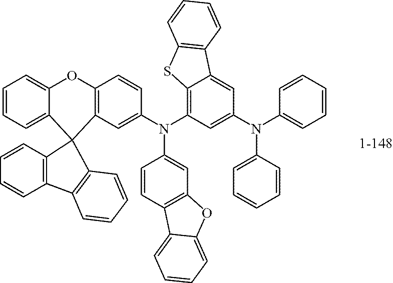 [SMILES: c1ccc(N(c2ccccc2)c2cc(N(c3ccc4c(c3)C3(c5ccccc5O4)c4ccccc4-c4ccccc43)c3ccc4c(c3)oc3ccccc34)c3sc4ccccc4c3c2)cc1] 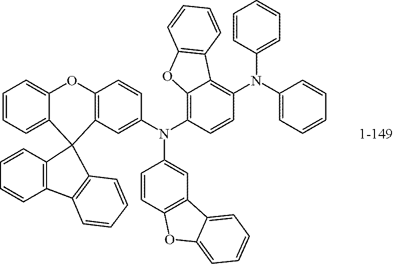 [SMILES: c1ccc(N(c2ccccc2)c2ccc(N(c3ccc4c(c3)C3(c5ccccc5O4)c4ccccc4-c4ccccc43)c3ccc4oc5ccccc5c4c3)c3oc4ccccc4c23)cc1] 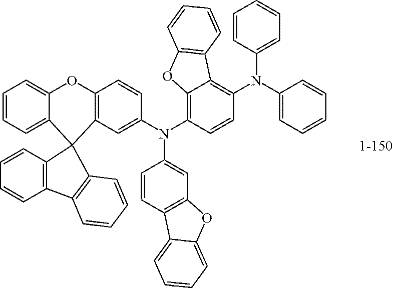 [SMILES: c1ccc(N(c2ccccc2)c2ccc(N(c3ccc4c(c3)C3(c5ccccc5O4)c4ccccc4-c4ccccc43)c3ccc4c(c3)oc3ccccc34)c3oc4ccccc4c23)cc1] 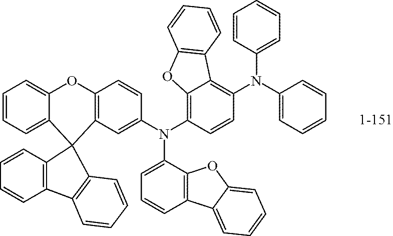 [SMILES: c1ccc(N(c2ccccc2)c2ccc(N(c3ccc4c(c3)C3(c5ccccc5O4)c4ccccc4-c4ccccc43)c3cccc4c3oc3ccccc34)c3oc4ccccc4c23)cc1] 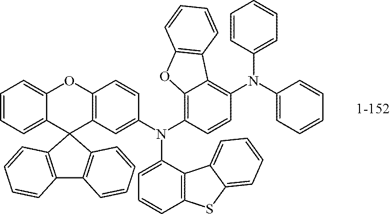 [SMILES: c1ccc(N(c2ccccc2)c2ccc(N(c3ccc4c(c3)C3(c5ccccc5O4)c4ccccc4-c4ccccc43)c3cccc4sc5ccccc5c34)c3oc4ccccc4c23)cc1] 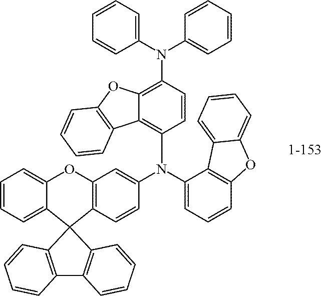 [SMILES: c1ccc(N(c2ccccc2)c2ccc(N(c3ccc4c(c3)Oc3ccccc3C43c4ccccc4-c4ccccc43)c3cccc4oc5ccccc5c34)c3c2oc2ccccc23)cc1] 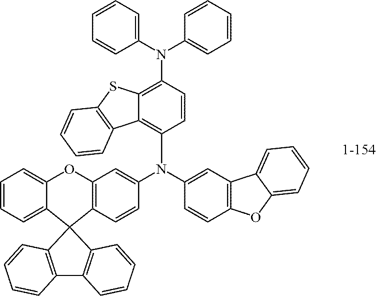 [SMILES: c1ccc(N(c2ccccc2)c2ccc(N(c3ccc4c(c3)Oc3ccccc3C43c4ccccc4-c4ccccc43)c3ccc4oc5ccccc5c4c3)c3c2sc2ccccc23)cc1] 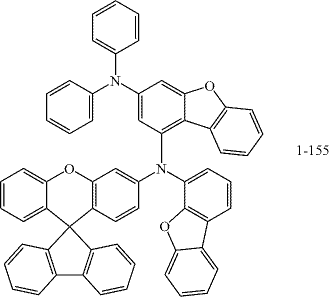 [SMILES: c1ccc(N(c2ccccc2)c2cc(N(c3ccc4c(c3)Oc3ccccc3C43c4ccccc4-c4ccccc43)c3cccc4c3oc3ccccc34)c3c(c2)oc2ccccc23)cc1] 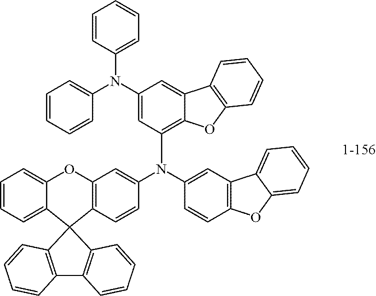 [SMILES: c1ccc(N(c2ccccc2)c2cc(N(c3ccc4c(c3)Oc3ccccc3C43c4ccccc4-c4ccccc43)c3ccc4oc5ccccc5c4c3)c3oc4ccccc4c3c2)cc1] 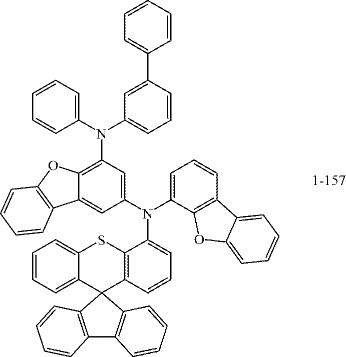 [SMILES: c1ccc(-c2cccc(N(c3ccccc3)c3cc(N(c4cccc5c4Sc4ccccc4C54c5ccccc5-c5ccccc54)c4cccc5c4oc4ccccc45)cc4c3oc3ccccc34)c2)cc1] 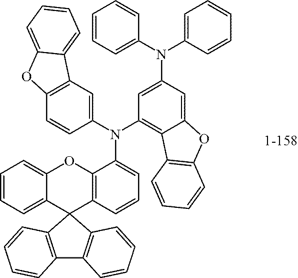 [SMILES: c1ccc(N(c2ccccc2)c2cc(N(c3ccc4oc5ccccc5c4c3)c3cccc4c3Oc3ccccc3C43c4ccccc4-c4ccccc43)c3c(c2)oc2ccccc23)cc1] 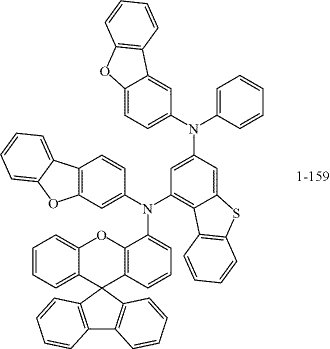 [SMILES: c1ccc(N(c2cc(N(c3ccc4c(c3)oc3ccccc34)c3cccc4c3Oc3ccccc3C43c4ccccc4-c4ccccc43)c3c(c2)sc2ccccc23)c2ccc3oc4ccccc4c3c2)cc1] 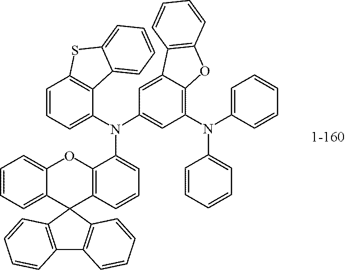 [SMILES: c1ccc(N(c2ccccc2)c2cc(N(c3cccc4c3Oc3ccccc3C43c4ccccc4-c4ccccc43)c3cccc4sc5ccccc5c34)cc3c2oc2ccccc23)cc1] 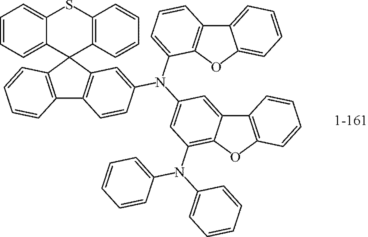 [SMILES: c1ccc(N(c2ccccc2)c2cc(N(c3ccc4c(c3)C3(c5ccccc5Sc5ccccc53)c3ccccc3-4)c3cccc4c3oc3ccccc34)cc3c2oc2ccccc23)cc1] 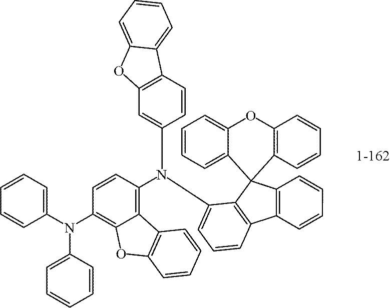 [SMILES: c1ccc(N(c2ccccc2)c2ccc(N(c3ccc4c(c3)oc3ccccc34)c3cccc4c3C3(c5ccccc5Oc5ccccc53)c3ccccc3-4)c3c2oc2ccccc23)cc1] 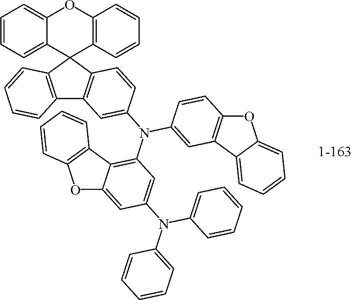 [SMILES: c1ccc(N(c2ccccc2)c2cc(N(c3ccc4c(c3)-c3ccccc3C43c4ccccc4Oc4ccccc43)c3ccc4oc5ccccc5c4c3)c3c(c2)oc2ccccc23)cc1] 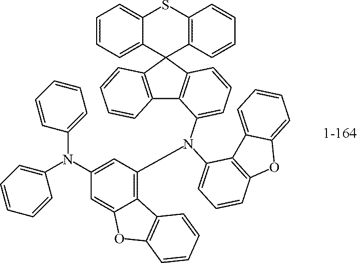 [SMILES: c1ccc(N(c2ccccc2)c2cc(N(c3cccc4c3-c3ccccc3C43c4ccccc4Sc4ccccc43)c3cccc4oc5ccccc5c34)c3c(c2)oc2ccccc23)cc1] 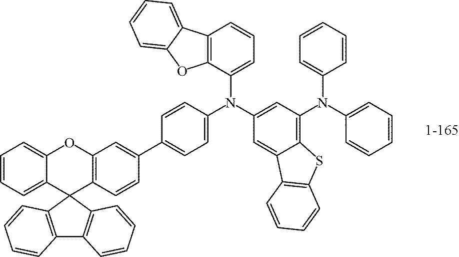 [SMILES: c1ccc(N(c2ccccc2)c2cc(N(c3ccc(-c4ccc5c(c4)Oc4ccccc4C54c5ccccc5-c5ccccc54)cc3)c3cccc4c3oc3ccccc34)cc3c2sc2ccccc23)cc1] 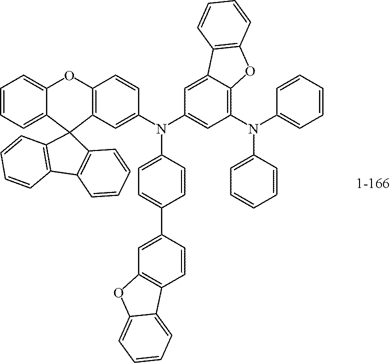 [SMILES: c1ccc(N(c2ccccc2)c2cc(N(c3ccc(-c4ccc5c(c4)oc4ccccc45)cc3)c3ccc4c(c3)C3(c5ccccc5O4)c4ccccc4-c4ccccc43)cc3c2oc2ccccc23)cc1] 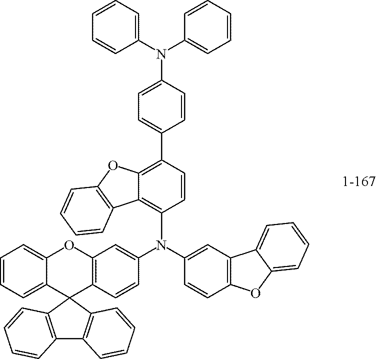 [SMILES: c1ccc(N(c2ccccc2)c2ccc(-c3ccc(N(c4ccc5c(c4)Oc4ccccc4C54c5ccccc5-c5ccccc54)c4ccc5oc6ccccc6c5c4)c4c3oc3ccccc34)cc2)cc1] 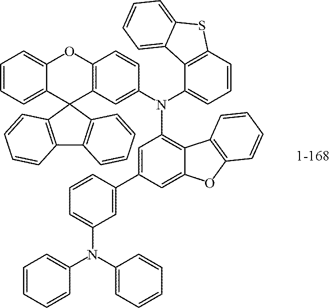 [SMILES: c1ccc(N(c2ccccc2)c2cccc(-c3cc(N(c4ccc5c(c4)C4(c6ccccc6O5)c5ccccc5-c5ccccc54)c4cccc5sc6ccccc6c45)c4c(c3)oc3ccccc34)c2)cc1] 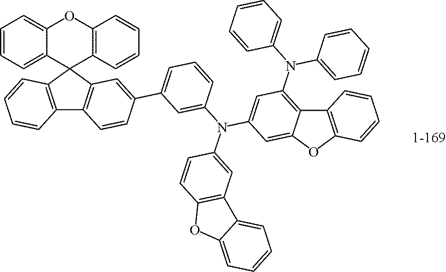 [SMILES: c1ccc(N(c2ccccc2)c2cc(N(c3cccc(-c4ccc5c(c4)C4(c6ccccc6Oc6ccccc64)c4ccccc4-5)c3)c3ccc4oc5ccccc5c4c3)cc3oc4ccccc4c23)cc1] 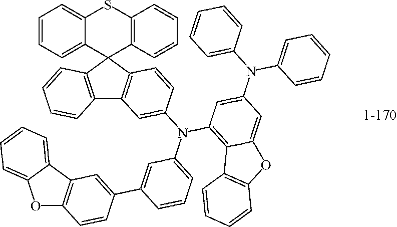 [SMILES: c1ccc(N(c2ccccc2)c2cc(N(c3cccc(-c4ccc5oc6ccccc6c5c4)c3)c3ccc4c(c3)-c3ccccc3C43c4ccccc4Sc4ccccc43)c3c(c2)oc2ccccc23)cc1] 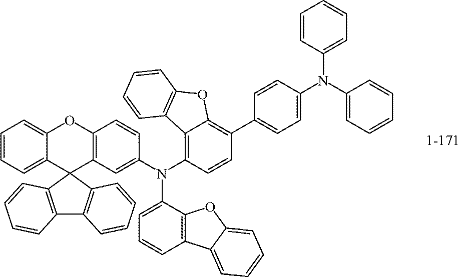 [SMILES: c1ccc(N(c2ccccc2)c2ccc(-c3ccc(N(c4ccc5c(c4)C4(c6ccccc6O5)c5ccccc5-c5ccccc54)c4cccc5c4oc4ccccc45)c4c3oc3ccccc34)cc2)cc1] 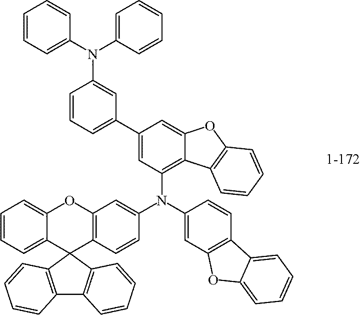 [SMILES: c1ccc(N(c2ccccc2)c2cccc(-c3cc(N(c4ccc5c(c4)Oc4ccccc4C54c5ccccc5-c5ccccc54)c4ccc5c(c4)oc4ccccc45)c4c(c3)oc3ccccc34)c2)cc1] 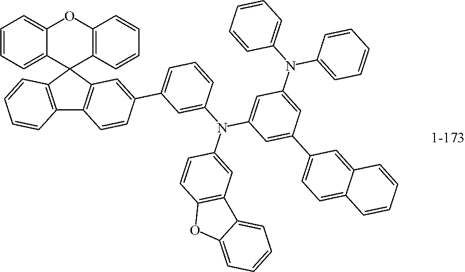 [SMILES: c1ccc(N(c2ccccc2)c2cc(-c3ccc4ccccc4c3)cc(N(c3cccc(-c4ccc5c(c4)C4(c6ccccc6Oc6ccccc64)c4ccccc4-5)c3)c3ccc4oc5ccccc5c4c3)c2)cc1] 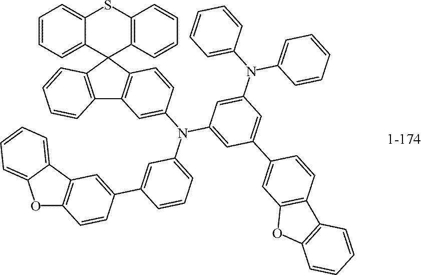 [SMILES: c1ccc(N(c2ccccc2)c2cc(-c3ccc4c(c3)oc3ccccc34)cc(N(c3cccc(-c4ccc5oc6ccccc6c5c4)c3)c3ccc4c(c3)-c3ccccc3C43c4ccccc4Sc4ccccc43)c2)cc1] 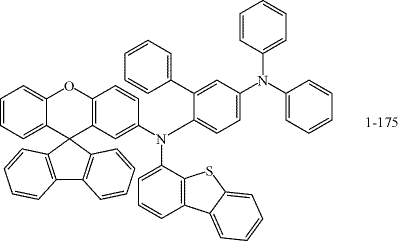 [SMILES: c1ccc(-c2cc(N(c3ccccc3)c3ccccc3)ccc2N(c2ccc3c(c2)C2(c4ccccc4O3)c3ccccc3-c3ccccc32)c2cccc3c2sc2ccccc23)cc1] 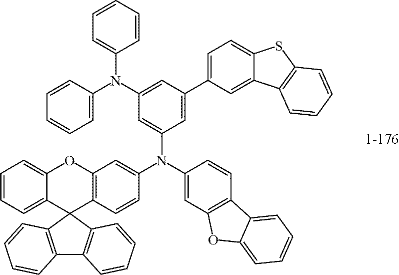 [SMILES: c1ccc(N(c2ccccc2)c2cc(-c3ccc4sc5ccccc5c4c3)cc(N(c3ccc4c(c3)Oc3ccccc3C43c4ccccc4-c4ccccc43)c3ccc4c(c3)oc3ccccc34)c2)cc1] 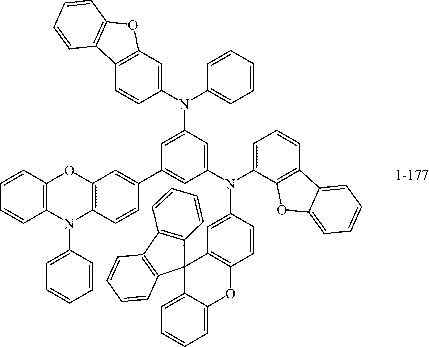 [SMILES: c1ccc(N(c2cc(-c3ccc4c(c3)Oc3ccccc3N4c3ccccc3)cc(N(c3ccc4c(c3)C3(c5ccccc5O4)c4ccccc4-c4ccccc43)c3cccc4c3oc3ccccc34)c2)c2ccc3c(c2)oc2ccccc23)cc1] 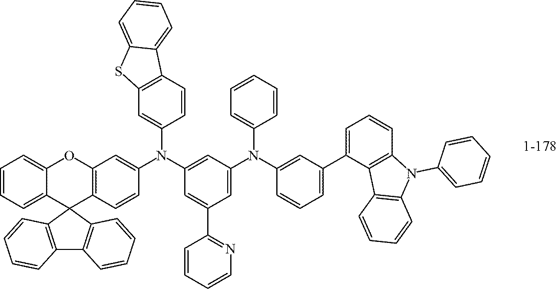 [SMILES: c1ccc(N(c2cccc(-c3cccc4c3c3ccccc3n4-c3ccccc3)c2)c2cc(-c3ccccn3)cc(N(c3ccc4c(c3)Oc3ccccc3C43c4ccccc4-c4ccccc43)c3ccc4c(c3)sc3ccccc34)c2)cc1] 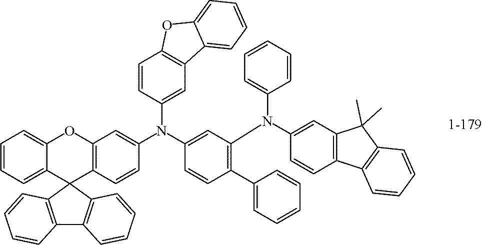 [SMILES: CC1(C)c2ccccc2-c2ccc(N(c3ccccc3)c3cc(N(c4ccc5c(c4)Oc4ccccc4C54c5ccccc5-c5ccccc54)c4ccc5oc6ccccc6c5c4)ccc3-c3ccccc3)cc21] 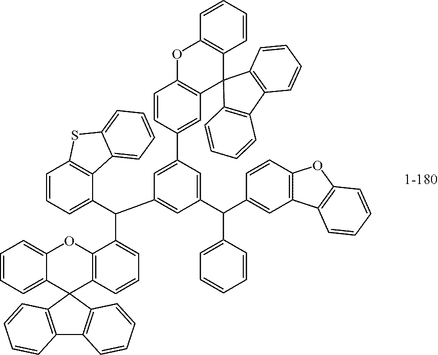 [SMILES: c1ccc(C(c2cc(-c3ccc4c(c3)C3(c5ccccc5O4)c4ccccc4-c4ccccc43)cc(C(c3cccc4c3Oc3ccccc3C43c4ccccc4-c4ccccc43)c3cccc4sc5ccccc5c34)c2)c2ccc3oc4ccccc4c3c2)cc1]
.

6. An organic electric element comprising a first electrode, a second electrode, and an organic material layer between the first electrode and the second electrode, wherein the organic material layer comprises the compound represented by Formula 1 of claim 1.

7. The organic electric element of claim 6, further comprising a layer for improving luminous efficiency, wherein the layer for improving luminous efficiency is formed on one side of the first electrode and the second electrode, and the one side is a side not facing the organic material layer.

8. The organic electric element of claim 6, wherein the compound represented by Formula 1 is comprised in a light-emitting layer of the organic material layer.

9. The organic electric element of claim 7, wherein the layer for improving luminous efficiency comprises the compound represented by Formula 1.

10. The organic electric element of claim 6, wherein the organic material layer comprises two or more stacks, and the two or more stacks each comprise a hole transport layer, a light-emitting layer and an electron transport layer formed sequentially on the first electrode.

11. The blue organic electric element of claim 10, wherein the organic material layer further comprises a charge generation layer formed between the two or more stacks.

12. An electronic device comprising a display device and a control unit for driving the display device, wherein the display device comprises the organic electric element of claim 6.

13. The electronic device of claim 12, wherein the organic electric element is selected from the group consisting of an organic electroluminescent element, an organic solar cell, an organic photo conductor, an organic transistor, an element for monochromatic illumination and a quantum dot display.
